(19) United States
(12) Patent Application Publication
(10) Pub. No.: US 2025/0128262 A1
(43) Pub. Date: Apr. 24, 2025

(54) MOLECULAR DIAGNOSTIC DEVICES AND METHODS FOR RETAINING AND MIXING REAGENTS

(71) Applicant: VISBY MEDICAL, INC., San Jose, CA (US)

(72) Inventors: Boris ANDREYEV, Foster City, CA (US); Victor BRIONES, Gilroy, CA (US); Brian CIOPYK, Los Gatos, CA (US); Paul DENTINGER, Sunol, CA (US); David DIEPERSLOOT, Scotts Valley, CA (US); Gregory C. LONEY, Los Altos, CA (US); Brandon MA, Santa Clara, CA (US); Nicholas S. MERCER, San Francisco, CA (US); Paurav PATEL, Los Altos Hills, CA (US); Ashley KWOK, Santa Clara, CA (US); Kamal KAJOUKE, San Jose, CA (US)

(73) Assignee: VISBY MEDICAL, INC., San Jose, CA (US)

(21) Appl. No.: 18/682,645

(22) PCT Filed: Aug. 11, 2022
(86) PCT No.: PCT/US2022/040101

§ 371 Date: Feb. 9, 2024

Related U.S. Application Data
(60) Provisional application No. 63/232,865, filed on Aug. 13, 2021.

(51) Int. Cl. B01L 7/00

(52) U.S. Cl. CPC B01L 7/525; B01L 2200/028; B01L 2200/0684; B01L 2200/10; B01L 2200/16; B01L 2300/0867; B01L 2300/0883; B01L 2300/1805; B01L 2400/0478; B01L 2400/0487; B01L 2400/0644

(57) ABSTRACT

A molecular diagnostic test device can include a housing and a sample preparation module within the housing. The sample preparation module defines an input reservoir and a sample input opening through which a biological sample can be loaded into the input reservoir. The sample preparation module includes a solid reagent contained within the input reservoir. The molecular diagnostic test device can include a fluid source within the housing. The fluid source is in fluid communication with the input reservoir such that upon activation of the molecular diagnostic test device, the fluid from the fluid source is injected into the input reservoir to mix the biological sample and the solid reagent within the input reservoir to form an input solution containing a target molecule.

### CROSS-REFERENCE TO RELATED APPLICATIONS

This application claims benefit of priority to U.S. Provisional Application Ser. No. 63/232,865, entitled “Molecular Diagnostic Devices and Methods for Retaining and Mixing Reagents,” filed Aug. 13, 2021, which is incorporated herein by reference in its entirety.

### BACKGROUND

The embodiments described herein relate to devices and methods for molecular diagnostic testing. More particularly, the embodiments described herein relate to disposable, self-contained devices and methods for molecular diagnostic testing.

There are over one billion infections in the U.S. each year, many of which are treated incorrectly due to inaccurate or delayed diagnostic results. Many known point of care (POC) tests have poor sensitivity (30-70%), while the more highly sensitive tests, such as those involving the specific detection of nucleic acids or molecular testing associated with a pathogenic target, are only available in laboratories. Thus, molecular diagnostics testing is often practiced in centralized laboratories. Known devices and methods for conducting laboratory-based molecular diagnostics testing, however, require trained personnel, regulated infrastructure, and expensive, high throughput instrumentation. Known high throughput laboratory equipment generally processes many (96 to 384 and more) samples at a time, therefore central lab testing is often done in batches. Known methods for processing test samples typically include processing all samples collected during a time period (e.g., a day) in one large run, resulting in a turn-around time of many hours to days after the sample is collected. Moreover, such known instrumentation and methods are designed to perform certain operations under the guidance of a skilled technician who adds reagents, oversees processing, and moves sample from step to step. Thus, although known laboratory tests and methods are very accurate, they often take considerable time, and are very expensive.

Although some known laboratory-based molecular diagnostics test methods and equipment offer flexibility (e.g., the ability to test for multiple different indications), such methods and equipment are not easily adaptable for point of care (“POC”) use or in-home use by an untrained user. Specifically, such known devices and methods are complicated to use and include expensive and sophisticated components. Thus, the use of such known laboratory-based methods and devices in a decentralized setting (e.g., POC or in-home use) would likely result in an increase in misuse, leading to inaccurate results or safety concerns. For example, many known laboratory-based systems include sophisticated optics and laser light sources, which can present a safety hazard to an untrained user. Some known systems can also require the user to handle or be exposed to reagents, which can be a safety risk for an untrained user. For example, some known systems use relatively large amounts of reagents and/or require replenishment of the reagents (e.g., within an instrument). In addition to being unsuitable for decentralized use, these known systems are also not suitable for long-term storage and shipping. Long-term storage can be desirable, for example to allow for stockpiling of assays for military applications, as a part of the CDC strategic national stockpile program, or other emergency preparedness initiatives.

Moreover, because of the flexibility offered by many known laboratory-based systems, such systems do not include lock-outs or mechanisms that prevent an untrained user from completing certain actions out of the proper sequence. For example, many known systems and methods include several distinct sample preparation operations, such as filtering, washing, lysing, and addition of sample preparation reagents to preserve the target nucleic acids. If such operations are not performed in a predetermined order and/or within predetermined time limits, the accuracy of the test can be compromised. Some known systems attempt to limit the complexities associated with sample preparation by limiting the analysis to only “clean” samples. As a result, such systems do not enable true end-to-end molecular diagnostic methods, because the detailed sample preparation must still be performed by an upstream process.

There are other potential concerns with performing decentralized testing (e.g., testing at a point-of-care, a patient's home, work sites, or other public venues) that may lead to reduced performance (e.g., reduced specificity and sensitivity, greater incidents of aborted tests, etc.). For example, test components that are shipped and stored in standard channels of commerce and/or that are used outside of a laboratory setting may be subjected to extreme ambient conditions (temperatures, pressures), vibration, change in orientation or the like. As such, pre-loaded reagents may be at a greater risk of being compromised (e.g., leaking or falling out of their container). Additionally, ensuring that all reagents and solutions are properly mixed during the administration of the test can be problematic outside of a laboratory environment. Furthermore, decentralized testing in small handheld devices (e.g., testing at a point-of-care, a patient's home, work sites, or other public venues) may lead to reduced performance due to improper mixing of biological samples reagents.

In addition to requiring sufficient accuracy, cost and availability is another important factor in implementing decentralized testing. For example, there has been increased interest in combining or “pooling” samples to allow multiple users (or specimens) to be tested with one diagnostic test. This approach increases the number of individuals that can be tested using the same amount of resources. Additionally, it is desirable to establish high volume manufacturing for test components (e.g., to increase availability and decrease cost). Such components or devices, however, can exhibit part-to-part variability (e.g., due to normal manufacturing tolerances), which can impact the overall performance. For example, variability in positioning of a detection system relative to a sample can result in undesirable variation in results.

Multiplex tests (i.e., test devices or systems that can detect multiple different target nucleic acids) are another important tool in providing a more comprehensive diagnosis without requiring multiple different patient visits, sample collection events, or test components. For example, some known multiplex tests can screen for multiple different indications (e.g., COVID-19 and multiple variants of influenza). Multiplex tests, however, generally require a higher volume of sample to test for multiple targets. Such known multiplex tests can also require greater amount of reagents (buffer solutions, PCR enzymes, or the like), which can increase costs.

Thus, a need exists for improved devices and methods for molecular diagnostic testing. In particular, a need exists for improved devices and methods that improve the viewability of the signal. A need also exists for improved devices and methods that better retain reagents. A need also exists for improved devices and methods that can employ less amounts of reagents and also improve the mixing of reagents and biological samples.

### SUMMARY

Molecular diagnostic test devices for producing an indicator of a target molecule (e.g., DNA or RNA) in the sample are described herein. In accordance with some embodiments, the molecular diagnostic test device includes a housing having an outer wall defining a viewing area. The molecular diagnostic test device also includes a sample preparation module within the housing. The sample preparation module defines an input reservoir configured to receive a biological sample. The sample preparation module is configured to produce an input solution containing a target molecule. The molecular diagnostic test device can also include a reaction module disposed within the housing, the reaction module being configured to receive the input solution containing the target molecule and amplify the target molecule to produce an output containing a target amplicon. The molecular diagnostic test device can also include a detection surface on which the target amplicon is detectable by producing a visible signal, the detection surface being biased against the housing such that the visible signal is visible through the viewing area.

In accordance with some embodiments, the molecular diagnostic test device includes a support substrate within the housing. The support substrate supports a portion of the detection surface. The support substrate includes a biasing member suitable to apply a biasing force on the detection surface causing the detection surface to be biased against the housing. The detection surface is configured to retain a set of target amplicons within a single region to produce the visible signal. The viewing area can include a beveled edge and an opening defined by the housing. The beveled edge surrounds the opening. The visible signal from the detection surface is visible through the opening. The viewing area can be a window defined in the housing, with the visible signal from the detection surface being visible through the window.

In accordance with some embodiments, the housing includes a set of mounting protrusions. The support substrate can include a set of deformable mounting tabs. Each of the mounting tabs are in contact with a corresponding mounting protrusion to produce the biasing force, with the biasing member being a part off set of deformable mounting tabs. In some embodiments, the mounting tabs are located proximal to a bottom portion of the housing and are positioned to create a biasing force towards the top portion of the housing.

In accordance with some embodiments, the viewing area is defined by a top portion of the housing. The device includes a flow manifold, and the reaction module includes an amplification module and a detection module. The amplification module heats the input solution to amplify the target molecule. The detection module includes a detection housing defining a detection channel. The detection surface is within the detection channel. The biasing force urges an outer surface of the detection housing into contact with an inner surface of the housing to form a contact region surrounding the viewing area. A detection flow manifold is coupled to the detection module and defines a passageway through which the detection reagent is conveyed toward the detection channel. The detection module is coupled to receive the output from the amplification module and react within the detection channel the target amplicon with a detection reagent to produce the visible signal. The flow manifold includes a support substrate that supports a portion of the detection module. The support substrate includes a biasing member suitable to apply a biasing force on the detection module causing the detection surface to be biased against the housing.

In some embodiments, a molecular diagnostic test device includes a housing, a support substrate, and the set of modules. The housing has an outer wall defining a viewing area and contains the set of modules. The molecular diagnostic test device can include a support substrate located within the housing. The support substrate supports at least one of the modules and has a biasing member suitable to apply a biasing force on at least one of the modules. The set of modules includes a sample preparation module, an amplification module, and a detection module. The sample preparation module defines an input reservoir configured to receive a biological sample. The sample preparation module is configured to produce an amplification solution containing a target DNA molecule. The amplification module includes a flow member and a heater. The flow member defines a reaction volume configured to receive the amplification solution. The heater is configured to convey thermal energy into the reaction volume to amplify the target DNA molecule within the amplification solution to produce an output containing a target amplicon. The detection module defines a detection channel configured to receive the output from the amplification module and a reagent formulated to produce a signal that indicates a presence of the target amplicon from a detection surface. The biasing force urges the detection module in contact with an inner surface of the housing.

In accordance with some embodiments, the housing includes a set of mounting protrusions. The support substrate includes a set of deformable mounting tabs, each of the mounting tabs being in contact with a corresponding mounting protrusion to produce the biasing force. The biasing member includes one of the deformable mounting tabs. The viewing area is defined by a top portion of the housing. The mounting tabs are located proximal to a bottom portion of the housing and are positioned to create a biasing force towards the top portion of the housing.

In accordance with some embodiments, the molecular diagnostic test device can include a flow manifold coupled to the detection module. The flow manifold defines a passageway through which the detection reagent is conveyed toward the detection channel. The detection module is coupled to receive the output from the amplification module and react within the detection channel the target amplicon with a detection reagent to produce the signal. The flow manifold forms part of the support substrate. The deformable tabs deform to urge the detection module toward the upper housing. The detection module includes a detection housing defining the detection channel. The detection surface being within the detection channel. The biasing force urges an outer surface of the detection housing into contact with an inner surface of the housing to form a contact region surrounding the viewing area. The detection surface is configured to retain a target amplicon (or multiple target amplicons) within a single region to produce the signal. The viewing area includes a beveled edge and an opening defined by the housing. The beveled edge surrounds the opening. The signal from the detection surface is visible through the opening. The viewing area is a window defined in the housing, with the signal from the detection surface being visible through the window. The sample preparation module defines a sample input opening through which the biological sample can be loaded. The device includes a lid movably coupled to housing that covers the sample input opening. The lid is spaced apart from sample input opening to defining an air gap to the input reservoir.

In accordance with another embodiment, the molecular diagnostic test device can also include a housing having an opening. The molecular diagnostic test device can also include a sample preparation module within the housing. The sample preparation module defines a sample input opening aligned with the housing opening. The sample preparation also includes an input reservoir. The sample preparation module includes a retention screen positioned to separate the input reservoir into a first portion and a second portion. The sample input opening is within the first portion. The second portion contains a solid reagent. The sample preparation module is configured to receive a biological sample. The sample preparation module is configured to mix the biological sample with the solid reagent to form an input solution containing a target molecule.

In accordance with some embodiments, a reaction module is disposed within the housing. The reaction module is configured to receive the input solution from the second portion of the input reservoir and amplify the target molecule to produce an output containing a target amplicon. The retention screen defines multiple apertures that are sized to allow the biological sample to flow through the retention screen from the first portion of the input reservoir to the second portion of the input reservoir, while also maintaining the solid reagent within the second portion of the input reservoir. The input reservoir has a total volume and the retention screen is positioned within the sample preparation module such that a volume of the second portion of the input reservoir is greater than half of the total volume. The input reservoir has a height and the retention screen is positioned within the input reservoir at a screen distance below the sample input opening. A ratio of the screen distance to the height is between 0.3 and 0.6. The sample preparation module incudes a coupling protrusion within the input reservoir. The retention screen is coupled to the coupling protrusion. The retention screen is configured to allow gas bubbles to pass through the apertures. The solid reagent includes a lyophilized pellet. The lyophilized pellets include one or more of a reducing agent, positive control organism, reverse transcriptase enzymes, or salts. The reaction module includes an amplification module and a detection module. The amplification module heats the input solution to amplify the target molecule. The detection module defines a detection channel configured to receive the output from the amplification module and a reagent formulated to produce a visible signal that indicates a presence of the target amplicon from a detection surface.

In some embodiments, a molecular diagnostic test device includes a housing having an opening, a sample preparation module, and a reaction module. The sample preparation module and the reaction module are each within the housing. The sample preparation module defines a sample input opening aligned with the housing opening. The sample preparation also includes an input reservoir. The sample preparation module includes a retention screen positioned to separate the input reservoir into a first portion and a second portion. The sample input opening is within the first portion, and the second portion contains a solid reagent. The sample preparation module is configured to receive a biological sample and mix the biological sample with the solid reagent to form an input solution containing a target molecule.

In accordance with some embodiments, a reaction module is disposed within the housing. The reaction module is configured to receive the input solution containing the target molecule and amplify the target molecule to produce an output containing a target amplicon. The fluid source includes a fluid pump and an air reservoir with the fluid pump in fluid communication with the sample preparation module such that air from the air reservoir can be pumped to the sample preparation module. The air forms bubbles in the sample preparation module to agitate the biological sample and the reagent. In some embodiments, the pump is a piston pump including a piston that is an extended position within a syringe body to define the air reservoir prior to the biological sample being loaded into the input reservoir. The piston is configured to move in a first direction to force the air out of the syringe body and into the sample preparation module, the piston configured to move in a second direction opposite the first direction to pull the mixed biological sample and reagent into the reaction module. The sample preparation module includes a retention screen positioned to separate the input reservoir into a first portion and a second portion. The retention screen defines multiple apertures that are sized to allow the biological sample to flow through the retention screen from the first portion of the input reservoir to the second portion of the input reservoir, while also maintaining the solid reagent within the second portion of the input reservoir. The screen is configured to allow the air bubbles to pass through the apertures. The sample preparation module defines a sample input opening through which the biological sample can be loaded. In some embodiments, the molecular diagnostic test device further includes a lid movably coupled to housing that covers the sample input opening, the lid being spaced apart from sample input opening to define an air gap to the input reservoir allowing air to pass out of the input reservoir.

In accordance with some embodiments, a method for detecting a nucleic acid with a molecular diagnostic test device includes conveying a biological sample into a sample preparation module within the molecular diagnostic test device via an input opening. The biological sample is received into an input reservoir that contains a reagent. The input opening is covered with a lid coupled to the molecular diagnostic test device. The molecular diagnostic test device is actuated causing the molecular diagnostic test device to: drive a fluid into the input reservoir to mix the biological sample and the reagent within the input reservoir, convey the mixed biological sample out of the input reservoir and into a reverse transcription flow path, heat the biological sample via a heater of the sample preparation module within the reverse transcription flow path to produce an input solution containing cDNA, heat the input solution within a reaction volume within the molecular diagnostic test device to amplify the cDNA within the input solution thereby producing an output solution containing a target amplicon, and react within the molecular diagnostic test device each of (i) the output solution and (ii) a reagent formulated to produce a signal that indicates a presence of the target amplicon within the output solution. The result associated with the signal is read. In some embodiments, the fluid is air, and the air is driven into the input reservoir by a pump causing air bubbles to form in the biological sample and agitate the biological sample and the reagent.

In accordance with some embodiments, the method for detecting nucleic acid can include mixing the input solution containing cDNA with a second reagent before the input solution is heated within the reaction volume to amplify the cDNA. The biological sample can be loaded in a first direction within a flow path into the input reservoir and the fluid is injected into the input reservoir from a second direction along the flow path. In some embodiments, the pump can include a piston and a syringe body, the piston within the syringe body to contain a volume of air prior to conveying the biological sample. The piston is moved in a first piston direction in response to actuation of the molecular diagnostic test device to drive air into the input reservoir along the reverse transcription flow path causing bubbles in the input reservoir, and the piston is moved in a second piston direction to convey the mixed biological sample into the reverse transcription flow path. The sample preparation module defines a sample input opening through which the biological sample can be conveyed. The molecular diagnostic test device includes a lid movably coupled to housing that covers the sample input opening. The lid is spaced apart from sample input opening to define an air gap to the input reservoir. The air driven into the input reservoir can pass through the air gap and out of the input reservoir. In some embodiments, the molecular diagnostic test device includes a control module, the control module including the switch and a processor, the processor being configured to control transmission of power to the pump of the molecular diagnostic test device for driving air to the input reservoir and pulling the mixed biological sample out of the input reservoir.

In some embodiments, a molecular diagnostic test device includes a housing, a sample preparation module within the housing, a fluid source within the housing, and a reaction module within the housing. The sample preparation module defines a sample input opening through which a biological sample can be loaded and a reservoir containing a reagent. The fluid source is in fluid communication with the reservoir such that during operation of the molecular diagnostic test device, fluid from the fluid source is injected into the reservoir to mix the biological sample and the reagent within the reservoir to form a solution containing a target molecule for amplification. The reaction module is configured to receive the solution containing the target molecule and amplify the target molecule to produce an output containing a target amplicon.

In some embodiments, the molecular diagnostic test device includes a detection module coupled to the reaction module, the detection module configured to produce a signal in response to a presence of the target amplicon within the amplification solution being detected.

In some embodiments, the fluid source includes a fluid pump and an air reservoir within the fluid pump in fluid communication with the reservoir such that air from the air reservoir can be pumped to the reservoir. In some embodiments, the fluid pump is a piston pump including a piston that is an extended position within a syringe body to define the air reservoir. The piston is configured to move in a first direction to force the air out of the syringe body and into the sample preparation module. The piston configured to move in a second direction opposite the first direction to pull the biological sample and reagent into the reaction module.

In some embodiments, the reagent is a second reagent (e.g., a PCR reagent). The reservoir is a mixing reservoir that is in communication with an input reservoir that contains a first reagent. The fluid pump can optionally be in fluid communication with the mixing reservoir such that the fluid pump forces air through the mixing reservoir in response to the molecular diagnostic test device being in a first configuration and the fluid pump forces air through the input reservoir in response to the molecular diagnostic test device being in a second configuration.

In some embodiments, the reservoir is defined by a mixing assembly housing that has a first end portion and a second end portion. The mixing assembly housing is coupled within the housing such that when a base of the housing is on a testing surface, the second end portion of the mixing housing is above the first end portion of the mixing housing. The first end portion of the mixing housing defines a mixing input opening and the second end portion of the mixing housing defining a mixing output opening. In such embodiments, the molecular diagnostic test device also includes a valve configured to selectively fluidically couple the air reservoir to the mixing reservoir via the mixing input opening. The valve is also configured to selectively fluidically couple the mixing reservoir to a vent via the mixing output opening.

In some embodiments, a method of detecting a nucleic acid using a molecular diagnostic test device includes conveying a biological sample into a sample preparation module within the molecular diagnostic test device via an input opening. The test device is then actuated, cause the test device to convey the biological sample into a reverse transcription flow path. Actuation also causes the test device to heat the biological sample via a heater of the sample preparation module within the reverse transcription flow path to produce an input solution containing cDNA. The input solution containing cDNA is then conveyed into a reservoir that contains a reagent. A fluid is driven into the reservoir to mix the input solution and the reagent within the reservoir to produce an amplification solution. Actuation also causes the test device to heat the amplification solution within a reaction volume within the molecular diagnostic test device to amplify the cDNA within the amplification solution thereby producing an output solution containing a target amplicon. The device then reacts within the molecular diagnostic test device each of (i) the output solution and (ii) a reagent formulated to produce a signal that indicates a presence of the target amplicon within the output solution. The method further includes reading a result associated with the signal.

In accordance with some embodiments, the reagent is a first reagent, the reservoir is a mixing reservoir and the biological sample is received into an input reservoir that contains a second reagent. Actuating the molecular diagnostic test device further causes the molecular diagnostic test device to: mix the second reagent with the biological sample; and convey the biological sample out of the input reservoir and into the reverse transcription flow path. In some embodiments, the fluid is air, and the air is driven into the mixing reservoir by a fluid pump thereby using air bubbles within the biological sample to agitate the biological sample and the reagent. Actuating the molecular diagnostic test device further causes the molecular diagnostic test device to drive, via the fluid pump, the fluid into the input reservoir to mix the biological sample and the reagent within the reservoir. The biological sample is loaded in a first direction within a flow path into the input reservoir and the fluid is driven into the input reservoir from a second direction along the flow path. The fluid pump is in fluid communication with the mixing reservoir and the input reservoir such that the fluid pump forces the air through the mixing reservoir in response to the molecular diagnostic test device in a first configuration. The fluid pump forces air through the input reservoir in response to the molecular diagnostic test device being in a second configuration. The fluid pump is in serial fluid communication with the mixing reservoir and the input reservoir with the mixing reservoir positioned between the fluid pump and the input reservoir.

In some embodiments, the pump includes a piston and a syringe body. The pump contains a volume of the air prior to conveying the biological sample. The piston is moved in a first piston direction in response to actuation of the molecular diagnostic test device to drive the air into the input reservoir along the reverse transcription flow path causing bubbles in the input reservoir. The piston is moved in a second piston direction to convey the biological sample into the reverse transcription flow path. The molecular diagnostic test device further includes a valve in fluid communication with the mixing reservoir. The valve is configured to vent an inlet into the mixing reservoir while the piston is moved causing air to be pulled through the vent into the reservoir. The molecular diagnostic test device includes a control module. The control module includes a switch and a processor. The processor is configured to control transmission of power to the pump of the molecular diagnostic test device for driving air to at least one of the input reservoir or the mixing reservoir.

In some embodiments a molecular diagnostic test device includes a housing and a sample preparation module within the housing. The sample preparation module defines an input reservoir configured to receive a sample quantity of a biological sample containing a target molecule. The input reservoir in fluid communication with a mixing reservoir containing a reagent quantity of a PCR reagent. A ratio of sample quantity to the reagent quantity is between about 3 to 1 and 7 to 1. The ratio of sample quantity to the reagent quantity is about 5:1. For example, if the sample quantity is about 650 μl, then the PCR reagent quantity can be about 130 μl. The sample preparation module is configured to produce an amplification solution containing a portion of the biological sample and the PCR reagent. A reaction module is disposed within the housing. The reaction module is configured to receive the amplification solution containing the target molecule and the PCR reagent and amplify the target molecule to produce an output containing a target amplicon. The molecular diagnostic test device is configured to produce a signal in response to a presence of the target amplicon being detected within the amplification solution.

In accordance with some embodiments, the portion of the sample quantity is a first portion. The sample preparation module is configured to produce a drive solution containing at least a second portion of the biological sample. A ratio of an amount the PCR reagent within the amplification solution is greater than a ratio of an amount of PCR reagent within the drive solution. The molecular diagnostic test device includes a detection module fluidically coupled to the reaction module. The detection module is configured to produce the signal. The molecular diagnostic test device is configured such that the drive solution exerts a force onto the amplification solution to convey the amplification solution from the reaction module to the detection module. The sample preparation module is filterless. The sample quantity input in to the device is between about 500-700 μl; and the amplification solution is between about 100-200 μl. The detection module includes a detection module volume. The sample preparation module and the reaction module are connected via a fluid pathway therebetween. The fluid pathway includes a volume approximately equal to the detection module volume. A volume of the amplification solution is approximately equal to the detection module volume.

In accordance with some embodiments a method for detecting a nucleic acid using a molecular diagnostic test device is provided. The method includes conveying a biological sample into a sample preparation module within the molecular diagnostic test device via an input opening. The method also includes actuating the molecular diagnostic test device. The actuating also causes the molecular diagnostic test device to convey the biological sample into a reverse transcription flow path. The actuating also causes the molecular diagnostic test device to heat the biological sample via a heater of the sample preparation module within the reverse transcription flow path to produce an input solution. The actuating also causes the molecular diagnostic test device to convey the input solution into a reservoir that contains a reagent. The actuating also causes the molecular diagnostic test device to drive a fluid into the reservoir to mix the input solution and the reagent within the reservoir to produce an amplification solution. The actuating also causes the molecular diagnostic test device to heat the amplification solution within a reaction volume within the molecular diagnostic test device thereby producing an output solution containing a target amplicon. The actuating also causes the molecular diagnostic test device to react within the molecular diagnostic test device each of (i) the output solution and (ii) a reagent formulated to produce a signal that indicates a presence of the target amplicon within the output solution. The method also includes reading a result associated with the signal.

A molecular diagnostic test device includes a housing and a sample preparation module within the housing. The sample preparation module defines an input reservoir configured to receive a sample quantity of a biological sample containing a target molecule. The input reservoir is in fluid communication with a mixing reservoir containing a reagent of a PCR reagent. The sample preparation module is configured to produce an amplification solution containing a portion of the biological sample and the PCR reagent from a first portion of the biological sample. The sample preparation module is configured to produce a drive solution in fluid communication with the amplification solution from a second portion of the biological sample. The drive solution includes a lower reagent concentration than the amplification solution. A reaction module is disposed within the housing. The reaction module is configured to receive the amplification solution containing the target molecule and the PCR reagent and amplify the target molecule to produce an output containing a target amplicon. The molecular diagnostic test device is configured to produce a signal in response to a presence of the target amplicon being detected within the amplification solution.

In some embodiments, the molecular diagnostic test device includes a housing and a sample preparation module within the housing. The sample preparation module defines an input reservoir configured to receive a first quantity of a biological sample containing a target molecule. The input reservoir in fluid communication with a mixing reservoir containing a PCR reagent. The sample preparation module is configured to produce an amplification solution with the PCR reagent and the target molecule. A reaction module is disposed within the housing. The reaction module is configured to receive the amplification solution containing the target molecule and the PCR reagent and amplify the target molecule producing an output containing a target amplicon. The sample preparation module and the reaction module are connected via a fluid pathway. The device also includes a detection module producing a signal in response to a presence of the target amplicon being detected. The detection module includes a detection module volume. The fluid pathway includes a volume approximately equal to the detection module volume.

### BRIEF DESCRIPTION OF THE DRAWINGS

FIG. 1 is a schematic illustration of a molecular diagnostic test device, according to an embodiment.

FIG. 2 is a schematic illustration of a molecular diagnostic test device, according to an embodiment, in a first configuration.

FIGS. 3-5 are schematic illustrations of the molecular diagnostic test device shown FIG. 2, in a second configuration (FIG. 3), a third configuration (FIG. 4), and a fourth configuration (FIG. 5).

FIG. 6 is a flow chart of a method of detecting a nucleic acid, according to an embodiment.

FIG. 7A is a diagram illustrating an enzyme linked reaction, according to an embodiment, resulting in the production a signal.

FIG. 7B is a schematic illustration of a molecular diagnostic test device, according to an embodiment.

FIGS. 8 and 9 are a perspective view and a top view, respectively, of a molecular diagnostic test device, according to an embodiment.

FIGS. 10 and 11 are exploded views of the molecular diagnostic test device shown in FIGS. 8 and 9.

FIGS. 12 and 13 are a front perspective view (FIG. 12) and a rear perspective view (FIG. 13) of the molecular diagnostic test device shown in FIGS. 8 and 9, with the housing removed to show the modules therein.

FIG. 14 is an exploded perspective view of the housing assembly of the molecular diagnostic test device shown in FIGS. 8 and 9.

FIG. 15 is a bottom perspective view of the top housing of the molecular diagnostic test device shown in FIGS. 8 and 9.

FIG. 16 is side cross-sectional views taken along line W-W in FIG. 9 of the molecular diagnostic test device shown in FIGS. 8 and 9.

FIGS. 17A, 17B, and 18 are a front perspective view (FIG. 17A), a rear perspective view (FIG. 17B), and a bottom perspective view (FIG. 18) of the lid of the molecular diagnostic test device shown in FIGS. 8 and 9.

FIGS. 19 and 20 are a top perspective view (FIG. 19) and a bottom perspective view (FIG. 20) of the flexible plate of the molecular diagnostic test device shown in FIGS. 8 and 9.

FIGS. 21 and 22 are side cross-sectional views taken along line X-X in FIG. 9, showing the molecular diagnostic test device in a first (pre-actuated) configuration and a second (post-actuated) configuration, respectively.

FIGS. 23 and 24 are a top perspective view (FIG. 23) and a bottom perspective view (FIG. 24) of the deformable support member of the molecular diagnostic test device shown in FIGS. 8 and 9.

FIGS. 25 and 26A are a perspective view (FIG. 25) and a top view (FIG. 26A) of the sample preparation module of the molecular diagnostic test device shown in FIGS. 8 and 9.

FIGS. 26B and 27 are a cross-sectional view (FIG. 26B) and a cross-sectional perspective view (FIG. 27) of the sample preparation module shown in FIGS. 25 and 26.

FIG. 28 is an exploded view of the sample preparation module shown in FIGS. 25 and 26.

FIG. 29 is a cross-sectional view taken along line X-X in FIG. 26A of the mixing assembly of the sample preparation module shown in FIGS. 25 and 26A.

FIG. 30 is a top view of a flow member of the amplification module of the molecular diagnostic test device shown in FIGS. 8 and 9.

FIG. 31 is an exploded view of the detection module of the molecular diagnostic test device shown in FIGS. 8 and 9.

FIGS. 32 and 33 are a top perspective view (FIG. 32) and a bottom perspective view (FIG. 33) of the reagent module of the molecular diagnostic test device shown in FIGS. 8 and 9.

FIG. 34 is a front perspective view of rotary valve assembly of the molecular diagnostic test device shown in FIGS. 8 and 9.

FIGS. 35-40 are front views of the rotary valve assembly shown in FIG. 46 showing the valve disc in each of six different operational configurations.

FIGS. 41A-41C are perspective views of the molecular diagnostic device shown in FIGS. 8 and 9 in various stages of operation, according to an embodiment.

FIG. 42 shows the concentration factor of a red dye and a blue dye in a solution across 20 aliquots when mixed under a forced air in the preparation chamber.

FIG. 43 is a schematic illustration of a molecular diagnostic test device, according to an embodiment.

FIGS. 44-50 are perspective, right side, left side, rear, front, top, and bottom views, respectively, of one embodiment of an ornamental design of a molecular test device.

FIG. 51A is a schematic illustration of a portion of a method within a molecular diagnostic test device, according to an embodiment.

FIG. 51B is a schematic illustration of another portion of the method within the molecular diagnostic test device of FIG. 51A.

FIG. 51C is a schematic illustration of another portion of the method within the molecular diagnostic test device of FIG. 51A.

FIG. 51D is a schematic illustration of another portion of the method within the molecular diagnostic test device of FIG. 51A.

FIG. 51E is a schematic illustration of another portion of the method within the molecular diagnostic test device of FIG. 51A.

FIG. 52 is a flow chart illustrating a method for detecting a nucleic acid using a molecular diagnostic test device, according to an embodiment.

FIG. 53A is a perspective view of a mixing reservoir of a molecular diagnostic test device, according to an embodiment.

FIG. 53B is a perspective view of the mixing reservoir of FIG. 53A.

FIG. 53C is a cross-sectional view taken along line Z-Z in FIG. 52B of the mixing reservoir of FIGS. 53A and 53B.

FIG. 54 is a flow chart illustrating a method for detecting a nucleic acid using a molecular diagnostic test device.

FIG. 55A shows the concentration of a solution mixed under a forced air method in a mixing reservoir across multiple aliquots.

FIG. 55B shows the concentration of a solution mixed under a forced air method in a mixing reservoir across multiple aliquots.

FIG. 55C shows the concentration of a solution mixed under a forced air method in a mixing reservoir across multiple aliquots.

FIG. 55D shows the concentration of a solution when unmixed in a mixing reservoir across multiple aliquots for a controlled comparison to FIGS. 53A-53C.

### DETAILED DESCRIPTION

In some embodiments, an apparatus is configured for a disposable, portable, single-use, inexpensive, molecular diagnostic approach. The apparatus can include one or more modules configured to perform high quality molecular diagnostic tests, including, but not limited to, sample preparation, nucleic acid amplification (e.g., via polymerase chain reaction, isothermal amplification, or the like), and detection.

In some embodiments, the devices described herein are stand-alone devices that include all necessary substances, mechanisms, and subassemblies to perform any of the molecular diagnostic tests described herein. Such stand-alone devices do not require any external instrument to manipulate the biological samples, and only require connection to a power source (e.g., a connection to an A/C power source, coupling to a battery, or the like) to complete the methods described herein. For example, the device described herein do not require any external instrument to heat the sample, agitate or mix the sample, to pump (or move) fluids within a flow member, or the like. Rather, the embodiments described herein are fully-contained and upon loading a biological sample and being coupled to a power source, the device can be actuated to perform the molecular diagnostic tests described herein. In some embodiments, the method of actuating the device can be such that the device is a CLIA-waived device and/or can operate in accordance with methods that are CLIA waived.

As used in this specification and the appended claims, the term “reagent” includes substance that is used in connection with any of the reactions described herein. For example, a reagent can include an elution buffer, a PCR reagent, an enzyme, a substrate, a wash solution, a blocking solution, or the like. A reagent can include a mixture of one or more constituents. A reagent can include such constituents regardless of their state of matter (e.g., solid, liquid or gas). Moreover, a reagent can include the multiple constituents that can be included in a substance in a mixed state, in an unmixed state and/or in a partially mixed state. A reagent can include both active constituents and inert constituents. Accordingly, as used herein, a reagent can include non-active and/or inert constituents such as, water, colorant or the like.

The term “nucleic acid molecule,” “nucleic acid,” or “polynucleotide” may be used interchangeably herein, and may refer to deoxyribonucleic acid (DNA) or ribonucleic acid (RNA), including known analogs or a combination thereof unless otherwise indicated. Nucleic acid molecules to be profiled herein can be obtained from any source of nucleic acid. The nucleic acid molecule can be single-stranded or double-stranded. In some cases, the nucleic acid molecules are DNA The DNA can be mitochondrial DNA, complementary DNA (cDNA), or genomic DNA. In some cases, the nucleic acid molecules are genomic DNA (gDNA). The DNA can be plasmid DNA, cosmid DNA, bacterial artificial chromosome (BAC), or yeast artificial chromosome (YAC). The DNA can be derived from one or more chromosomes. For example, if the DNA is from a human, the DNA can be derived from one or more of chromosomes 1, 2, 3, 4, 5, 6, 7, 8, 9, 10, 11, 12, 13, 14, 15, 16, 17, 18, 19, 20, 21, 22, X, or Y. In some cases, the nucleic acid molecules are RNA can include, but is not limited to, mRNAs, tRNAs, snRNAs, rRNAs, retroviruses, small non-coding RNAs, microRNAs, polysomal RNAs, pre-mRNAs, intronic RNA, viral RNA, cell free RNA and fragments thereof. The non-coding RNA, or ncRNA can include snoRNAs, microRNAs, siRNAs, piRNAs and long nc RNAs. The source of nucleic acid for use in the devices, methods, and compositions described herein can be a sample comprising the nucleic acid.

Unless indicated otherwise, the terms apparatus, diagnostic apparatus, diagnostic system, diagnostic test, diagnostic test system, test unit, and variants thereof, can be interchangeably used.

The methods described herein can be performed on any suitable molecular diagnostic device, such as any of the diagnostic devices shown and described herein or in International Patent Publication No. WO2016/109691, entitled “Devices and Methods for Molecular Diagnostic Testing,” International Patent Publication No. WO2017/185067, entitled “Printed Circuit Board Heater for an Amplification Module,” International Patent Publication No. WO2018/005710, entitled “Devices and Methods for Detection of Molecules Using a Flow Cell,” and International Patent Publication No. WO2018/005870, entitled “Devices and Methods for Nucleic Acid Extraction,” International Patent Publication No. WO2020/223257A1 entitled “Molecular Diagnostic Devices with Digital Detection Capability and Wireless Connectivity,” International Patent Publication No. WO2021/138544A1 entitled “Devices and Methods for Antibiotic Susceptibility Testing,” U.S. Patent Publication No. US2019/0169677, entitled “Portable Molecular Diagnostic Device and Methods for Detection of Target Viruses.” each of which is incorporated herein by reference in its entirety.

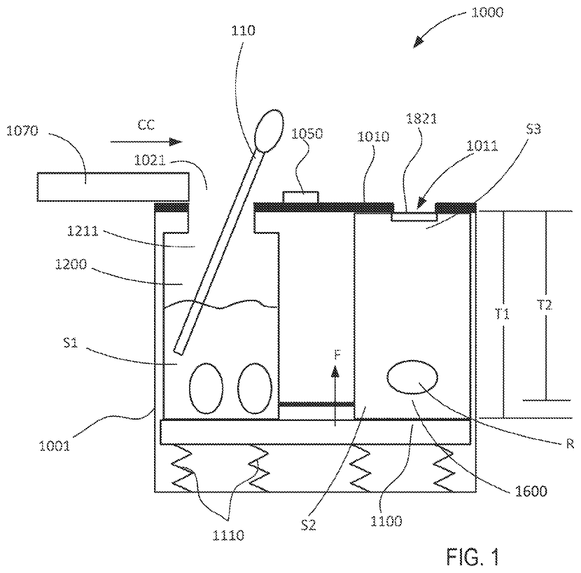

FIG. 1 is a schematic illustrations of a molecular diagnostic test device 1000 (also referred to as a “test device” or “device”), according to an embodiment. The test device 1000 is configured to manipulate biological sample to produce one or more output signals associated with a target cell, according to the various systems and the methods described herein. In some embodiments, the test device 1000 can be an integrated device that is suitable for use within a point-of-care setting (e.g., doctor's office, pharmacy or the like), decentralized test facility, or at the user's home. Similarly stated, in some embodiments, the modules of the device, described below, are contained within a single housing such that the test device can be fully operated without any additional instrument, docking station, or the like. Further, in some embodiments, the device 1000 can have a size, shape and/or weight such that the device 1000 can be carried, held, used and/or manipulated in a user's hands (i.e., it can be a “handheld” device). In some embodiments, the test device 1000 can be a self-contained, single-use device.

In some embodiments, the device 1000 (and any of the devices shown and described herein) can be a CLIA-waived device and/or can operate in accordance with methods that are CLIA waived. Similarly stated, in some embodiments, the device 1000 (and any of the other devices shown and described herein) is configured to be operated in a sufficiently simple manner and can produce results with sufficient accuracy to pose a limited likelihood of misuse and/or to pose a limited risk of harm if used improperly. In some embodiments, the device 1000 (and any of the other devices shown and described herein), can be operated by a user with minimal (or no) scientific training, in accordance with methods that require little judgment of the user, and/or in which certain operational steps are easily and/or automatically controlled. In some embodiments, the molecular diagnostic test device 1000 can be configured for long term storage in a manner that poses a limited likelihood of misuse (spoilage of the reagent(s), expiration of the reagents(s), leakage of the reagent(s), or the like). In some embodiments, the molecular diagnostic test device 1000 is configured to be stored for up to about 36 months, up to about 32 months, up to about 26 months, up to about 24 months, up to about 20 months, up to about 18 months, up to 12 months, up to 6 months, or any values there between.

The test device 1000 includes a housing 1001, a lid 1070, an actuator 1050, a sample preparation module 1200, a reaction module 1600, and a detection surface 1821. In some embodiments, the test device 1000 can include any other components or modules described herein, such as, for example, an amplification module, a detection module, a reagent module that contains on-board reagents (e.g., the reagent module 3700)), a rotary valve (e.g., to control flow of reagents and/or sample, such as the valve 3300), or a fluid transfer module (e.g., the fluid drive module 3400). The housing 1001 can be any structure within which the sample preparation module 1200 or other components are contained (or partially contained) to form an integrated device for sample preparation and/or molecular testing. The housing 1001 can be a monolithically constructed housing or can include multiple separately constructed members that are later joined together to form the housing 1001 or be contained therein.

As shown in FIG. 1, the housing 1001 includes an external wall 1010 that defines one or more external features. For example, the housing 1001 defines an input opening 1021 through which a biological sample S1 can be conveyed into the sample preparation module 1200. The housing 1001 also defines a viewing area 1011 for seeing a visual display of test results or operation. The viewing area can include an opening through the external wall 1010 of the housing 1001. In some embodiments, the viewing area can include a window or clear material through which test results can be viewed. The viewing area can include any suitable features to enhance viewing. For example, in some embodiments, the viewing area includes a beveled edge that surrounds (or partially surrounds) the opening. In some embodiments, the housing includes a mask portion (e.g., having contrasting colors or features) that surrounds at least a portion of the opening. The mask portion can be configured to enhance visibility of the detection surface 1821 through the viewing area or detection opening.

The sample preparation module 1200 is configured to manipulate the biological sample S1 for further diagnostic testing. For example, in some embodiments, the sample preparation module 1200 can extract target molecules (e.g., nucleic acid) from the biological sample S1 and can produce an input solution S2 that is conveyed into the reaction module 1600. Referring to FIG. 1, the sample preparation module 1200 defines a sample input reservoir 1211 that receives a biological sample S1. In some embodiments, the biological sample S1 can be conveyed into the device by a sample transfer device 110. The sample transfer device 110 can be any suitable device, such as a pipette or other mechanism configured can be used to aspirate or withdraw the sample S1 from a sample cup, container or the like, and then deliver a desired amount of the sample via the opening 1021. After the sample S1 is loaded via the opening 1021, the lid 1050 can be closed (as shown by the arrow CC). The sample preparation module 1200 can include any components as described herein to manipulate the biological sample S1 for further diagnostic testing and/or to produce a solution for detection of a target molecule (e.g., nucleic acid). For example, in some embodiments, the sample preparation module 1200 can include one or more heaters, one or more chambers within which the biological sample S1 can be manipulated, one or more mixing reservoirs, and/or certain on-board reagents (e.g., a lysing buffer, an RT enzyme, a control substance, or the like). In some embodiments, the sample preparation module 1200 can function merely as a sample holding or mixing reservoir. For example, in some embodiments, the sample preparation module 1200 can contain the desired amplification reagents to facilitate a desired amplification according to any of the methods described herein. In other embodiments, the sample preparation module 1200 is configured to extract nucleic acid molecules from the biological sample S1 and can produce an input solution S2 (see FIG. 1) that is conveyed into the reaction module 1600.

The reaction module 1600 defines an internal reservoir (e.g., a reaction chamber or reaction volume) that receives the input solution from the sample preparation module 1200 and amplifies a target molecule therein to produce an output containing a target amplicon. The reaction volume can be formed from any suitable structure that defines a volume or a series of volumes within which the input solution S2 can flow and/or be reacted to produce a solution for subsequent detection. Thus, the reaction module 1600 can function as an amplification module, a lysis module, a detection module, or any other module within which a reaction can occur to facilitate detection of the target polynucleotide sequence. In some embodiments, the reaction module 1600 can amplify the target nucleic acid molecules therein to produce an output detection solution that contains a target amplicon (or multiple target amplicons) to be detected. For example, in some embodiments, the reaction module 1600 (or any of the reaction modules or amplification modules described herein) can be similar to the amplification modules shown and described in U.S. Patent Publication No. 2017/0304829, entitled “Printed Circuit Board Heater for an Amplification Module,” which is incorporated herein by reference in its entirety. In other embodiments, the amplification module 1600 (or any of the amplification modules described herein) can be similar to the amplification modules shown and described in International Patent Publication No. WO2016/109691, entitled “Devices and Methods for Molecular Diagnostic Testing.”

In accordance with various embodiments, the reaction module can include one or more components of an amplification modules and/or one or more components of a detection module. For example, the reaction module 1600 can include a heater that can heat the input solution to perform any of the amplification operations as described herein. For example, in some embodiments, the reaction module 1600 (or any of the reaction modules or amplification modules described herein) can be similar to or include components of the amplification modules shown and described in U.S. Patent Publication No. 2017/0304829, entitled “Printed Circuit Board Heater for an Amplification Module,” which is incorporated herein by reference in its entirety. In other embodiments, the reaction module 1600 (or any of the reaction modules or amplification modules described herein) can be similar to include components of the amplification modules shown and described in International Patent Publication No. WO2016/109691, entitled “Devices and Methods for Molecular Diagnostic Testing.”

Although the amplification modules described herein are generally described as performing a thermal cycling operation on the input solution S2, in other embodiments, the reaction module 1600 (and any of the amplification modules described herein) can perform any suitable thermal reaction to amplify nucleic acids within the solution. In some embodiments, the reaction module 1600 (and any of the amplification modules described herein) can perform any suitable type of isothermal amplification process, including, for example, Loop Mediated Isothermal Amplification (LAMP), Nucleic Acid Sequence Based Amplification (NASBA), which can be useful to detect target RNA molecules, Strand Displacement Amplification (SDA), Multiple Displacement Amplification (MDA), Ramification Amplification Method (RAM), or any other type of isothermal process.

In accordance with some embodiments, the reaction module 1600 is configured to react the biological sample (identified as the processed solution S3 below) with one or more reagents to cause production of one or more assay signals to indicate presence of the target polynucleotide sequence. Although the biological sample is identified as a portion (i.e., S3) of the initial biological sample (i.e., S1) that has been processed, reacted or prepared within the sample preparation module 1200 and the reaction module 1600, in other embodiments, the portion of the biological sample that is further reacted within the reaction module 1600 can be any suitable portion of the initial biological sample S1. As described herein, the presence of the target polynucleotide sequence can indicate the presence of a target organism, whether the target organism is susceptible to a course of treatment, whether the target organism is resistant to a course of treatment, or other characteristics of the target organism. The reaction module 1600 can define a detection volume within which the biological sample and one or more reagents (see reagent R discussed below) can be reacted. The reacting can be performed by combining (e.g., mixing) the reagent R and the biological sample S3 within the reaction module 1600, by introducing each of the reagent R and the biological sample S3 into the reaction module 1600 (either at the same time or in a sequential manner), by conveying the biological sample S3 into the reaction module 1600, within which the reagent R has been stored for use, or any other suitable method for producing the desired reaction. In some embodiments, the reaction module 1600 can include one or more detection surfaces (e.g., detection surface 1821 described below) to which one or more probes are attached. As described herein, such probes can be designed to permit annealing or hybridization of a target amplicon with sufficient specificity to permit detection of the presence (or absence) of a target amplicon indicating the presence of the target polynucleotide sequence. In other embodiments, the detection module can include one or more detection chambers in which different reagents or probes can be combined or reacted with the biological sample to produce a series of assay signals.

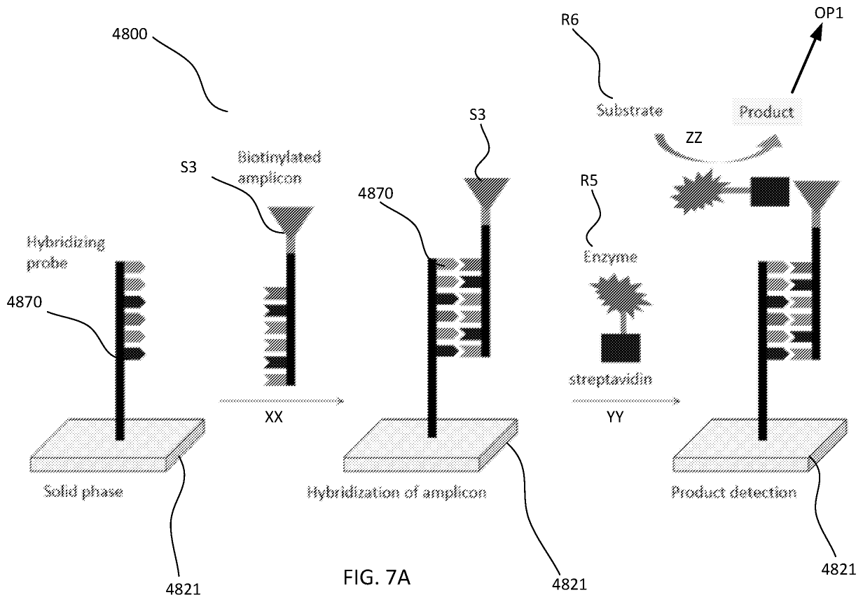

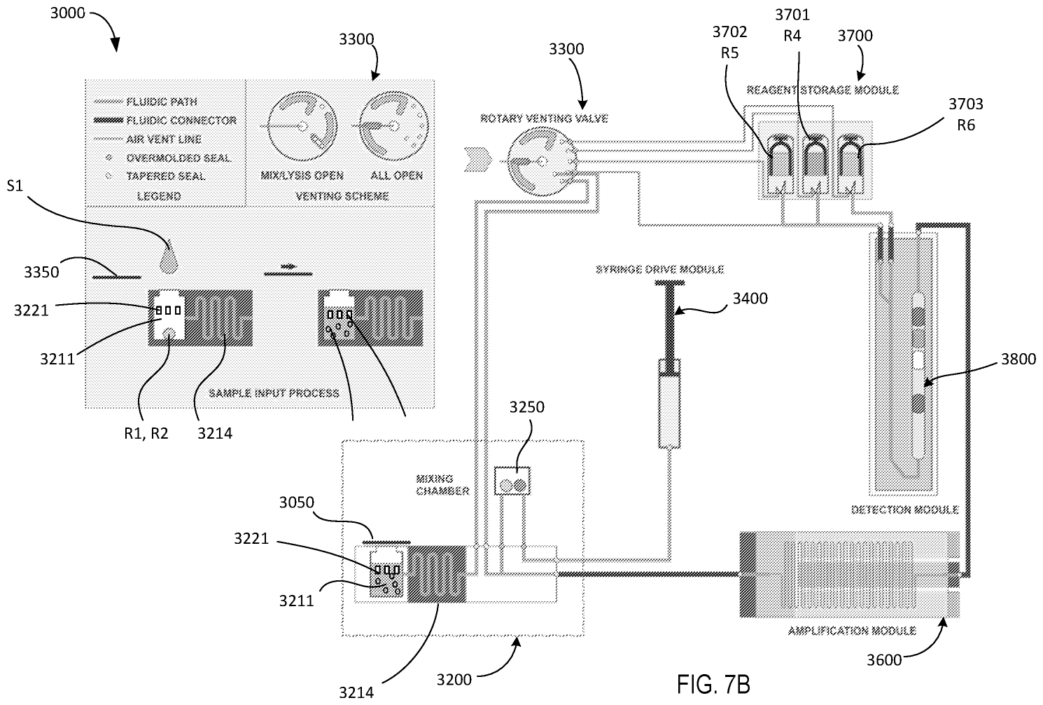

The detection surface 1821 is a surface from which the target amplicon is detectable by producing a visible signal. Specifically, as described herein, the output solution from (or within) the amplification module 1600 can be reacted with one or more reagents to produce a signal (or output) to indicate presence or absence of a target organism in the biological sample S1. In some embodiments, the detection surface 1821 can retain the target amplicon produced during amplification within a single region to produce a visible signal. In some embodiments, the detection surface 1821 can include a series of capture probes to which the target amplicon can be bound when the output solution is in contact with the detection surface 1821 (e.g., according to the enzyme-linked reaction process shown and described with reference to FIG. 7A). The capture probes can be any suitable probe of the types described herein formulated to capture or bind to the target amplicon.

In some embodiments, the detection surface 1821 is within the reaction module 1600. For example, in some embodiments, a portion of a wall that defines the reaction volume can also include the detection surface 1821. In this manner, the amplification reaction and reaction to produce the signal can occur within the same module and/or location (i.e., the reaction module 1600). In other embodiments, the detection surface 1821 can be spaced apart from (or outside of) the reaction module 1600. For example, in some embodiments the device 1000 can include a separate detection module (not shown) that defines a detection channel and includes the detection surface 1821 within the detection channel. The detection channel is in (or can be placed in) fluid communication with the reaction module 1600. In this manner, the output solution containing the target amplicon can be conveyed into the detection channel and across the detection surface 1821.

As shown in FIG. 1, one or more of the modules or components of the test device 1000 are biased against one or more surfaces of the housing 1001. Specifically, the detection surface 1821 is biased against the external wall 1010 of the housing such that the visible signal produced at the detection surface 1821 is visible through the viewing area 1011. The external wall 1010 can include (as shown in this example) an upper wall, thus the detection surface 1821 is urged towards the upper wall of the device. The biasing may be applied against one or more of the modules within the housing 1001 or one or more components of the modules in any suitable manner. For example, one of the modules within the housing 1001 can include (or be supported by) a support substrate 1100. The support substrate 1100 engages one or more biasing members 1110 such that the support substrate 1100 is biased in one direction in the housing. In this example, the support substrate 1100 is biased towards the external wall 1010 that defines the viewing area 1011. Thus, the support substrate 1100 can apply a biasing force, via the biasing member 1110, such that components of one or more modules are pressed against the housing wall 1010. Specially, the biasing force causes the detection surface to be biased against the housing 1001. In this manner, the biasing force (or preload) maximizes the viewing angle of the detection surface 1821 by ensuring that the detection surface is as close to the detection opening as possible. This arrangement allows for the detection surface 1821 to be close to the detection opening 1011 regardless of the manufacturing variability in dimensions of the components within the device 1000. In situations in which the detection surface is spaced apart from of the upper housing, the viewing angle is negatively affected. For example, in such situations shadows can be cast on the detection surface 1821 due to the gap. Thus, by eliminating part-to-part variability, consistency of test results can be improved. In some embodiments, the viewing area 1011 is defined by a beveled opening in the housing wall 1010, further reducing negative viewing effects of the detection surface (e.g., shading).

In some embodiments, the biasing force urges an outer surface of the module or component into contact with an inner surface of the housing wall 1010 to form a contact region. surrounding the viewing area. For example, the detection surface 1821 can be biased against the housing wall 1010 at or near the viewing area 1011 (as described above) forming a contact region around the viewing area 1011. In addition to enhancing viewing, having a tight press between the detection surface 1821 and the external wall 1010 reduces or prevents leakage through the viewing area 1011.

The support substrate 1100 can be any suitable structure within the housing 1001. In some examples, the support substrate 1100 can form a component of any of the modules described herein (e.g., a manifold having flow channels 3035 discussed below). In other examples, the support substrate 1100 can be a separate component (e.g., a supporting structural component) that extends between different modules or supports a single module. As an example, FIG. 1 illustrates the support substrate 1100 supporting both the 1200 and the reaction module 1600 (as well as the detection surface 1821). In other examples, however, the support substrate 1100 can support just the sample preparation module 1200. Alternatively, the support substrate 1100 can support just the reaction module 1600. Additionally or alternately, the support substrate 1100 can support other modules.

The biasing member 1110 includes any suitable structure that applies a biasing force between two components such as, the housing, a module, and/or the support substrate. In some examples, the biasing member 1110 can include one or more springs (e.g., compression, extension, torsion, constant force, Belleville, drawbar, volute, flat, gas, etc.). In some examples, the biasing member 1110 can be integral to one or more components of the device 1000. In such examples, the biasing member 1110 can be a molded flexible protrusion from the support substrate such as a tab (e.g., see biasing mechanism 3110 below). Moreover, although the device 1000 is shown as including multiple biasing members, in other embodiments, a device can include only a single biasing member.

In some embodiments, the housing 1001 (or any other suitable structure within the device 1000) can include any suitable structure (e.g., shoulders, mounts, etc.) to apply a counterforce against the biasing members 1110. In such embodiments, the mounts can form an attachment point for the biasing members 1110 or the mounts can function as ground point for the biasing members. The mounts can be any suitable feature for locating, retaining, anchoring, or otherwise supporting feature for the biasing members.

In accordance with some embodiments, the biasing member 1110 produce a biasing range or distance (T1 minus T2). The biasing range is the unencumbered travel of the biasing mechanism in relationship to the stack up height T1 of the supported modules and/or components. Thus, the biasing range is the range of travel for the biasing mechanism that allows the support substrate to move to a position, when not restricted by supported modules or components, that is at a substrate distance T2 from the opposing interior housing surface. In some embodiments, the stack up height T1 is greater than the substrate distance T2. In this way, the biasing member 1110 are able to maintain a bias of the highest component in the module/component stack against the interior housing surface creating a component preload. In the example illustrated in FIG. 1, the detection surface 1821 is preloaded against the interior surface of the upper housing forming a contact region between the two.

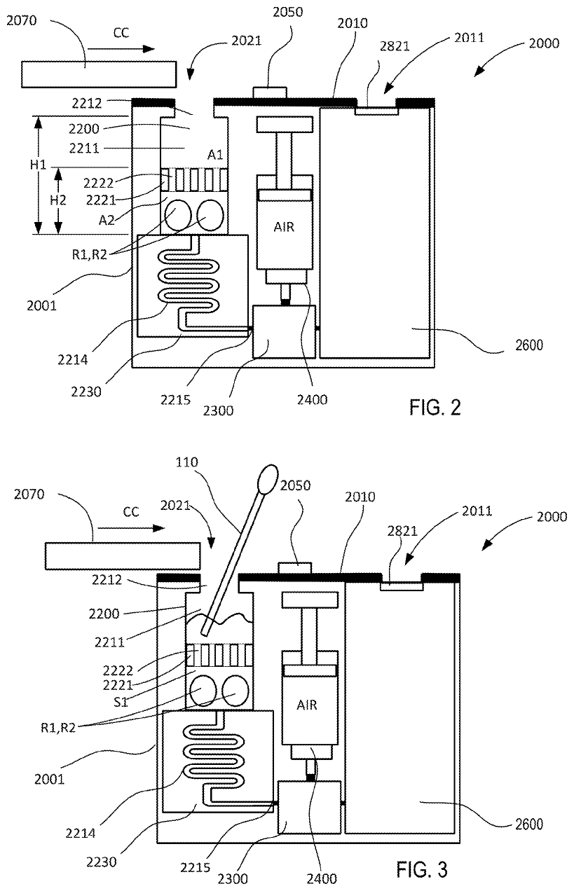

FIGS. 2 through 5 are schematic illustrations of a molecular diagnostic test device 2000 (also referred to as a “test device” or “device”), according to an embodiment. The test device 2000 is configured to manipulate biological sample to produce one or more output signals associated with a target cell, according to any of the methods described herein. In some embodiments, the test device 2000 can be an integrated device that is suitable for use within a point-of-care setting (e.g., doctor's office, pharmacy or the like), decentralized test facility, or at the user's home. Similarly stated, in some embodiments, the modules of the device, described below; are contained within a single housing such that the test device can be fully operated without any additional instrument, docking station, or the like. Further, in some embodiments, the device 2000 can have a size, shape and/or weight such that the device 2000 can be carried, held, used and/or manipulated in a user's hands (i.e., it can be a “handheld” device). In some embodiments, the test device 2000 can be a self-contained, single-use device.

The test device 2000 is configured to manipulate biological sample to produce one or more output signals associated with a target cell, according to the various systems and the methods described herein. In some embodiments, the test device 2000 can be an integrated device that is suitable for use within a point-of-care setting (e.g., doctor's office, pharmacy or the like), decentralized test facility, or at the user's home. Similarly stated, in some embodiments, the modules of the device, described below, are contained within a single housing such that the test device can be fully operated without any additional instrument, docking station, or the like. Further, in some embodiments, the device 2000 can have a size, shape and/or weight such that the device 2000 can be carried, held, used and/or manipulated in a user's hands (i.e., it can be a “handheld” device). In some embodiments, the test device 2000 can be a self-contained, single-use device.

In some embodiments, the device 2000 (and any of the devices shown and described herein) can be a CLIA-waived device and/or can operate in accordance with methods that are CLIA waived. Similarly stated, in some embodiments, the device 2000 (and any of the other devices shown and described herein) is configured to be operated in a sufficiently simple manner and can produce results with sufficient accuracy to pose a limited likelihood of misuse and/or to pose a limited risk of harm if used improperly. In some embodiments, the device 2000 (and any of the other devices shown and described herein), can be operated by a user with minimal (or no) scientific training, in accordance with methods that require little judgment of the user, and/or in which certain operational steps are easily and/or automatically controlled. In some embodiments, the molecular diagnostic test device 2000 can be configured for long term storage in a manner that poses a limited likelihood of misuse (spoilage of the reagent(s), expiration of the reagents(s), leakage of the reagent(s), or the like). In some embodiments, the molecular diagnostic test device 2000 is configured to be stored for up to about 36 months, up to about 32 months, up to about 26 months, up to about 24 months, up to about 20 months, up to about 18 months, up to 12 months, up to 6 months, or any values there between.

The test device 2000 includes a housing 2001, an actuator 2050, a sample preparation module 2200, a reaction module 2600, and a detection surface 2821. In some embodiments, the test device 2000 can include any other components or modules described herein, such as, for example, an amplification module, a detection module, a reagent module that contains on-board reagents (e.g., the reagent module 3700), a valve (e.g., to control flow of reagents and/or sample, such as the valve 2300), or a fluid transfer module (e.g., the fluid transfer module 2400). The housing 2001 can be any structure within which the sample preparation module 2200 or other components are contained (or partially contained) to form an integrated device for sample preparation and/or molecular testing. The housing 2001 can be a monolithically constructed housing or can include multiple separately constructed members that are later joined together to form the housing 2001 or be contained therein.

As shown in FIGS. 2-5, the housing 2001 includes an external wall 2010 that defines one or more external features. For example, the housing 2001 defines an housing opening 2021 through which a biological sample S1 can be conveyed into the sample preparation module 2200. The housing 2001 also defines a viewing area 2011 for seeing a visual display of test results or operation. The viewing area can include an opening through the external wall 2010 of the housing 2001.

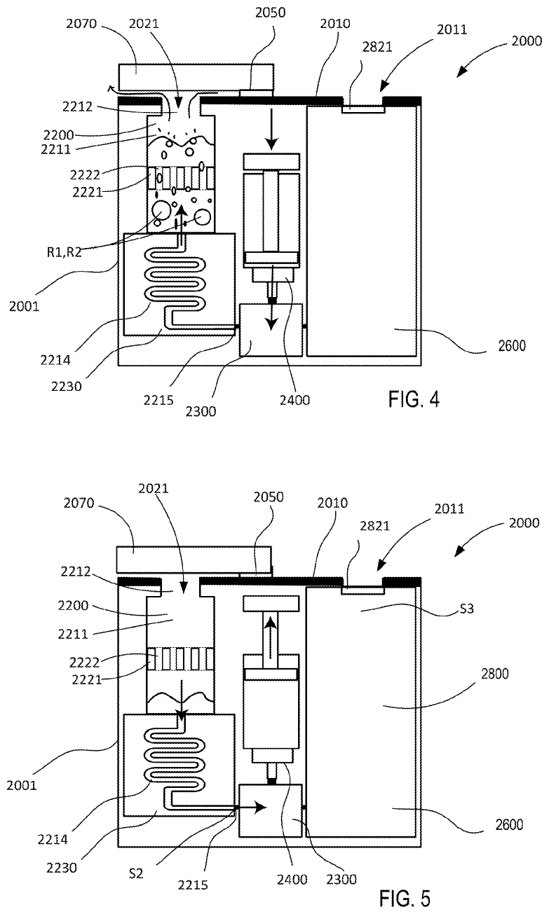

The sample preparation module 2200 is configured to manipulate the biological sample S1 for further diagnostic testing. For example, in some embodiments, the sample preparation module 2200 can extract target molecules (e.g., nucleic acid) from the biological sample S1 and can produce an input solution that is conveyed into the reaction module 2600. Referring to FIGS. 2 and 3, the sample preparation module 2200 defines a sample input opening 2212 and a sample input reservoir 2211 that receives a biological sample S1. The sample input opening 2212 is aligned with the housing opening 2021 and is an opening through which the sample input reservoir 2211 can be accessed. Thus, when the lid 2070 is in the opened position, the biological sample S1 can be conveyed into the input reservoir 2211 via the housing opening 2021 and the sample input opening 2212. In some embodiments, the biological sample S1 can be conveyed into the device by a sample transfer device 110. The sample transfer device 110 can be any suitable device, such as a pipette or other mechanism configured can be used to aspirate or withdraw the sample S1 from a sample cup, container or the like, and then deliver a desired amount of the sample via the opening 2021.

The sample preparation module 2200 can include any components as described herein to manipulate the biological sample S1 for further diagnostic testing and/or to produce a solution for detection of a target molecule (e.g., nucleic acid). The input reservoir 2211 is a volume within which the biological sample S1 can be mixed with reagents and also heated. Thus, in some embodiments, the sample preparation module 2200 can include one or more heaters, one or more chambers within which the biological sample S1 can be manipulated, one or more mixing reservoirs, and/or certain on-board reagents (e.g., a lysing buffer, an RT enzyme, a control substance, or the like). For example, in some embodiments the biological sample S1 can be collected in the input reservoir 2211 and mixed with either or both of a control organism (identified as reagent R1) and a reverse transcriptase (identified as reagent R2). The control organism and the reverse transcriptase can each be lyophilized or otherwise in solid form. In some embodiments, the solid reagent R1, R2 includes a lyophilized pellet. The lyophilized pellet(s) can include one or more of a reducing agent, positive control organism, reverse transcriptase enzymes, or salts. Moreover, the reagents R1 and R2 can be secured within the input reservoir 2211, as described herein, to prevent the reagents R1 and R2 from inadvertently falling out of the device 2000, for example during storage, transportation, or use.

In some embodiments, the sample preparation module 2200 can function merely as a sample holding or mixing reservoir. For example, in some embodiments, the sample preparation module 2200 can contain the desired amplification reagents to facilitate a desired amplification according to any of the methods described herein. In other embodiments, the sample preparation module 2200 is configured to extract nucleic acid molecules from the biological sample S1 and can produce an input solution S2 (see FIG. 2) that is conveyed into the reaction module 2600. For example, as shown, the input reservoir 2211 is in fluid communication with a serpentine flow channel 2214. In this manner, the biological sample that is mixed with the solid reagent can flow from input reservoir 2211 through the serpentine flow channel 2214, where the sample can be heated to perform lysing, a reverse transcription (also referred to herein as “RT”) reaction and/or an enzyme inactivation operation.

It will be appreciated that in different embodiments, the reverse transcription reaction can be performed at different points in the system. For example, in some embodiments, the reverse transcription reaction can be performed in the mixing reservoir. In this embodiment, the sample S1 is exposed to the high-heat lysis, then mixed with RT-PCR reagents at the same time in the mixing reservoir. Residual heat of the liquid coming out of the lysis process allows for temperatures that promote the reverse transcription within the mixing reservoir.

In accordance with some embodiments, the sample preparation module 2200 includes a retention screen 2221. The retention screen 2221 is positioned to separate the input reservoir 2211 into a first portion A1 and a second portion A2. The housing opening 2021 and the sample input opening 2212 extend into the first portion A1. The second portion A2 is separated from the sample input 2021 by the retention screen 2221, and contains a solid reagent (e.g., R1, R2). In this manner, the retention screen 2221 limits the movement of the solid reagent within (or out of) the input reservoir 2211. In particular, the solid reagent is limited from exiting the housing opening 2021 and/or the sample input opening 2212 by the retention screen 2221. The sample preparation module 2200 is configured to receive a biological sample and to mix the biological sample with the solid reagent R1, R2 to form an input solution S2 containing a target molecule.

In accordance with some embodiments, the retention screen 2221 defines one or more apertures 2222. The one or more apertures 2222 are sized to allow the biological sample S1 to flow through the retention screen 2221 from the first portion A1 of the input reservoir 2211 to the second portion A2 of the input reservoir 2211. In one example, the apertures are large enough to allow the biological sample S1 to flow through the retention screen 2221 from the first portion A1 into the second portion A2. Additionally or alternatively, the apertures are large enough to allow a mixing fluid (e.g., air and their bubbles in the biological sample as discussed below) to flow from the second portion A2 into the first portion A1. The one or more apertures 2222 are also sized to limit the ability of the solid reagent (e.g., R1, R2) within the second portion A2 of the input reservoir 2211 from exiting the input reservoir 2211 via the housing opening 2021. Specifically, the apertures are small enough to retain the solid reagent (e.g., R1, R2) within the second portion A2. For example, the aperture area is less than the solid reagent cross section.

In accordance with some embodiments, the input reservoir 2211 defines a volume that is suitable for containing the biological sample and mixing the biological sample with the solid reagent R1, R2. As indicated above, the retention screen 2221 is positioned within the input reservoir 2211 to separate the two sections of the input reservoir (i.e., portion A1 and portion A2). Each portion (i.e., portion A1 and portion A2) are a portion of the total volume of the input reservoir 2211. In some embodiments, the volume of the second portion A2 of the input reservoir 2211 is greater than half of the total volume. In other embodiments, the volume of the first portion A1 of the input reservoir 2211 is greater than half of the total volume. In yet other embodiments, the volume of the first portion A1 is about the same as the volume of the second portion A2. Thus, the retention screen 2221 can be positioned within the input reservoir 2211 at a position to maintain a desired volume ratio between the volume of the first portion A1, the volume of the second portion A2, and/or the total volume. Specifically, the retention screen 2221 can be positioned at any suitable height within the input reservoir 2211. In accordance with some embodiments, the input reservoir 2211 defines an interior height H1. The retention screen 2221 is positioned within the input reservoir 2211 at a height H2. In some examples, the ratio of the screen height H2 to the interior height H1 is between 0.3 and 0.6. In some examples, the ratio of the screen height H2 to the interior height H1 is about 0.5. In some examples, the second portion A2 is sufficiently small to limit the movement of the solid reagents such that they can be mixed with the biological sample with consistency. Thus, the ratio of H2 to H1 is one the allows the solid reagent (e.g., R1, R2) and the biological sample to suitably mix such that the resulting solution has a generally consistent concentration as it is pulled out of the input reservoir 2211.

In some embodiments, the mixing of the solid reagent or reagents are specifically controlled to obtain a generally consistent concentration of the reagent or reagents in the output solution from the input reservoir. For example, in some instances in which two solid reagents are used, one reagent might mix quickly compared to the other reagent. As a result, the early quantities of solution pulled from the input reservoir might have a disproportionate amount of the quickly dissolving reagent and the later quantities of the solution might have a disproportionately large amount of the slower dissolving reagent. Providing conditions for a controlled mixture, allows for a consistent delivery of the targeted amount of each of the reagents mixed with the biological sample.

In accordance with some embodiments, the mixing in the input reservoir 2211 between the biological sample and the solid reagents can be passive. For example, a passive mixing can occur by delivering the biological sample (e.g., via pipette) into the input reservoir 2211. In embodiments, having the retention screen 2221, the retention screen 2221 can diffuse the biological sample as it is added into the input reservoir 2211 allowing slow controlled contact with the solid reagents (e.g., R1, R2). Further passive mixing can occur by allowing the biological sample to reside in the input reservoir 2211 for a suitable time for the solid reagents to start to breakdown and mix with the biological sample. Mixing can be further controlled by orienting multiple solid reagents (e.g., R1, R2) in specific patterns within the input reservoir 2211. For example, the solid reagents can be placed on the same general plane (e.g., a horizontal plane relative to the surface upon which the device is placed) adjacent to one another. The reagents can be stacked vertically with respect to one another. The reagents can be positioned such that the biological sample contacts each of the reagents at about the same time. The reagents can be positioned relative to the outlet of the input reservoir (e.g., at substantially equal distance to the outlet) such that the outlet draws the solution around each of the reagents at similar rates providing similar concentrations. The reagents can be positioned relative to the outlet of the input reservoir such that one reagent is closer to the outlet than the other. This can cause a higher spike in the concentration of one reagent as compared to the other. Conversely, if the closer reagent breaks down slower in the solution, this orientation can balance the delivery concentrations of the reagents such that they are generally similar.

In some embodiments, the mixing in the input reservoir 2211 between the biological sample and the solid reagents can be active mixing. Active mixing can be provided in addition to or as an alternative to any one or more of the passive mixing protocols discussed above. Active mixing of the solution in the input reservoir can include any system that adds energy to the input reservoir causing the mixing. This can include forcing a fluid through the reservoir, vibrating the reservoir, using a device to stir the reservoir or other suitable systems. In one example, active mixing can include driving a fluid into the input reservoir 2211. The fluid can be any suitable fluid that would cause the biological sample and the solid reagents to mix.

In one example, the fluid includes gaseous fluids such as air suitable for bubbling through the input reservoir. While using forced air through the input reservoir for active mixing is used and described in more detail throughout, it is merely one example. Other examples discussed herein can also be utilized. For example, additionally or alternatively the fluid could be the biological sample itself. In this example, the drive module 2400 could push and pull the biological sample into and out of the input reservoir multiple times in succession to further mix the biological sample and the solid reagents.

Additionally, the device 2000 includes a set of fluid interconnects 2215 that allow for fluidic coupling of the sample preparation module 2200 to the fluid transfer valve 2300 and other components within the device 2000 (e.g., fluidic drive module 2400 and reaction module 2600). In this manner, the biological sample that is mixed with the solid reagent (also referred to as a reverse transcription solution) can flow from input reservoir 2211 through the serpentine flow channel 2214 and into the reaction module 2600. Furthermore, a pressure gradient (positive or negative) is applicable through the fluid transfer valve 2300 and serpentine flow channel 2214 into the input reservoir 2211 (e.g., via the fluidic drive module 2400). In this way, fluid can flow into the input reservoir 2211 via the serpentine flow channel 2214 and/or the mixed biological sample solution (e.g., the reverse transcription solution) can flow from the input reservoir 2211 (first volume) through the serpentine flow channel 2214. After the serpentine flow channel 2214, the prepared sample is then conveyed to the reaction module 2600.

In some embodiments, the fluid source (e.g., air for mixing) injected into the input reservoir 2211 from the flow channel 2214 is from the fluidic drive (or transfer) module 2400. In other embodiments, the fluid is from a dedicated source separate from the fluidic drive module 2400, such as separate reservoir (e.g., a compressed gas container). In some embodiments, the fluidic drive (or transfer) module 2400 is a pump or series of pumps configured to produce a pressure differential and/or flow of the solutions within the diagnostic test device 2000. Similarly stated, the fluid transfer module 2400 is configured to generate fluid pressure, fluid flow and/or otherwise convey the biological sample and the reagents through the various modules of the device 2000. In some embodiments, the fluid transfer module 2400 is configured to contact and/or receive the sample flow therein. As shown in FIGS. 2-5, the fluid transfer module 2400 can be a piston pump that is coupled to one or more of the sample preparation module 2200, the valve 2300, and/or the reaction module 2600. The fluid drive module 2400 can be driven by and/or controlled by the electronic control module. For example, in some embodiments, the fluid drive module 2400 can include a DC motor, the position of which can be controlled using rotary encoders (not shown). In other embodiments, a processor of the electronic control module can include code to and/or be configured to implement a closed loop method of tracking motor position by monitoring the current draw of motor, as described in International Patent Publication No. WO2016/109691, entitled “Devices and Methods for Molecular Diagnostic Testing.”

The valve assembly 2300 can be moved between various different configurations to allow flow of fluids within the device. In some embodiments, the valve assembly 2300 can direct a stored air supply in the fluid transfer module 2400 into the input reservoir 2211 to cause bubbling in the input reservoir 2211 to mix the biological sample and the solid reagent. The fluid transfer module 2400 can then be operated in the opposite direction to pull the mixed biological sample out of the input reservoir 2211 and into the serpentine flow channel 2214.

After completion of the sample preparation process, the valve assembly 2300 can be further moved into the second configuration (not shown). When the valve is in the second configuration, the mixed solution (e.g., post RT-PCR) can be conveyed into the reaction module 2600. The timing of the valve actuation and the power supplied to the fluidic drive module 2400 (e.g., the pump) can be controlled by the electronic control module (not shown in FIGS. 2-5) to maintain the flow rate through the amplification module 2600 within a range that the desired performance for the amplification can be achieved. Moreover, with the valve assembly 2300 in the second configuration, continued actuation of the fluidic drive module 2400 will convey the amplified solution into and through the reaction module 2600.

The reaction module 2600 defines an internal volume (e.g., a reaction chamber or reaction volume) that receives the input solution from the sample preparation module 2200 and amplifies a target molecule therein to produce an output containing a target amplicon. The reaction volume can be formed from any suitable structure that defines a volume or a series of volumes within which the input solution S2 can flow and/or be reacted to produce a solution for subsequent detection. Thus, the reaction module 2600 can function as an amplification module, a lysis module, or any other module within which a reaction can occur to facilitate detection of the target polynucleotide sequence. In some embodiments, the reaction module 2600 can amplify the target nucleic acid molecules therein to produce an output detection solution that contains a target amplicon (or multiple target amplicons) to be detected. For example, in some embodiments, the reaction module 2600 (or any of the reaction modules or amplification modules described herein) can be similar to the amplification modules shown and described in U.S. Patent Publication No. 2017/0304829, entitled “Printed Circuit Board Heater for an Amplification Module,” which is incorporated herein by reference in its entirety. In other embodiments, the amplification module 2600 (or any of the amplification modules described herein) can be similar to the amplification modules shown and described in International Patent Publication No. WO2016/109691, entitled “Devices and Methods for Molecular Diagnostic Testing.” Moreover, in accordance with some embodiments, the reaction module can include one or more components of an amplification modules and/or one or more components of a detection module.

Although the amplification modules described herein are generally described as performing a thermal cycling operation on the input solution S2, in other embodiments, the reaction module 2600 (and any of the amplification modules described herein) can perform any suitable thermal reaction to amplify nucleic acids within the solution. In some embodiments, the reaction module 2600 (and any of the amplification modules described herein) can perform any suitable type of isothermal amplification process, including, for example, Loop Mediated Isothermal Amplification (LAMP), Nucleic Acid Sequence Based Amplification (NASBA), which can be useful to detect target RNA molecules, Strand Displacement Amplification (SDA), Multiple Displacement Amplification (MDA), Ramification Amplification Method (RAM), or any other type of isothermal process

In accordance with some embodiments, the reaction module 2600 is configured to react the biological sample (identified as the processed solution S3 below) with one or more reagents to cause production of one or more assay signals to indicate presence of the target polynucleotide sequence. Although the biological sample is identified as a portion (i.e., S3) of the initial biological sample (i.e., S1) that has been processed, reacted or prepared within the sample preparation module 2200 and the reaction module 2600, in other embodiments, the portion of the biological sample that is further reacted within the reaction module 2600 can be any suitable portion of the initial biological sample S1. As described herein, the presence of the target polynucleotide sequence can indicate the presence of a target organism, whether the target organism is susceptible to a course of treatment, whether the target organism is resistant to a course of treatment, or other characteristics of the target organism. The reaction module 2600 can define a detection volume within which the biological sample and one or more reagents (see reagent R discussed below) can be reacted. The reacting can be performed by combining (e.g., mixing) the reagent R and the biological sample S3 within the reaction module 2600, by introducing each of the reagent R and the biological sample S3 into the reaction module 2600 (either at the same time or in a sequential manner), by conveying the biological sample S3 into the reaction module 2600, within which the reagent R has been stored for use, or any other suitable method for producing the desired reaction. In some embodiments, the reaction module 2600 can include one or more detection surfaces to which one or more probes are attached. As described herein, such probes can be designed to permit annealing or hybridization of a target amplicon with sufficient specificity to permit detection of the presence (or absence) of a target amplicon indicating the presence of the target polynucleotide sequence. In other embodiments, the detection module can include one or more detection chambers in which different reagents or probes can be combined or reacted with the biological sample to produce a series of assay signals.

The detection surface 2821 is a surface from which the target amplicon is detectable by producing a signal. Specifically, as described herein, the output solution from (or within) the amplification module 2600 can be reacted with one or more reagents to produce a signal (or output) to indicate presence or absence of a target organism in the biological sample S1. In some embodiments, the detection surface 2821 can retain the target amplicon produced during amplification within a single region to produce a signal. In some embodiments, the detection surface 2821 can include a series of capture probes to which the target amplicon can be bound when the output solution is in contact with the detection surface 2821 (e.g., according to the enzyme-linked reaction process shown and described with reference to FIG. 7A). The capture probes can be any suitable probe of the types described herein formulated to capture or bind to the target amplicon.

In some embodiments, the detection surface 2821 is within the reaction module 2600. For example, in some embodiments, a portion of a wall that defines the reaction volume can also include the detection surface 2821. In this manner, the amplification reaction and reaction to produce the signal can occur within the same module (i.e., the reaction module 2600). In other embodiments, the detection surface 2821 can be spaced apart from (or outside of) the reaction module 2600. For example, in some embodiments the device 2000 can include a separate detection module (not shown) that defines a detection channel and includes the detection surface 2821 within the detection channel. The detection channel is in (or can be placed in) fluid communication with the reaction module 2600. In this manner, the output solution containing the target amplicon can be conveyed into the detection channel and across the detection surface 2821.

The housing 2001 includes a lid 2050 that is movably coupled thereto. As shown by the arrow CC, the lid 2050 is configured to move relative to the housing 2001 from a first (or opened) position to a second (or closed) position (see FIGS. 2-5). When the lid 2050 is in the opened position, the housing opening 2021 and the sample input opening 2212 are exposed, thereby allowing the biological sample S1 to be conveyed into the sample preparation module 2200. After the biological sample S1 is loaded, the user can close the lid 2050 (i.e., can move the lid to its second position). The lid 2050 covers the housing opening 2021 and the sample input opening 2212 when the lid 2050 is in the closed position. In some examples, the lid 1050 is spaced apart from housing opening 2021 and/or the sample input opening 2212 to define an air gap to the input reservoir. Said another way, in some embodiments, the lid 2050 does not seal closed the housing opening 2021 and/or the sample input opening 2212. This air gap can limit pressurization of the input reservoir when the device is active, thereby allowing flow of air into the sample input reservoir 2211 to enhance mixing. In other examples, the lid 2050 includes a seal, gasket, or other material to fluidically isolate the sample input reservoir 2211 when the lid 2050 is in the second lid position.

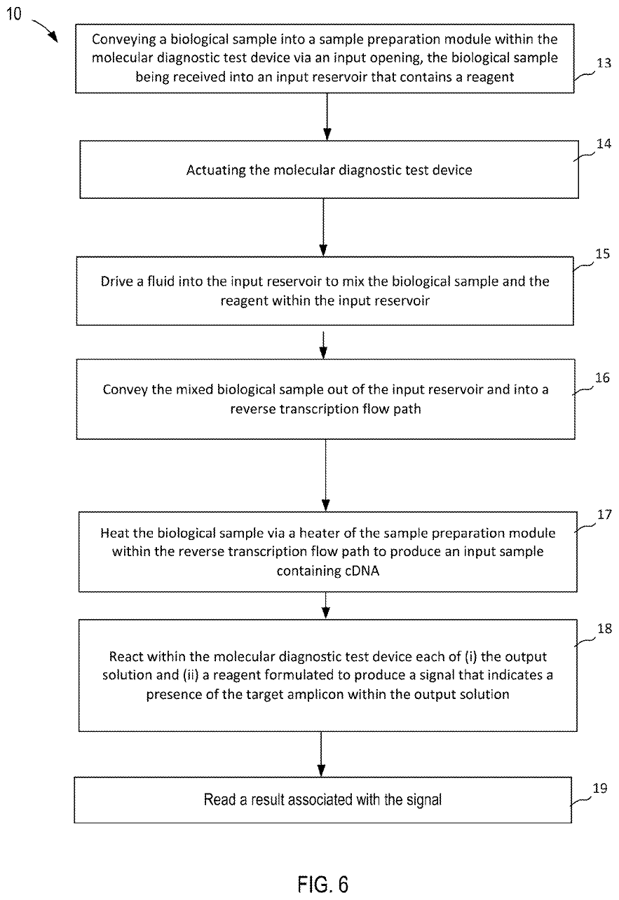

The molecular diagnostic test device 2000 (and any of the molecular diagnostic test devices described herein) can perform any of the processes described herein. For example, FIG. 6 is a flow chart of a method 10 of detecting a target molecule (e.g., a nucleic acid), according to an embodiment. Although the method 10 is described as being performed on the device 2000, in other embodiments, the method 10 can be performed on any suitable device, such as the device 3000 described below. A biological sample is conveyed into a sample preparation module within the molecular diagnostic test device via an input opening, at 13. The biological sample is received into an input reservoir that contains a reagent. Referring to FIGS. 3, in some embodiments, the biological sample S1 can be conveyed into the device by a sample transfer device 110. The sample transfer device 110 can be any suitable device, such as a pipette or other mechanism configured can be used to aspirate or withdraw the sample S1 from a sample cup, container or the like, and then deliver a desired amount of the sample via the opening 2021. The biological sample S1 can be any suitable sample, such as, for example, blood, urine, male urethral specimens, vaginal specimens, cervical swab specimens, nasal swab specimens, throat swab specimens, rectal swab specimens, or any other biological samples described herein. Thus, in some embodiments, the biological sample S1 can be a “raw” (or unprocessed) sample.

The molecular diagnostic test device is then actuated, at 14, which causes the molecular diagnostic test device to perform a series of operations. In one example, the molecular diagnostic test device is actuated via the actuator 2050 in FIG. 4. Although the actuator 2050 is shown as a push-button style actuator, in other examples, the operation 14 can be performed by any suitable mechanism. For example, in some embodiments, the device can be actuated by sliding the lid 2070 closed to actuate the device. In other embodiments, the device can include an actuator that is user contacted to begin device operation. In some embodiments, the actuator can a device that is removed from (e.g., peeled from) the device to begin device operation.

Actuation of the device causes the device to perform a series of operations, including driving a fluid into the input reservoir 2211 to mix the biological sample S1 and the reagent (e.g., the reagents R1, R2) within the input reservoir 2211, at 15. This mixing includes forcing the fluid under pressure up through the input reservoir 2211 while the input reservoir 2211 contains the biological sample S1 and the reagent R1, R2. In one example, as discussed above, the fluid is air that is forced through the input reservoir 2211 causing bubbles. The bubbles cause turbulence withing the mixture of biological sample S1 and the reagent R1, R2 further mixing the biological sample S1 and the reagent R1, R2. While shown in FIG. 3 as air, other suitable fluids can be utilized as well. In other examples, a liquid suitable to cause turbulence can also or alternatively be used. The biological sample S1 is loaded in a first direction within a flow path into the input reservoir 2211 and the fluid (e.g., air) is injected into the input reservoir from a second direction along the flow path. The fluid is injected into the input reservoir 2211 for a suitable time to mix the biological sample and the solid reagent R1, R2 such that when the solution is pulled from the input reservoir the reagents are mixed into the biological sample in concentrations that remain generally consistent through the process. In one example, the air pushed through the input reservoir 2211 between 10-30 μL/s. In one example, the air is pushed through the input reservoir 2211 at between 15-25 μL/s. In one example, the air is pushed through the input reservoir at about 20 μL/s.

In some embodiments, as discussed above, the biological sample S1 can be pulled and pushed back and forth into the input reservoir 2211. In examples of this embodiment, the biological sample S1 can be pushed/pulled back and forth rates and for lengths of time suitable to achieve the generally consistent concentration. In one example, 40-80% of the volume of liquid in the input reservoir 2211 can be toggled back and forth. In one example, 50-60% of the volume of liquid in the input reservoir 2211 can be toggled back and forth. In one example, about 60% of the volume of liquid in the input reservoir 2211 can be toggled back and forth. In one example, the liquid in the input reservoir 2211 can be toggled back and forth at a rate of between 5-30) μL/s. In one example, the liquid in the input reservoir 2211 can be toggled back and forth at a rate of between 10-20 μL/s. In one example, the liquid in the input reservoir 2211 can be toggled back and forth at a rate of about 10 μL/s. In one example, the liquid in the input reservoir 2211 can be toggled back and forth at a rate of about 20 μL/s. In one example, the liquid is toggled back and forth at least 5 times. In one example, the liquid is toggled back and forth at least 10 times. In one example, the liquid is toggled back and forth 10 times.

In some embodiments, the fluidic drive 2400 (see FIG. 3) is used to store the fluid supply used to create the turbulence in the input reservoir 2211 as shown in FIG. 4. The transfer of fluids is caused by the fluidic drive (or transfer) module 2400. The fluidic drive (or transfer) module 2400 can be a pump or series of pumps configured to produce a pressure differential and/or flow of the solutions within the diagnostic test device 2000. Similarly stated, the fluid transfer module 2400 is configured to generate fluid pressure, fluid flow and/or otherwise convey the biological sample and the reagents through the various modules of the device 2000. As shown in FIGS. 2-5, in some embodiments, the fluid transfer module 2400 can be a piston pump. Prior to activation, the piston pump is configured to store the turbulence fluid (e.g., air). Upon activation, the piston pump is actuated forcing the stored air out of the pump and though the input reservoir 2211 causing bubbles forming turbulence therein. The turbulence increases the interaction between the biological sample S1 and the reagent R1, R2 compared to a passive mixing process. In some embodiments, the input reservoir can include a bleed port or other mechanism to allow the air used for mixing to escape the input reservoir (e.g., to prevent over-pressurization). For example, in some embodiments the lid of the device is spaced apart from sample input opening 2021 to form an air gap between the lid 2070 and the input reservoir 2211. In response to the air driven into the input reservoir, the air gap allows some air to pass through the air gap and out of the input reservoir 2211 limiting the pressure therein.

After the mixing, the fluidic drive 2400 (see FIG. 5) is used to convey the mixed biological sample out of the input reservoir 2211 and further along the preparation module. For example, the mixed biological sample can be introduced into a reverse transcription flow path, at 16. As shown in FIG. 5 the piston pump is retracted thereby pulling the mixed biological sample out of the input reservoir 2211 and into a serpentine 2214 coupled with heater 2230. For example, as shown between FIGS. 4 and 5, the piston pump is driven in a first piston direction in response to actuation of the molecular diagnostic test device to drive air into the input reservoir 2211 along the reverse transcription flow path causing bubbles in the input reservoir and the piston is moved in a second piston direction to convey the mixed biological sample into the reverse transcription flow path.

The device 2000 heats the biological sample via a heater of the sample preparation module to produce an input solution S2, at 17. Referring to FIG. 5, the biological sample S1 can be heated by the heater 2230) and the resulting sample (i.e., the input solution S2) can be conveyed towards the reaction module 2600. Specifically, in some embodiments, the sample can be heated to perform a reverse transcriptase reaction, and the sample can containing cDNA. Although the device 2000 does not show any additional sample preparation, in other embodiments, the biological sample can be filtered, separated, eluted, subjected to an enzyme inactivation heating operation, or the like to produce a suitable input solution S2. In other embodiments, however, the method need not include any filtering or other separation techniques.

The input solution S2 is then conveyed to the reaction module 2600 within the molecular diagnostic test device. Referring to FIG. 5, the reaction module 2600 defines a reaction volume, as described above. Accordingly, the input solution S2 is further processed within the reaction volume to amplify the nucleic acid within the input solution thereby producing an output solution containing a target amplicon, at 18. The input solution can be amplified by using any suitable technique (e.g., PCR, isothermal amplification, etc.), as described herein. For example, the input solution S2 can be heated within a reaction volume within the molecular diagnostic test device to amplify the cDNA thereby producing an output solution containing a target amplicon. After amplification, the device then reacts within a reaction module 2600 within the molecular diagnostic test device each of (i) the output solution and (ii) a reagent formulated to produce a signal are further processed to indicate a presence of the target amplicon within the output solution S3. As shown in FIG. 3, the reaction module 2600 includes a detection surface 2821 configured to capture the target amplicon to produce the output signal. The output signal can be any suitable signal. In some embodiments, the output signal can be a colorimetric signal that indicates the presence of bound amplicon: if the target pathogen, target amplicon and/or target organism is present, the color product is formed, and if the target pathogen, target amplicon and/or target organism is not present, the color product does not form.

The reagent R can be any suitable reagent of the types described herein and can be introduced into the reaction module 2600 by any suitable mechanism. For example, in some embodiments, the reagent can be a catalyst formulated to be bound to the target molecule. In other embodiments, the reagent can be formulated to produce the signal when catalyzed by another reagent already present in the reaction module 2600. In some embodiments, the reagent can be a precipitating substrate formulated to produce an insoluble colored particle when the reagent is contacted with a catalyzing agent. The reagent R can present in the detection module before the device is actuated or alternatively, the reagent R can be conveyed into the detection module as a result of the device actuation. For example, in some embodiments, the device can include an on-board reagent module (e.g., reagent module 3700), and when the device is actuated, the device can release the reagent into a manifold or “holding tank” for later use during the procedure. In some embodiments, the device can include a fluid transfer device or a pump, similar to the fluid transfer device 2400 described herein.

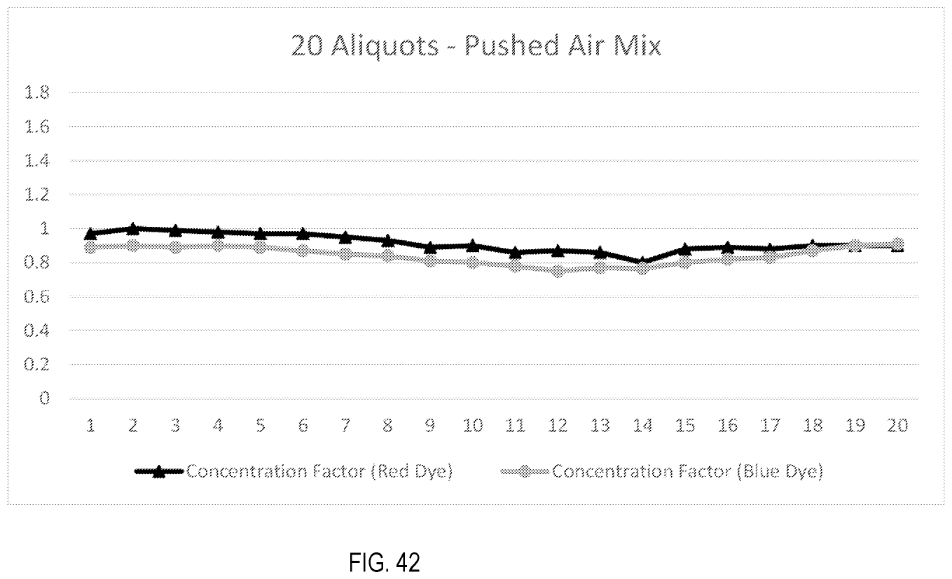

The method further includes reading a result associated with the signal, at 19. In some embodiments, the reading can include visually inspecting the device and the detection surface 2821 for a colorimetric signal. In other embodiments, the signal produced by the detection surface 2821 need not be visible to the naked eye. For example, in some embodiments, the reading can include using a secondary device, such a mobile computing device to scan or otherwise receive the signal (see e.g., FIG. 43). In vet other embodiments, the reading the result can include indirectly reading a secondary signal that conveys the results associated with (or describing) the primary output from the detection surface 2821. For example, in some embodiments, the device 2000 can include an electronic detection module (not shown in FIGS. 2-5) that analyzes the signal produced and, in turn, produces an output (e.g., an LED output, an audible output, a wireless signal output) to convey the test results to the user.

In some embodiments, the method 10 optionally includes discarding, after the reading, the molecular test device. In some embodiments, the amount of sample and reagents can be such that the device can be disposed of via standard, non-regulated waste procedures. In other embodiments, the discarding includes disposing of the used device via standard medical waste procedures. In yet other embodiments, the device can be discarded by returning it to a recycling facility where certain components of the device can be refurbished for possible re-use.

In some embodiments, the method 10 optionally includes storing the molecular diagnostic test device including any reagents sealed therein for at least six months before use.

Although the method 10 contemplates coupling the device to the power source as occurring before the biological sample is conveyed into the device, in other embodiments, any of the steps of the method 10 (or any of the methods described herein) can be performed in any order or can be performed concurrently. For example, in some embodiments, the biological sample S1 can be conveyed into the device first, the device can be actuated (via the actuator 2050), and then after actuation, the device can be plugged in to an outlet to provide A/C power to the device. The molecular diagnostic test device 2000 includes a control module having a switch and a processor that control transmission of power to the pump of the molecular diagnostic test device for driving air to the input reservoir and pulling the mixed biological sample out of the input reservoir.

FIG. 7A illustrates a portion of the operations and/or features associated with an enzymatic reaction, according to an embodiment, that can be conducted by or within any reaction modules (e.g., 1600, 2600, and 4600) and any detection module described herein (e.g., the detection module 3800). In some embodiments, the enzymatic reaction can be carried out to facilitate visual detection of a molecular diagnostic test result using the device 1000, 2000, 3000, and 4000, or any other devices or systems described herein. In other embodiments, the enzymatic reaction need not be performed to produce visual detection. For example, as described herein, in some embodiments, the methods that employ the illustrated enzymatic reaction can employ alternative methods to read a result associated with signal produced.

In some embodiments, the devices (including any of the various devices shown and described herein) can be configured for use in a decentralized test facility. Further, in some embodiments, the reaction shown in FIG. 7A can facilitate the devices (including any of the various devices shown and described herein) operating with sufficient simplicity and accuracy to be a CLIA-waived device. Similarly stated, in some embodiments, the reaction shown in FIG. 7A can provide the output signal OP1 in a manner that poses a limited likelihood of misuse and/or that poses a limited risk of harm if used improperly. In some embodiments, the reaction can be successfully completed within the device (or any other device described herein) upon actuation by a user with minimal (or no) scientific training, in accordance with methods that require little judgment of the user, and/or in which certain operational steps are easily and/or automatically controlled.

In some embodiments, the detection module 4800 is included as a part of the reaction module (e.g., 1600, 2600, 3600, 6600, etc.). In other embodiments, the reaction modules function without a specific detection module but include a detection surface 4821 within a read lane or flow channel. In embodiments having a detection module 4800 (which can also include device 3000), the detection module 4800 includes a detection surface 4821 within a read lane or flow channel. The detection surface 4821 is spotted and/or covalently bonded with a specific hybridizing probe 4870, such as an oligonucleotide. The hybridizing probe 4870) (also referred to as a capture probe) can be similar to any of the capture probes described herein. In some embodiments, the hybridizing probe 4870 is specific for a target organism, nucleic acid, and/or amplicon. The bonding of the hybridizing probe 4870 to the detection surface 4821 can be performed using any suitable procedure or mechanism. For example, in some embodiments, the hybridizing probe 4870 can be covalently bound to the detection surface 4821.

Reference S3 illustrates the biotinylated amplicon that is produced from the amplification step such as, for example, by an amplification module (or any other amplification modules or processes described herein such as those operating within a reaction module such as 1600, 2600, and 4600). The biotin can be incorporated within the amplification operation and/or within the amplification module in any suitable manner. As shown by the arrow XX, the output from the amplification module, including the biotinylated amplicon S3 is conveyed within the read lane and across the detection surface 4821. The hybridizing probe 4870 is formulated to hybridize to the target amplicon S3 that is present within the flow channel and/or in proximity to the detection surface 4821. The detection module 4800 and/or the detection surface 4821 is heated to incubate the biotinylated amplicon S3 in the read lane in the presence of the hybridizing probe 4870 for a few minutes allowing binding to occur. In this manner, the target amplicon S3 is captured and/or is affixed to the detection surface 4821, as shown. Although disclosed as being labeled with biotin, in other embodiments, the target molecules can be labeled in any suitable manner that will allow binding of the complex comprising a sample molecule binding moiety and an enzyme capable of facilitating a colorimetric reaction. For example, in some embodiments, the target molecules can be labeled with one or more of the following: streptavidin, fluorescein, Texas Red, digoxigenin, or Fucose.

In some embodiments, a first wash solution (not shown in FIG. 7A) can be conveyed across the detection surface 4821 and/or within the flow channel to remove unbound PCR products and/or any remaining solution. Such wash solution can be, for example, a multi-purpose reagent, and the first reagent R1. In other embodiments, however, no wash operation is conducted.

As shown by the arrow YY, a detection reagent R5 is conveyed within the read lane and across the detection surface 4821. The detection reagent R5 can be any of the detection reagents described herein. In some embodiments, the detection reagent R5 can be a horseradish peroxidase (HRP) enzyme (“enzyme”) with a streptavidin linker. In some embodiments, the streptavidin and the HRP are cross-linked to provide dual functionality. As shown, the detection reagent is bound to the captured amplicon S3. The detection module 4800 and/or the detection surface 4821 is heated to incubate the detection reagent R5 within the read lane in the presence of the biotinylated amplicon S3 for a few minutes to facilitate binding.

In some embodiments, a second wash solution (not shown in FIG. 7A) can be conveyed across the detection surface 4821 and/or within the flow channel to remove unbound detection reagent R5. Such wash solution can be, for example, a multi-purpose reagent, as described with reference to the first reagent R1. In other embodiments, however, no second wash operation is conducted.

As shown by the arrow ZZ, a detection reagent R6 is conveyed within the read lane and across the detection surface 4821. The detection reagent R6 can be any of the detection reagents described herein. The detection reagent R6 can be, for example, a substrate formulated to enhance, catalyze and/or promote the production of the signal OP1 when reacted with the detection reagent R5. Specifically, the substrate is formulated such that upon contact with the detection reagent R5 (the HRP/streptavidin) color molecules are produced. As such, a colorimetric output signal OP1 is developed where HRP attaches to the amplicon. The color of the output signal OP1 indicates the presence of bound amplicon: if the target pathogen, target amplicon and/or target organism is present, the color product is formed, and if the target pathogen, target amplicon and/or target organism is not present, the color product does not form.

In some embodiments the detection reagent R6 can be continuously flowed across the detection surface 4821 to ensure that the reaction producing the color molecules does not become limited by the availability of the detection reagent. Moreover, in some embodiments, the detection reagent R6 can be a precipitating substrate.

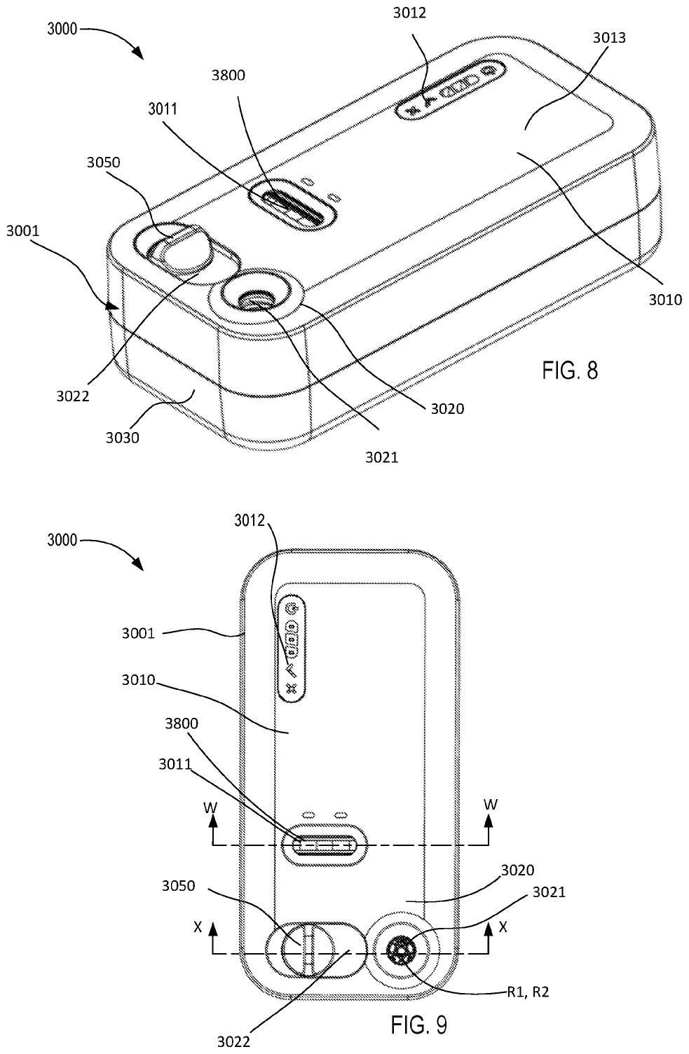

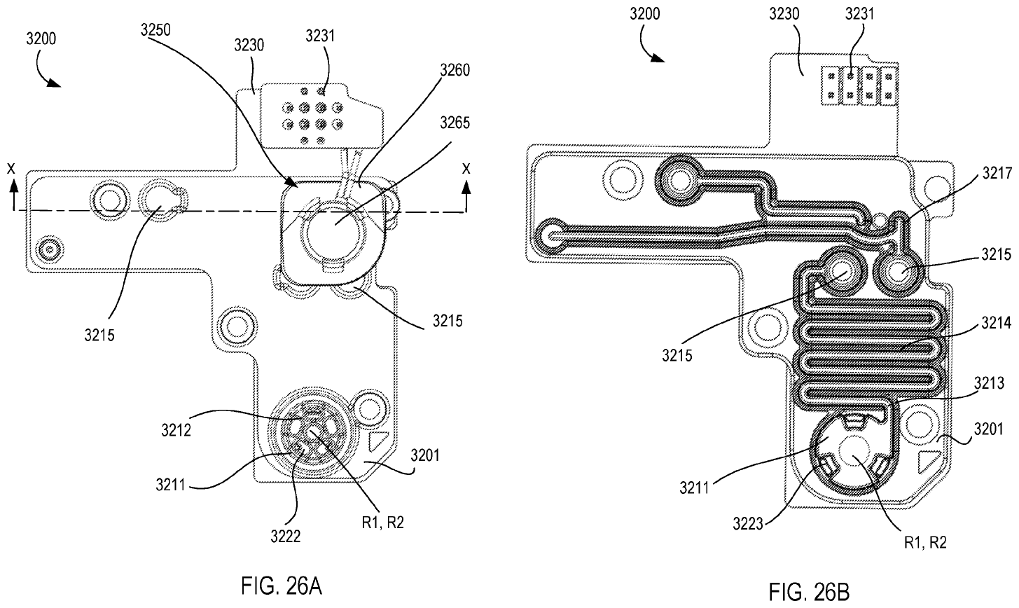

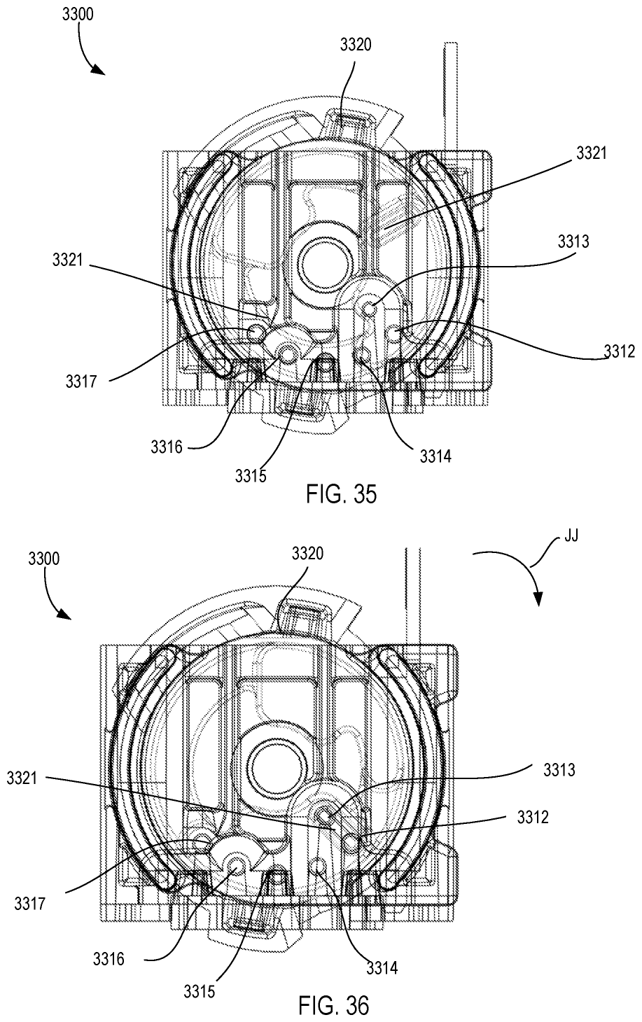

FIG. 7B is a schematic illustration of a molecular diagnostic test device 3000, according to an embodiment. The schematic illustration describes the components of the test device 3000 as shown in FIGS. 8-40. The test device 3000 is an integrated device (i.e., the modules are contained within a single housing) that is suitable for use within a point-of-care setting (e.g., doctor's office, pharmacy or the like), decentralized test facility (e.g., work sites or public venues), or at the user's home. In some embodiments, the device 3000 can have a size, shape and/or weight such that the device 3000 can be carried, held, used and/or manipulated in a user's hands (i.e., it can be a “handheld” device). A handheld device may have dimensions less than 15 cm×15 cm×15 cm, or less than 15 cm×15 cm×10 cm, or less than 12 cm×12 cm×6 cm. In other embodiments, the test device 3000 can be a self-contained, single-use device. Similarly stated, the test device 3000 is a stand-alone device that includes all necessary substances, mechanisms, and subassemblies to perform any of the molecular diagnostic tests described herein. As such, the device 3000 does not require any external instrument to manipulate the biological samples, and only requires a connection to a power source (e.g., a connection to an A/C power source, coupling to a battery, or the like) to complete the methods described herein. In some embodiments, the test device 3000 can be configured with lock-outs or other mechanisms to prevent re-use or attempts to re-use the device.

Further, in some embodiments, the device 3000 can be a CLIA-waived device and/or can operate in accordance with methods that are CLIA waived. Similarly stated, in some embodiments, the device 3000 (and any of the other devices shown and described herein) is configured to be operated in a sufficiently simple manner, and can produce results with sufficient accuracy to pose a limited likelihood of misuse and/or to pose a limited risk of harm if used improperly. In some embodiments, the device 3000 (and any of the other devices shown and described herein), can be operated by a user with minimal (or no) scientific training, in accordance with methods that require little judgment of the user, and/or in which certain operational steps are easily and/or automatically controlled. In some embodiments, the molecular diagnostic test device 3000 can be configured for long term storage in a manner that poses a limited likelihood of misuse (spoilage of the reagent(s), expiration of the reagents(s), leakage of the reagent(s), or the like). In some embodiments, the molecular diagnostic test device 3000 is configured to be stored for up to about 36 months, up to about 32 months, up to about 26 months, up to about 24 months, up to about 18 months, up to about 6 months, or any values there between.

The test device 3000 is configured to manipulate a biological sample S1 to produce one or more output signals associated with a target cell. Specifically, the device 3000 includes a sample preparation module 3200, a fluidic drive (or fluid transfer) module 3400, an amplification module 3600, a detection module 3800, a reagent module 3700, a valve 3300, and a control module (not shown). The test device and certain components therein can be similar to any of the molecular test devices shown and described herein or in U.S. Patent Publication No. US2019/0169677, entitled “Portable Molecular Diagnostic Device and Methods for Detection of Target Viruses.” Accordingly, a detailed description of certain modules (e.g., the fluidic drive module 3400) is not provided herein. A description of each of the modules is provided below.

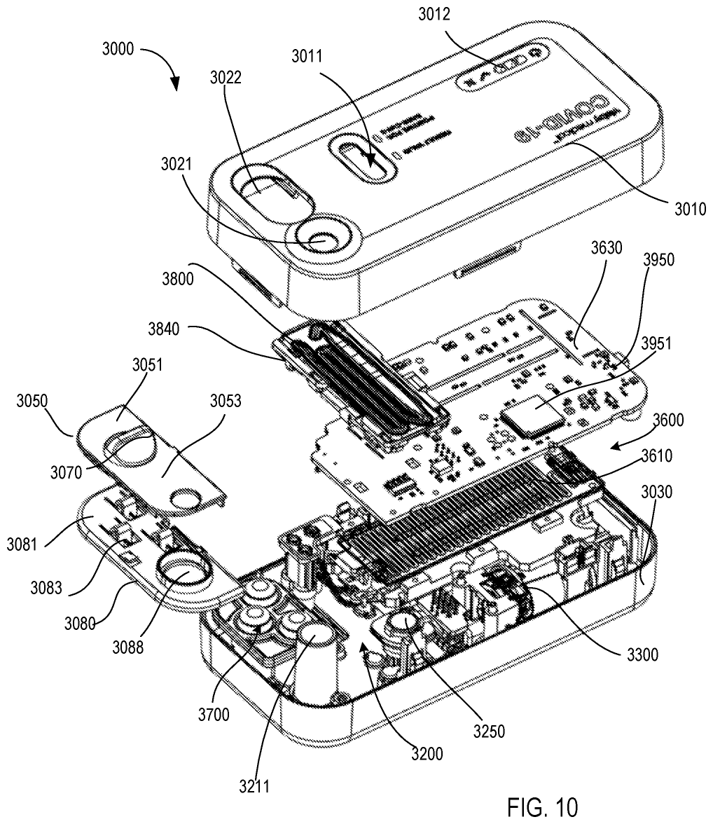

FIGS. 8-41C show various views of the molecular diagnostic test device 3000. The test device 3000 is configured to manipulate an input solution to produce one or more output signals associated with a target cell, according to any of the methods described herein. The diagnostic test device 3000 includes a housing 3001 (including a top housing or portion 3010 and a bottom portion 3030), within which the modules described herein are fully or partially contained. Similarly stated, the housing 3001 (including the top housing or portion 3010 and/or the bottom housing or portion 3030) at least partially surround and/or enclose the modules. FIGS. 10-13 are various views that show the sample preparation module 3200, the fluidic drive (or fluid transfer) module 3400, the amplification module 3600, the detection module 3800, the reagent module 3700, the fluid transfer valve 3300, and the electronic control module 3950 situated within the housing 3001. A description of the housing assembly 3001 is followed by a description of each module and/or subsystem.

The housing assembly 3001 includes a top housing 3010 (also referred to as the top wall), a bottom housing (also referred to as bottom wall) 3030, and a lid 3050 (which functions as a cover and an actuator). As shown, the top housing 3010 defines a detection opening 3011 (also referred to as a detection window or viewing area) and a series of status light openings 3012. The top housing 3010 also includes a sample input portion 3020 and a label 3013. The status light openings 3012 are aligned with one or more light output devices (e.g., LEDs) of the electronic control module 3950. In this manner, a light output produced by such status lights is visible through the status light openings 3012. Such light outputs can indicate, for example, whether the device 3000 is receiving power from the power source, whether an error has occurred (e.g., an error associated with insufficient sample volume or the like), and whether the test has been successfully completed.

The detection opening (or window) is aligned with the detection module 3800. In this manner, the signal produced by and/or on each detection surface of the detection module 3800 is visible through the detection opening 3011. In some embodiments, the top housing 3010 and/or the label 3013 is opaque (or semi-opaque), thereby “framing” or accentuating the detection opening. In some embodiments, for example, the top housing 3010 can include markings (e.g., thick lines, colors or the like) to highlight the detection opening 3011. For example, in some embodiments, the top housing 3010 can include indicia identifying the detection opening to a specific disease (e.g., SARS-CoV-2, Chlamydia trachomatis (CT), Neisseria gonorrhea (NG) and Trichomonas vaginalis (TV)) or control. Moreover, as described in detail herein, the detection module 3800 is biased against the top housing 3010 to minimize the distance between the detection module 3800 and the detection opening 3011. This biased arrangement minimizes shadows and optical aberrations and improves readability of the test results. In other embodiments, the top housing 3010 need not include a detection opening 3011. For example, in such embodiments, the signal produced by the detection module 3800 is not visible to the naked eye, but instead is read using another method. For example, in some embodiments, the reading can include using a secondary device, such a mobile computing device to scan or otherwise receive the signal OP1. In yet other embodiments, the reading the result can include indirectly reading a secondary signal that conveys the results associated with (or describing) the primary output from the detection module 3800.

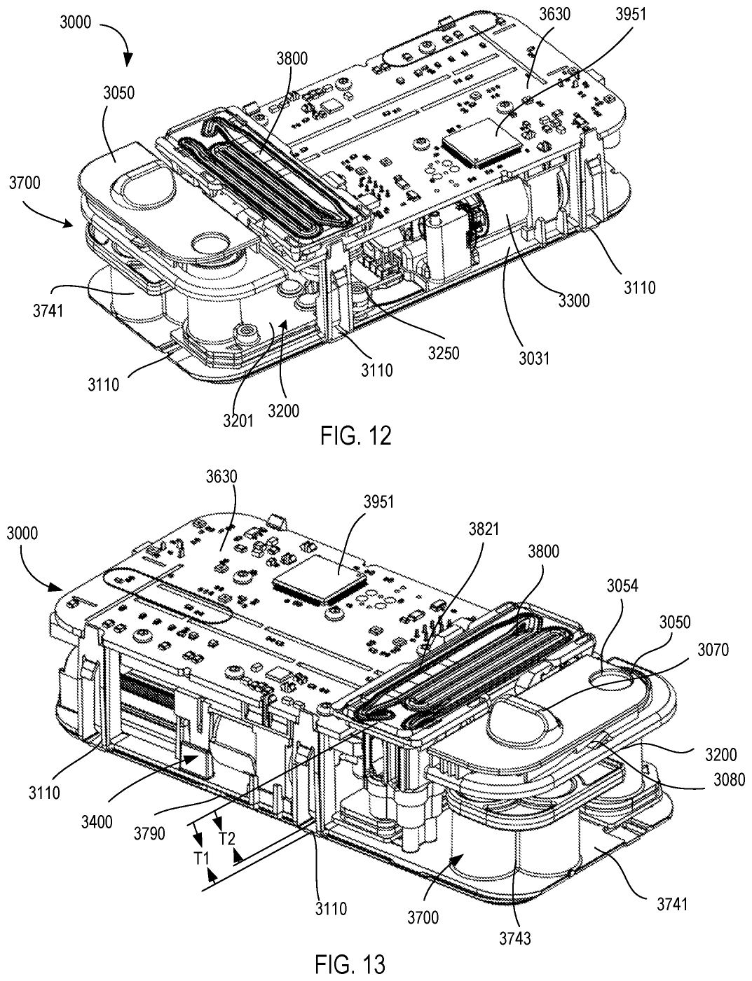

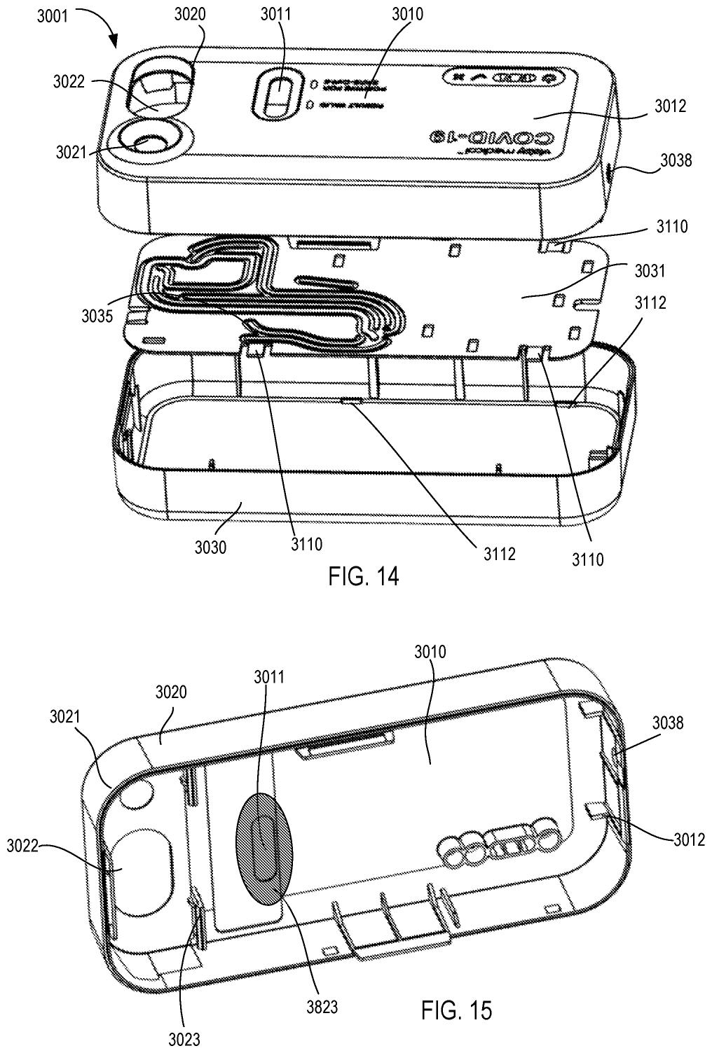

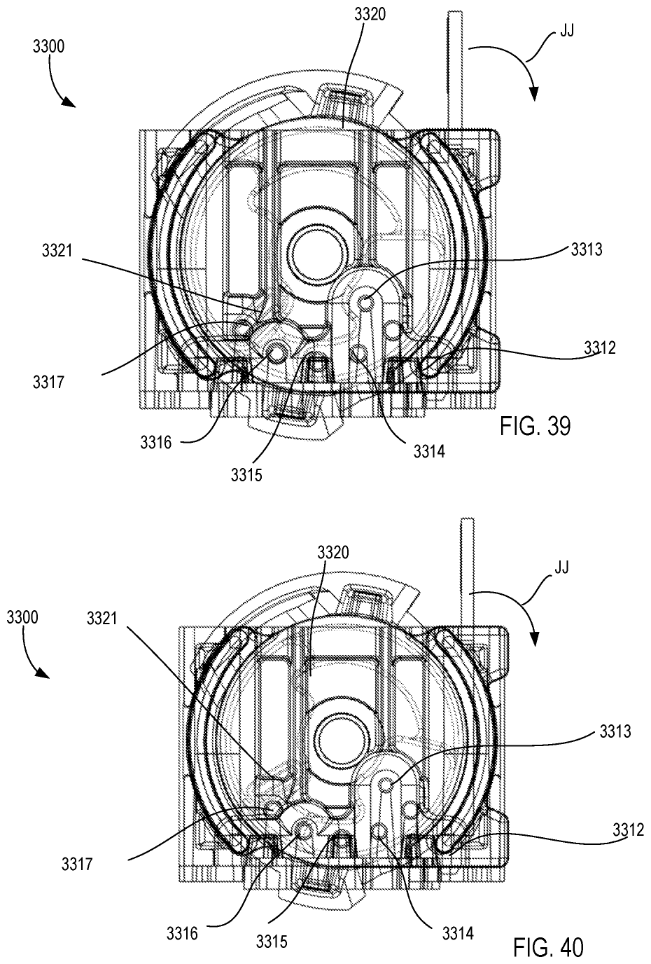

Referring to FIGS. 14 and 15, the sample input portion 3020 includes a set of guide rails 3023. The sample input portion 3020 also defines a sample input opening 3021 (also referred to as a housing input opening) and an actuator opening 3022. The sample input opening 3021 is aligned with the input opening 3212 (of the sample preparation module 3200) and provides an opening through which a biological sample S1 can be conveyed into the device 3000. Additionally, the sample input portion also allows the lid (or actuator) 3050 to be movably coupled to the top housing 3010. Specifically, as shown in FIGS. 8, 21, and 34, the lid 3050 is coupled to the top housing 3010 such that the handle 3070 of the actuator extends through the actuator opening 3022. The actuator opening 3022 is elongated to allow for sliding movement of the lid 3050 relative to the top housing 3010, as described herein. As shown in FIG. 14, the top housing 3010 defines an opening 3038 that is aligned with a power input port of the electronic control module 3950. In use, an end of a power cord can be coupled to the electronic control module 3950 via the opening 3038 (see e.g., the coupling of the power cord 3905 in FIG. 41C).

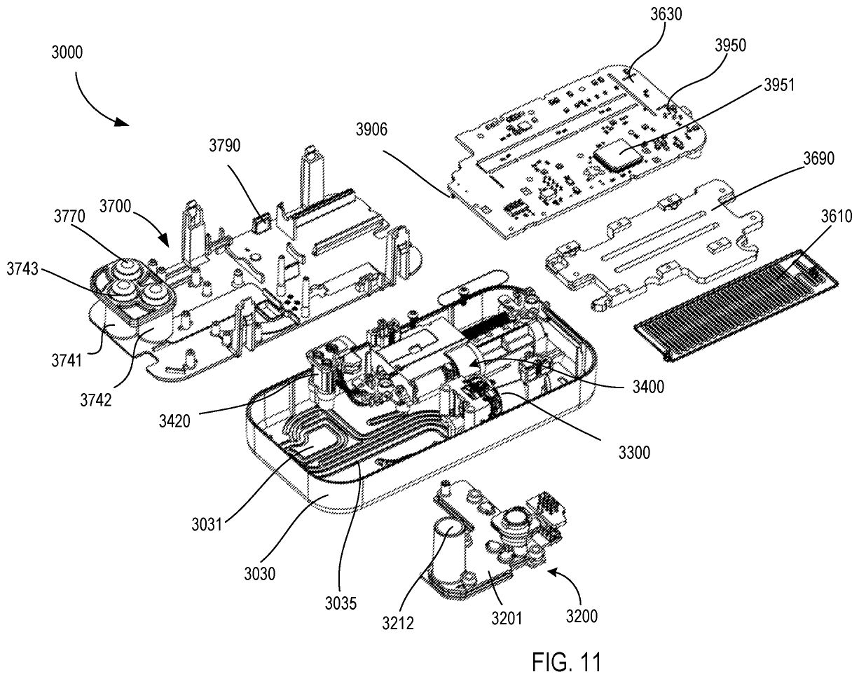

The lower housing 3030 includes a support substrate 3031 and defines a volume within which the modules and or components of the device 3000 are disposed. As shown in FIG. 14, the support substrate 3031 defines a series of flow channels 3035 (in some examples, the support substrate defines a manifold) that are aligned with flow channels of other components within the device to allow for fluid transfer between the various modules and components without the need for tubing, clamps and the like. Specifically, as shown in FIG. 33, the bottom of the reagent module 3700 defines a series of flow channels 3735 that correspond to the flow channels 3035 in the support substrate 3031 and thus facilitate transfer of fluids within the device (see FIG. 14). When the reagent module 3700 is coupled to the support substrate 3031, a portion of this assembly can be referred to (and function as) a flow (or reagent) manifold. Although the bottom plate 3031 is described as being within (or a portion of) the lower housing 3030, in some embodiments, the modules, including the reagent module 3700, can be assembled to the support substrate 3031 (as shown in FIGS. 12 and 13), and the module assembly can then be assembled into the lower housing 3030. Thus, in addition to including the flow channels 3035 and forming a portion of the flow manifold, the support substrate 3031 also supports the modules within the device (including the amplification module 3600 and the detection module 3800).

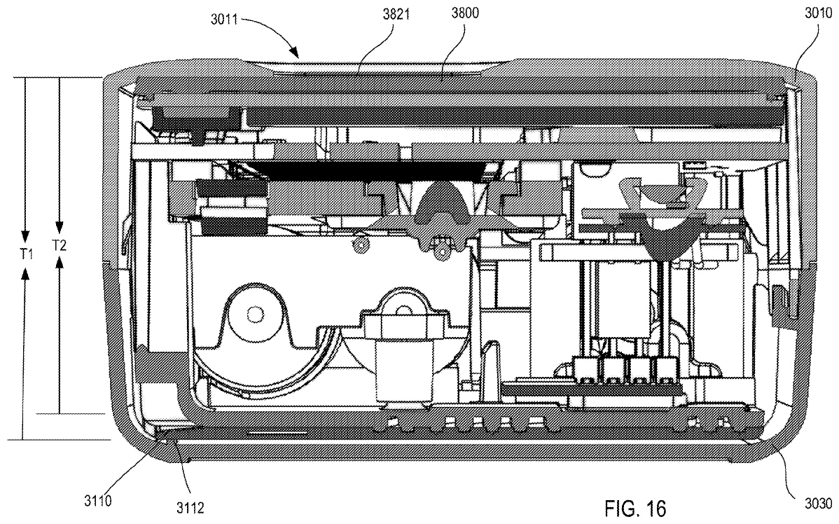

The support substrate 3031 includes a set of biasing members 3110 suitable to apply a biasing force on the detection module 3800 causing the detection surface 3821 to be biased against the top housing 3010. As shown in FIG. 16, one or more of the modules or components of the test device 3000 are biased against one or more surfaces of the housing 3001. Specifically, the detection surface 3821 is biased against the external wall of the top housing 3010 such that the visible signal produced at the detection surface 3821 is clearly and repeatably visible through the detection opening 3011. Specifically, the support substrate 3031 is biased towards the top housing 3010 that defines the detection opening 3011. Thus, the support substrate 3031 can apply a biasing force, via the biasing members 3110, such that components of one or more modules are pressed against the top housing 3010. Specially, the biasing force causes the detection surface 3821 to be biased against the housing 3001. In this manner, the biasing force (or preload) maximizes the viewing angle of the detection surface 3821 by ensuring that the detection surface is as close to the detection opening 3011 as possible. In situations in which the detection surface is spaced apart from of the upper housing, the viewing angle is negatively affected. For example, in such situations shadows can be cast on the detection surface 3821 due to the gap. Thus, the biasing minimizes the distance between the detection module 3800 and the detection opening 3011, which minimizes shadows and optical aberrations and improves readability of the test results. In some embodiments, the detection opening 3011 is defined by a beveled opening in the top housing 3010, further reducing negative viewing effects of the detection surface (e.g., shading).

The biasing may be applied against one or more of the modules within the housing 3001 or one or more components of the modules in any suitable manner, in any suitable direction (within the housing), and against any of the housing surface. For example, although the support substrate 3031 engages one or more biasing members 3110 such that the support substrate 3031 is biased in one direction (i.e., towards the top housing 3010) in the housing, in other embodiments, any of the modules described herein can be biased towards the bottom housing 3030 or against the sides of the housing 3001. In other embodiments, a device can include multiple biasing members, some of which can bias a first module (e.g., the detection module 3800) in a first direction (e.g., towards the top housing 3010) and others of which can bias a second module (e.g., the reagent module 3700) in a different direction.

In some embodiments, the biasing force urges an outer surface of the module or component into contact with an inner surface of the top housing 3010 to form a contact region surrounding the detection opening 3011. For example, the detection surface 3821 can be biased against the top housing 3010 at or near the detection opening 3011 (as described above) forming a contact region around the detection opening 3011. In addition to enhancing viewing, having a tight press between the detection surface 3821 and the top housing 3010 reduces or prevents leakage through the detection opening 3011.

The support substrate 3031 can be any suitable structure within the housing 3001. In some examples, the support substrate 3031 can form a component of any of the modules or components described herein (e.g., a flow manifold discussed herein). In some examples, the support substrate defines a manifold having flow channels 3035 (otherwise referred to as a flow manifold) In other examples, the support substrate 3031 can be a separate component (e.g., a supporting structural component) that extends between different modules or supports a single module. As an example, FIGS. 12-13 illustrate the support substrate 3031 supporting both the sample preparation module 3200, the amplification module 3600, and the detection module 3800 (as well as the detection surface 3821). In other examples, however, the support substrate 3031 can support just the sample preparation module 3200. Alternatively, the support substrate 3031 can support just the detection module 3800. Additionally or alternately, the support substrate 3031 can support other modules such as the amplification module 3600.

Although the biasing members 3110 are shown as being deformable tabs formed as a part of the support substrate 3031, in other examples, the biasing members can include one or more springs or other deformable members (e.g., compression, extension, torsion, constant force, Belleville, drawbar, volute, flat, gas, etc.). Thus, the biasing members 3110 can include any suitable structure that applies a biasing force between two components such as, the housing, a module, and/or the support substrate. Here, the biasing members 3110 can be a molded flexible protrusion from the support substrate such as a tab. Moreover, although the device 3000 is shown as including multiple biasing member, in other embodiments, a device can include only a single biasing member. In some examples, the biasing members 3110 can be integral to one or more components of the device 3000.

In some embodiments, the housing 3001 (or any other suitable structure within the device 3000) includes any suitable structure (e.g., shoulders, mounts, etc.) to apply a counterforce against the biasing members 3110. For example, as shown in FIGS. 14 and 16, the bottom housing 3030 includes one or more mounts 3112. The mounts 3112 define an attachment point or support surface against which the biasing members 3110 are pressed or engaged. The mounts 3112 can be any suitable feature for locating, retaining, anchoring, or otherwise supporting feature for the biasing members. As shown in FIGS. 14 and 16, when the modules are installed into the bottom housing 3030, the biasing members 3110 contact mounts (e.g., shoulders) 3112. The mounts (e.g., shoulders) 3112 apply a resistant force to the biasing members 3110 forcing the support substrate 3031 upward (toward the top housing 3010).

Referring back to FIG. 13 and in accordance with some embodiments, the biasing members 3110 produce a biasing range or distance (T1 minus T2). The biasing range is the unencumbered travel of the biasing mechanism in relationship to the stack up height T1 of the supported modules and/or components. Thus, the biasing range is the range of travel for the biasing mechanism that allows the support substrate 3031 (and the modules supported thereon) to move to a position, when not restricted by supported modules or components, that is at a substrate distance T2 from the opposing interior housing surface. In some embodiments, the stack up height T1 is greater than the substrate distance T2. In this way, the biasing members 3110 are able to maintain a bias of the highest component in the module/component stack against the interior surface of the top housing 3010 creating a component preload. In the example illustrated in FIG. 13, the detection module 3800 is preloaded against the interior surface of the upper housing forming a contact region 3823 (see FIG. 15) between the two.

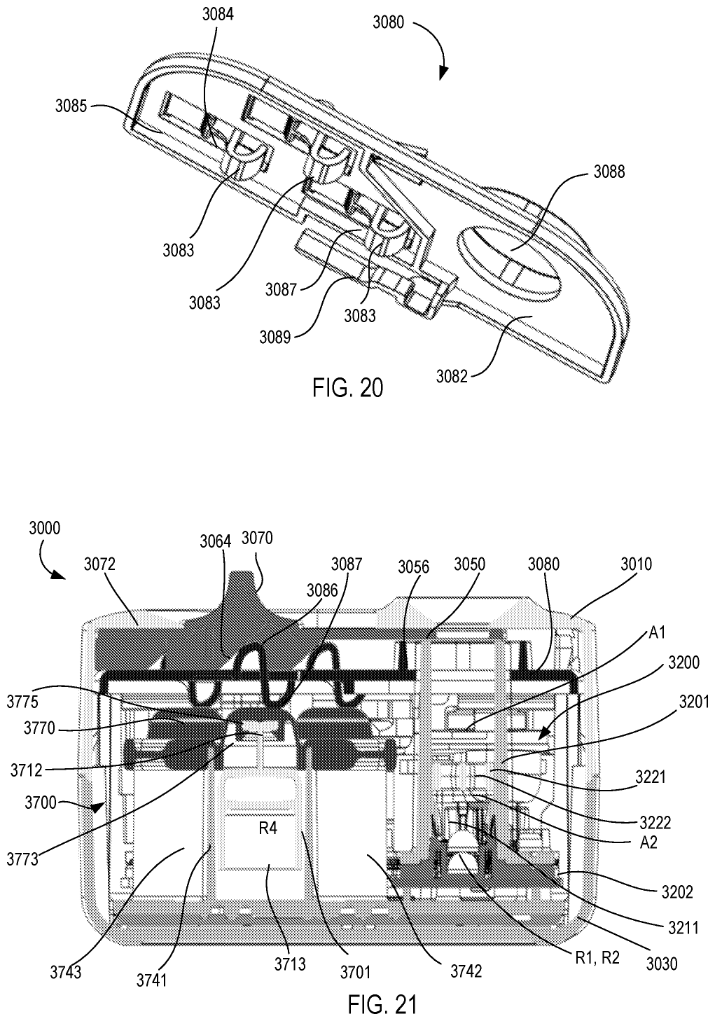

As shown in FIGS. 16-18, the lid 3050 includes a first (or outer) surface 3051 and a second (or inner) surface 3052. Referring to FIGS. 21 and 22, the lid 3050 is coupled to the housing 3001 and is positioned between the top housing 3010 and the flexible plate 3080. As described below, the lid 3050 and the flexible plate 3080 collectively actuate the reagent module 3700 when the lid 3050 is moved relative to the housing 3001. The guide rails 3056 of the lid 3050 are configured to engage with the flexible plate 3080, and thus also facilitate sliding movement of the lid 3050 (relative to the flexible plate 3080). As shown by the arrow GG in FIG. 22, the lid 3050 is configured to move relative to the housing 3001 from a first (or opened) position (FIG. 21) to a second (or closed) position (FIG. 22).

The lid 3050 is configured to perform a variety of functions when moved relative to the housing 3001, thereby facilitating actuation of the device 3000 via a single action. Specifically, the lid 3050 includes a cover portion 3053, a switch portion 3060, and three reagent actuators 3064. The cover portion 3053 includes a cover surface 3057 and defines an input opening 3054. When the lid 3050 is in the opened position (see e.g., FIGS. 8, 9, and 41A), the input opening 3054 is aligned with each of the sample input opening 3021 of the top housing 3010 and the input opening 3212 of the sample preparation module 3200 and thus provides an opening through which the biological sample S1 can be conveyed into the device 3000. The cover surface 3057 is a flat surface that covers (or obstructs) each of the sample input opening 3021 of the top housing 3010 and the input opening 3212 when the lid is in the closed position (see FIGS. 41B and 41C). Specifically, the cover surface 3057 is spaced apart from the input opening 3212 and/or the sample input opening 3021 when the lid 3050 is in the opened position, but covers the input opening 3212 and/or the sample input opening 3021 when the lid 3050 is in the closed position. In some embodiments, when the lid 3050 is in the closed position, the cover surface 3057 is spaced apart from the sample input reservoir 3211 thereby forming an air gap between the lid 3050 and the input reservoir 3211. In use, air that is conveyed into the input reservoir to aid in mixing the biological solution with the reagents can be vented out of the input reservoir via the air gap. The lid 3050 does not seal closed the housing opening 3021 and/or the sample input opening 3212. This air gap can limit pressurization of the input reservoir when the device is active, thereby allowing flow of air into the sample input reservoir 2211 to enhance mixing. In other embodiments, however, the cover surface 3057 includes a seal, gasket, or other material to fluidically isolate the sample input reservoir 3211 (of the sample preparation module 3200) when the lid 3050 is in the closed position. For example, a device 3000 having a

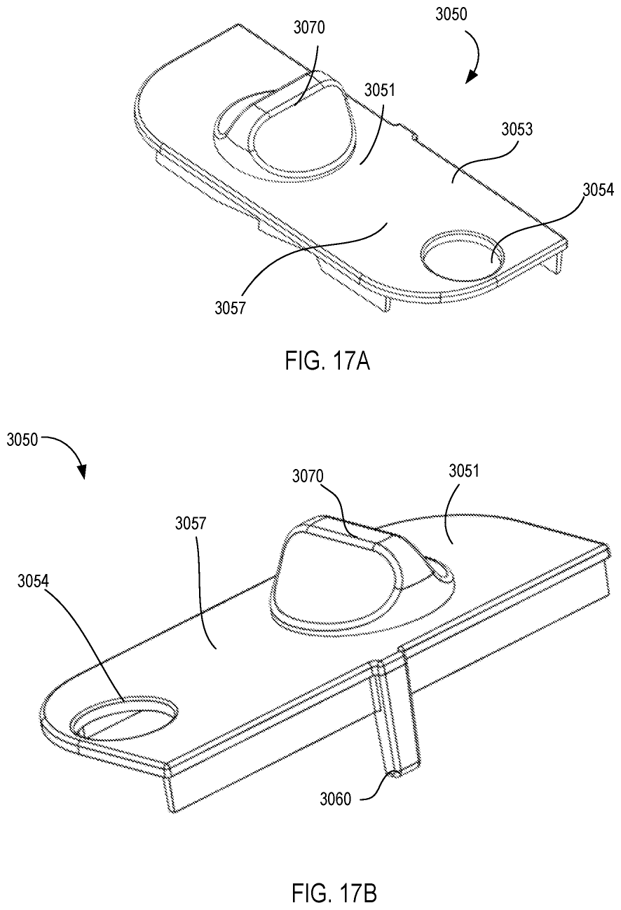

In addition to covering the input opening 3212, closing the lid 3050 also actuates other mechanisms within the device 3000. Specifically, as shown in FIGS. 17 and 18, the switch portion 3060 includes a protrusion that actuates the switch 3906 (FIG. 11) when the lid 3050 is moved from the opened position to the closed position. Specifically, the switch portion 3060 indirectly actuates the switch 3906 by deforming a corresponding switch portion 3089 of the flexible plate 3080. When the lid 3050 is moved to the closed position, the switch portion 3060 slides within the gap that separates the corresponding switch portion 3089 from the body of the flexible plate 3080, thereby deforming the corresponding switch portion 3089 into an outward position. When in its outward position, the corresponding switch portion 3089 actuates the switch 3906. When the switch is actuated (i.e., is moved from a first state to a second state), power from the power source (e.g., the power source 3905) can be provided to the electronic control module 3950 and any other components within the device 3000 that require power for operation. For example, in some embodiments, power is provided to any of the heaters (e.g., the heater 3230 of the sample preparation module 3200, the heater 3630 of the amplification module 3600, and the heater 3840 of the detection module 3800) directly or via the electronic control module 3950. For example, this allows the heater 3230 to begin preheating for a lysis operation after the lid 3050 is closed and the device 3050 is coupled to the power source 3905 without requiring further user action. Although the switch 3906 is shown as being a rocker switch that is actuated directly by the protrusion of the switch portion 3060, in other embodiments, the switch 3906 (and the corresponding switch portion 3060) can be any suitable switch that performs the functions described herein. For example, in some embodiments, the switch can be an isolation member that electrically isolates the power source 3905 from the remaining components of the electronic control module 3950. In such embodiments, the switch portion 3060 can be coupled to, and can remove, the isolation member (thereby electrically coupling the power source 3905 to the electronic control module 3950). In other embodiment, the switch portion 3060 is the isolation member, and no separate switch is included in the electronic control module 3950.

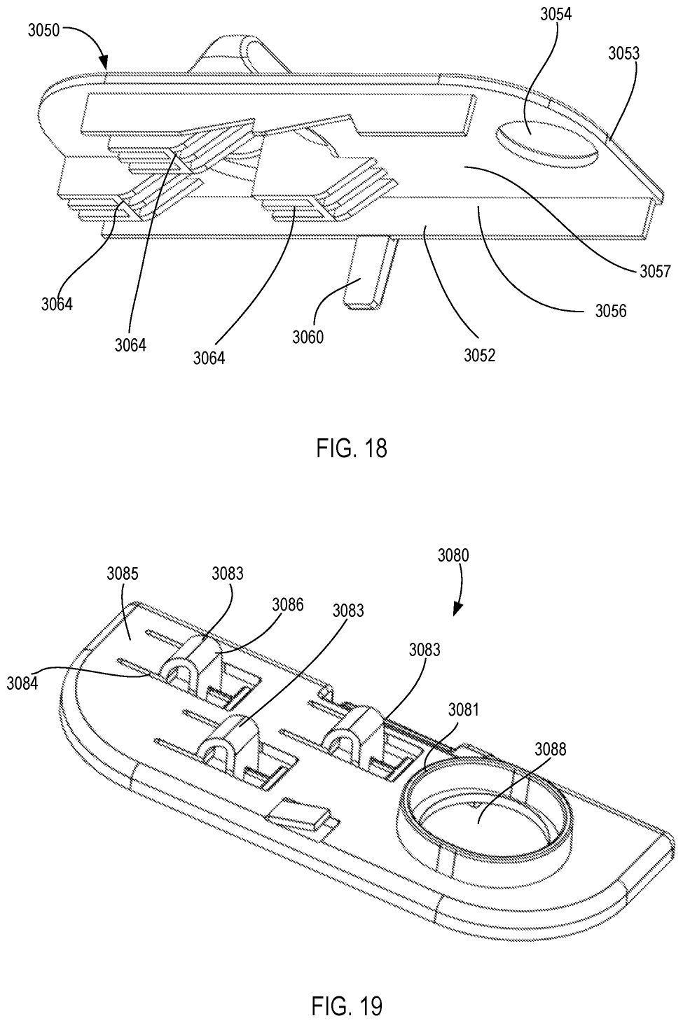

Referring to FIGS. 18 and 19, the reagent actuators 3064 include a series of ramped surfaces that exert an actuation force on a corresponding set of deformable actuators 3083 of the flexible plate 3080 when the lid 3050 is moved from the opened position (FIG. 21) to the closed position (FIG. 22). In this manner, the reagent actuators 3064 (and the deformable actuators 3083 of the flexible plate 3080) cause the reagent to be released from the sealed reagent containers within the reagent module 3700, as described in more detail below.

The outer surface 3051 of the lid 3050 includes a handle 3070. The handle 3070 extends through the actuator opening 3022 of the top housing 3010 and provides a structure that can be manipulated by the user to move the lid 3050 from the opened position to the closed position. The handle 3070 can be moved in the direction shown by the arrow GG in FIG. 22 to close the lid 3050. In some embodiments, the lid 3050 is irreversibly locked after being closed to prevent reuse of the device 3000 and/or the addition of supplemental sample fluids.

The flexible plate 3080 (shown in FIGS. 19 and 20) includes an outer surface 3081 and an inner surface 3082. As described above, the lid 3050 is movably disposed between the top housing 3010 and the flexible plate 3080. Similarly stated, the outer surface 3051 of the lid 3050 faces the inner surface of the top housing 3010 and the inner surface 3052 of the lid 3050 faces the outer surface 3081 of the flexible plate 3080. The flexible plate includes three deformable actuators 3083, each of which is aligned with a corresponding reagent actuator 3064 of the lid 3050 and one of the reagent containers 3701, 3702, 3703. Thus, when the lid 3050 is moved relative to the housing 3001, the reagent actuators 3064 and the deformable actuators 3083 actuate the reagent module 3700. In particular, as described in detail below, the reagent actuators 3064 and the deformable actuators 3083 move the reagent containers 3701, 3702, 3703 within the reagent manifold 3730 to release the reagents that are sealed within the containers.

The flexible plate 3080 defines a channel 3084 for the surrounds at least three sides of each of the deformable actuators 3083. Thus, each of the deformable actuators 3083 remains coupled to the flexible plate 3080 by a small strip of material (or living hinge) 3085. Accordingly, when the reagent actuator 3064 exerts an inward force on the outer surface 3086 of deformable actuator 3083, the deformable actuator bends or deforms inwardly towards the reagent module 3700 as shown by the arrow HH in FIG. 22. This action causes the inner surface 3087 of each of the deformable actuators 3083 to apply an inward force on the reagent containers (and the deformable support member 3770) thereby moving the reagent containers downward within the reagent manifold 3730, as shown by the arrow HH in FIG. 22.

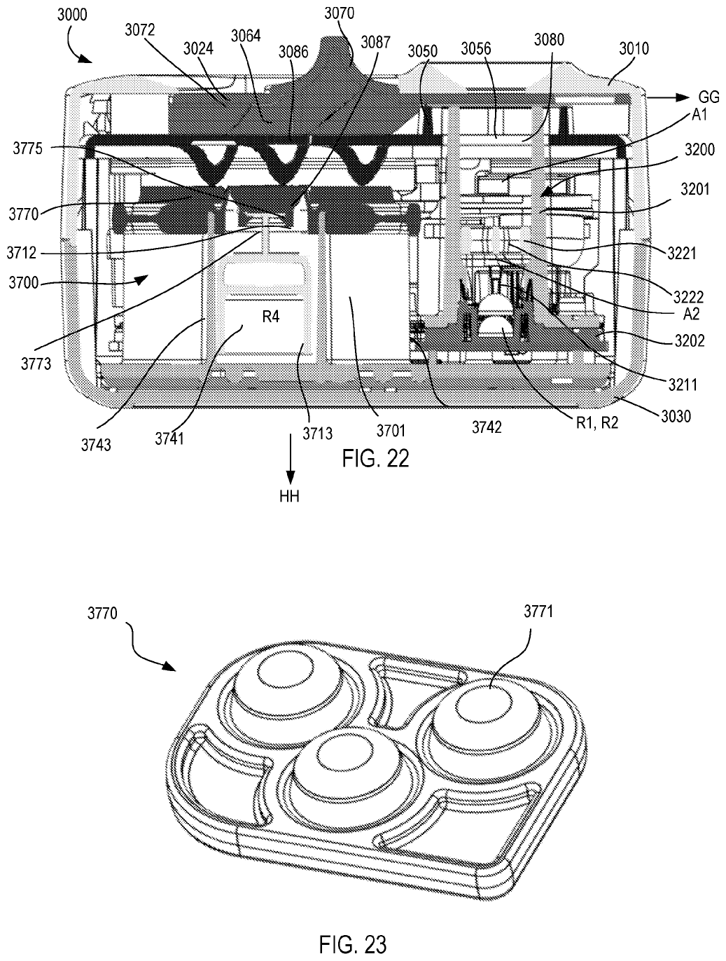

Referring to FIGS. 21, 22, 32, and 33, the reagent module 3700 includes a reagent manifold (or housing) 3730, three reagent containers 3701, 3702, 3703, and a deformable support member 3770) (see FIGS. 23 and 24). The reagent module 3700 provides mechanisms for long-term storage of reagents within the sealed reagent containers, actuation of the reagent containers to release the reagents from the reagent containers for use during the methods described herein. In addition to providing storage and actuating functions, the reagent module 3700 also provides fluid interconnections to allow the reagents and/or other fluids to be conveyed within the device 3000. Specifically, as described herein, the reagent module 3700 is fluidically coupled to the fluid transfer valve 3300 in a manner that allows selective venting, fluid coupling, and/or conveyance of the reagents and substances within the device 3000.

The reagent module 3700 stores packaged reagents, identified herein as reagent R4 (a dual-purpose blocking and wash solution), reagent R5 (an enzyme reagent), and reagent R6 (a substrate), and allows for easy un-packaging and use of these reagents in the detection module 3800. As shown schematically in FIG. 7B, the reagent module 3700 includes a first reagent container 3701 (containing the reagent R4), a second reagent container 3702 (containing the reagent R5), and a third reagent container 3703 (containing the reagent R6). Each of the reagent containers includes a connector at a first end portion and a frangible seal at a second, opposite end portion. Specifically, as shown in FIGS. 21 and 22, the first reagent container 3701 includes a connector 3712 and a frangible seal 3713. The connector 3712 connects the first reagent container 3701 to the mating coupling portion 3775 of the deformable support member 3770. The frangible seal 3713 is any suitable seal, such as, for example, a heat-sealed BOPP film (or any other suitable thermoplastic film). Such films have excellent barrier properties, which prevent interaction between the fluids within the reagent container and external humidity, but also have weak structural properties, allowing the films to be easily broken when needed. When the reagent container is pushed into the puncturers, as described below; the frangible seal breaks, allowing the liquid reagent to flow into the appropriate reagent reservoir when vented by the fluid transfer valve 3300. Although only the details of the first reagent container 3701 are shown and described herein, the second reagent container 3702 and the third reagent container 3703 have similar structure and function.

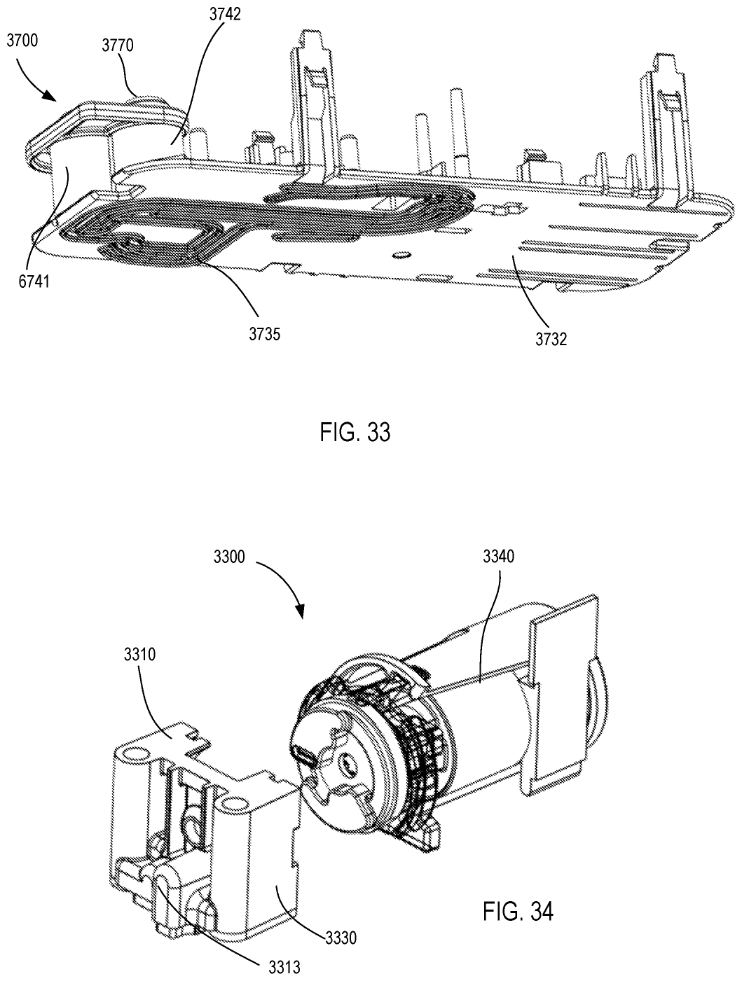

Referring to FIGS. 32 and 33, the reagent manifold 3730 includes a top (or outer) surface 3731 and a bottom (or inner) surface 3732. As described above, the support substrate 3031 is coupled to the bottom surface 3732, thus collectively forming the reagent (or fluid) manifold The reagent manifold 3730) includes three reagent tanks extending from the top surface 3731 and within which the reagent containers are disposed. Specifically, the reagent manifold includes a first reagent tank 3741 within which the first reagent container 3701 is disposed, a second reagent tank 3742 within which the second reagent container 3702 is disposed, and a third reagent tank 3743 within which the third reagent container 3703 is disposed. The reagent housing 3730 includes a pair of puncturers in the bottom portion of each reagent tank. The puncturers are configured to pierce the frangible seal of the respective reagent container when the reagent container is moved downward within the reagent housing 3730. Similarly stated, the reagent housing 3730 includes a set of puncturers that pierce a corresponding frangible seal to open a corresponding reagent container when the reagent module 3700 is actuated. The puncturers define a flow path that places the internal volume of the reagent container and/or the reagent tank in fluid communication with an outlet port of the reagent module 3700 after the frangible seal is punctured.

The deformable support member 3770 includes an outer surface 3771 and an inner surface 3772. As described above, the outer surface 3771 includes actuation regions that are aligned with one of the deformable actuators 3083 of the flexible plate 3080. The inner surface 3772 includes three seal portions 3773 and three coupling portions 3775. As shown in FIGS. 21 and 22, each of the seal portion 3773 is coupled to the reagent housing 3730) to fluidically isolate the internal volume (i.e., the reagent reservoir) of the corresponding reagent tank. The coupling portions 3775 are each coupled to one of the connectors of the corresponding reagent container. As an example, one of the seal portions 3773 is coupled to the top portion of the first reagent tank 3741 to fluidically isolate (or seal) the internal volume of the first reagent tank 3741. Additionally, one of the coupling portions 3775 is coupled to the connector 3712 of the first reagent container 3701.

The deformable support member 3770 is configured to deform from a first configuration (FIG. 21) to a second configuration (FIG. 22) in response to an actuation force exerted thereon (e.g., by the deformable actuator 3083). Moreover, the deformable support member 3770) is biased in the first (or undeformed) configuration. In this manner, the deformable support member 3770) supports each of the reagent containers in a “storage state” when the deformable support member 3770) is in the first configuration. Similarly stated, the deformable support member 3770) maintains the puncturer spaced apart from the frangible seal 3713 of the reagent container 3701 when the deformable support member is in the first configuration.

When the lid 3050 is moved, the downward force exerted by the deformable actuators 3083 cause the deformable support member 3770) to transition to the second (or deformed) configuration (FIG. 22). Similarly stated, when the downward force is sufficient to overcome the opposite, biasing force of the deformable support member 3770, the deformable support member 3770) is transitioned to the second configuration, as shown by the arrow HH in FIG. 22. This causes each of the reagent containers to move downward within the corresponding reagent tank, bringing the puncturers into contact with the frangible seal of each reagent container. Similarly stated, when the deformable support member 3770) is in the second configuration, the puncturers pierce the frangible seal 3713 of the reagent container 3701, thereby release the reagent R4 from within the reagent container 3701. Although FIG. 22 shows the actuation for only the first reagent container 3701, when the reagent module 3700 is actuated, each of the first reagent container 3701, the second reagent container 3702, and the third reagent container 3703 are actuated in this manner. Thus, in addition to covering the sample input opening and providing power to the electronic control module 3950, closing the lid 3050 also actuates all of the reagent containers.

Although shown as including three reagent containers, in other embodiments, the reagent module 3700 (or any of the reagent modules described herein) can have any suitable number of reagent containers. For example, in some embodiments, a reagent module can include only one reagent container, similar the reagent module 2700 described herein.

Referring to FIG. 32, the outer surface 3731 of the reagent manifold 3730) includes a set of valve fluid interconnects 3736, a set of mixing reservoir fluid interconnects 3737, and a set of detection module fluid interconnects 3738. Each of these fluid interconnects is coupled to one of the reagent tanks and/or other components within the device 3000 by the flow channels 3035, 3735 defined in support substrate 3031 and the inner surface 3732, respectively. Additionally, the outer surface 3731 includes multiple mounting clips 3790. Thus, the valve fluid interconnects 3736 (and the appropriate channels 3735) provide fluidic coupling to the fluid transfer valve 3300, which is coupled to the top surface 3731 by one of the clips 3790. The mixing reservoir fluid interconnects 3737 (and the appropriate channels 3735) provide fluidic coupling to the mixing assembly 3250, which is coupled to the top surface 3731. The detection module fluid interconnects 3738 (and the appropriate channels 3735) provide fluidic coupling to the detection module 3800.

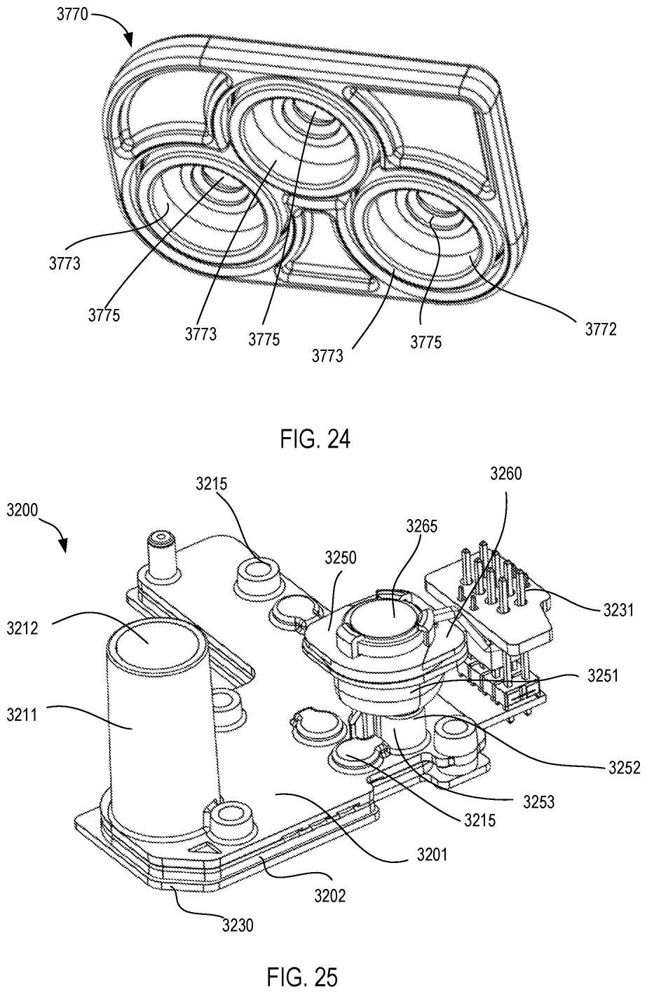

FIGS. 25-29 show various views of the sample preparation module 3200. As described herein, the sample preparation module 3200 can perform any or all of A) receiving the biological sample S1, B) mixing the biological sample with desired reagents (e.g., a positive control reagent R1 and/or a reverse transcriptase R2), C) performing lysing operations to release target RNA from the biological sample S1, D) performing a reverse transcription reaction to produce cDNA, and E) heating the resulting solution to inactivate the reverse transcriptase. Thus, in some embodiments, the sample preparation module enables an efficient, fast RT-PCR to be performed within a single environment or module. By eliminating the need for external sample preparation and a cumbersome instrument, the device 3000 is suitable for use within a point-of-care setting (e.g., doctor's office, pharmacy or the like), a decentralized location, or at the user's home and can receive any suitable biological sample S1. The biological sample S1 (and any of the input solution described herein) can be, for example, blood, urine, male urethral specimens, vaginal specimens, cervical swab specimens, and/or nasal swab specimens gathered using a commercially available sample collection kit.

The sample preparation module 3200 includes a top body 3201, a bottom body 3202, a heater 3230, and a mixing assembly 3250. The top body 3201 and the bottom body 3202 can be referred to collectively as a sample preparation housing, a flow member or a reverse transcription chamber. Although the flow member is shown as being constructed from two pieces (the top body 3201 and the bottom body 3202) that are coupled together, in other embodiments, the flow member can be monolithically constructed. The sample preparation housing (i.e., the top body 3201 and the bottom body 3202) define a sample input opening 3212, a input reservoir 3211, and a serpentine flow channel 3214. In some embodiments, the top body 3201 and/or the bottom body 3202 can define one or more vents. Such vents can allow air to flow into or out of the sample preparation module 3200 (including the input reservoir 3211 and the serpentine flow channel 3214) as sample is conveyed into and/or out of the sample preparation module 3200. Additionally, the top body 3201 includes a set of fluid interconnects 3215 that allow for fluidic coupling of the sample preparation module 3200 to the fluid transfer valve 3300 and other components within the device 3000.

The sample input opening 3212 is an opening through which the input reservoir 3211 can be accessed. As described above, when the lid 3050 is in the opened position, the biological sample S1 can be conveyed into the input reservoir 3211 via the sample input opening 3212. The input reservoir 3211 is a volume within which the biological sample S1 can be mixed with reagents and also heated. For example, in some embodiments the biological sample S1 can be collected in the input reservoir 3211 and mixed with either or both of a control organism (identified as reagent R1) and a reverse transcriptase (identified as reagent R2). The control organism and the reverse transcriptase can each be lyophilized or otherwise in solid form. Moreover, the reagents R1 and R2 can be secured within the input reservoir 3211 to prevent the reagents R1 and R2 from inadvertently falling out of the device 3000, for example during storage, transportation, or use. For example, as illustrated in FIGS. 21, 22, 26A, 27, and 28, in accordance with some embodiments, the sample preparation module 3200 includes a retention screen 3221. The retention screen 3221 is positioned to separate the input reservoir 3211 into a first portion A1 and a second portion A2. The sample input opening 3021 and the sample input opening 3212 each extend into the first portion A1. The second portion A2 is separated from the sample input opening 3021 and the sample input opening 3212 by the retention screen 3221. The second portion A2 contains a solid reagent (e.g., R1, R2). In this manner, the retention screen 3221 limits the movement of the solid reagent. In particular, the solid reagent is limited from exiting the sample input opening 3021 and/or the sample input opening 3212 by the retention screen 3221. The sample preparation module 3200 is configured to receive a biological sample and to mix the biological sample with the solid reagent R1, R2 to form an input solution S2 containing a target molecule.

In accordance with some embodiments, the retention screen 3221 defines one or more apertures 3222. The one or more apertures 3222 are sized to allow the biological sample S1 to flow through the retention screen 3221 from the first portion A1 of the input reservoir 3211 to the second portion A2 of the input reservoir 3211. In one example, the apertures are large enough to allow the biological sample S1 to flow through the retention screen 3221 from the first portion A1 into the second portion A2. Additionally or alternatively, the apertures are large enough to allow a mixing fluid (e.g., air and their bubbles in the biological sample as discussed herein) to flow from the second portion A2 into the first portion A1. The one or more apertures 3222 are sized to limit the ability of the solid reagent (e.g., R1, R2) within the second portion A2 of the input reservoir 3211 from exiting the input reservoir 3211 via the sample input opening 3021. Specifically, the apertures are small enough to retain the solid reagent (e.g., R1, R2) within the second portion A2. For example, the aperture area is less than the solid reagent cross section.

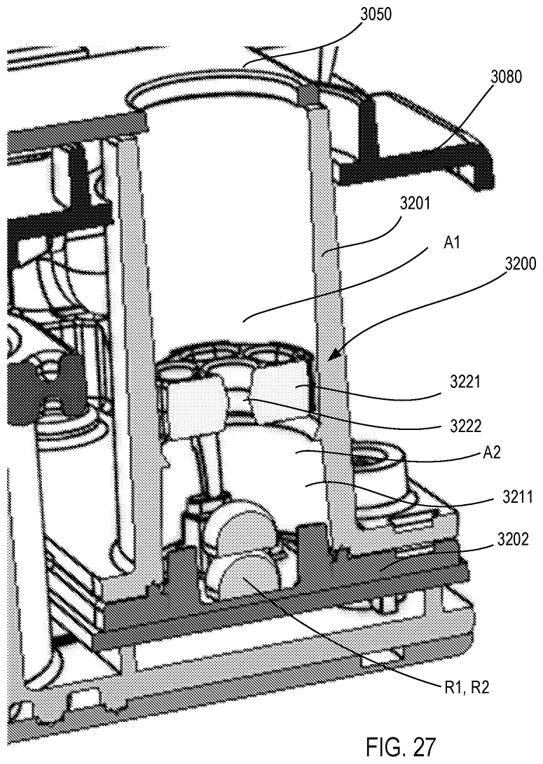

Referring to FIGS. 27 and 28 the sample preparation module 3200 incudes a coupling protrusion 3223 within the input reservoir 3211. The retention screen 3221 is coupled to the coupling protrusion 3223. The protrusion 3223 defines the height of the retention screen 3221 within the input reservoir 3211. Similar to the H2 discussed above, the height here is suitable to allow for the solid reagent (e.g., R1, R2) and the biological sample to mix by bubbling a fluid (e.g., air) therethrough.

In some embodiments, the reagent R1 is a positive control organism, such as Aliivibrio fischeri. N. subflava, or any other suitable organism. Specifically, Aliivibrio fischeri is suitable because it is gram negative, nonpathogenic, bio safety level 1, not harmful to the environment, and is extremely unlikely to be found on a human. The positive control surface within the detection module contains capture probes for both the control organism (e.g., A. fischeri) as well as each of the target organisms. This arrangement ensures that the positive control surface always produces color if the device functions correctly. If only the control organism were present, a very strong positive for one of the target organisms could “swamp out” or “outcompete” the amplification of the control organism during PCR. Under such circumstances, the positive control spot would not produce a color change which would be confusing for the user. This arrangement facilitates the detection method and the device 3000 being operated by a user with minimal (or no) scientific training, in accordance with methods that require little judgment.

In some embodiments, the reagent R2 contains the reverse transcriptase enzymes and other constituents to facilitate the RT-PCR methods described herein. For example, in some embodiments, the reagent R2 includes the salts needed to create the correct buffering environment for the RT-PCR. In some embodiments, the reagent R2 can include one or more reducing agents. In some embodiments, the reducing agent is lithium, sodium, potassium, aluminum amalgam, lithium aluminum hydride, sodium borohydride, sodium cyanoborohydride, lithium tri-butoxy aluminum hydride, dibutoxy aluminum hydride, lithium triethyl borohydride, tris(2-carboxyethyl)phosphine (TCEP), dithiothreitol (DTT), mercaptoethanol, cysteine, thioglycolic acid, cysteamine, glutathione, N-acetylcysteine (NAC), β-mercaptoethanol, sodium dodecyl sulfate (SDS), sodium sulfite, ammonium sulfamate, sodium bisulfite, dithionite metabisulfite sulfur dioxide, vitamin C, ascorbic acid, dimethylsulfoxide (DMSO), or any combination thereof. In some embodiments, the dried particles comprise NAC. Although the input reservoir 3211 is shown as including two reagent pellets (R1, R2), in other embodiments, the input reservoir 3211 only includes one reagent pellet, or alternatively, can include more than two reagent pellets.

The reagents are formulated to dissolve in the biological sample within the input reservoir 3211. In some embodiments, the mixing can be enhanced by any of the methods described herein, including the method 10 described above. In some embodiments, air can be conveyed into the input reservoir 3211 to mix the biological sample and the reagents R1, R2 therein. Specifically, upon actuation of the device, air can be conveyed from the fluidic drive module 3400 through the serpentine channel 3214, through the inlet opening 3213, and into the input reservoir 3211. The air can be present in the fluidic drive module 3400 and the valve assembly 3300 can be placed into a configuration to allow this retrograde flow of air into the input reservoir 3211. The air can be conveyed at a rate and for a time period sufficient to facilitate dissolution and/or mixing of the solid reagents within the biological sample. For example, in some embodiments, the air can be conveyed in a manner to produce bubbles and/or turbulence with the second portion A2 of the input reservoir 3211.

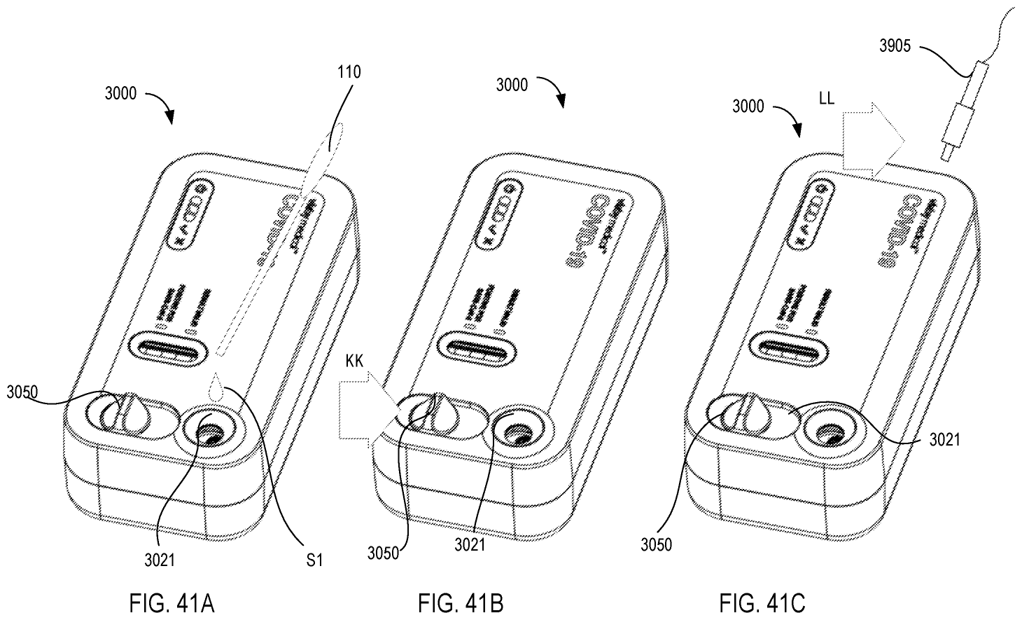

In accordance with some embodiments, the mixing in the input reservoir 3211 between the biological sample and the solid reagents can be passive and additionally or alternatively include active mixing similar to those protocols discussed with reference to FIGS. 2-5 discussed above. In one example of active mixing, the fluid is injected into the input reservoir 3211 for a suitable time to mix the biological sample and the solid reagent R1, R2 such that when the solution is pulled from the input reservoir the reagents are mixed into the biological sample in concentrations that remain generally consistent through the process. In one example, the air pushed through the input reservoir 2211 between 10-30 μL/s. In one example, the air is pushed through the input reservoir 2211 at between 15-25 μL/s. In one example, the air is pushed through the input reservoir at about 20 μL/s. This last example is illustrated in FIG. 42. FIG. 42 illustrates experimental data in which the concentration factor of a red dye and a blue dye in a solution is illustrated based on 20 aliquots. The concentration factor is the concentration normalized at 1. This means that the average of the concentration factors for each aliquot for through the entire solution will equal 1. Thus, if aliquots in a portion of the total solution tend to have a low concentration factor then aliquots in another portion of the total solution will offset them such that the average of all aliquots will be at or about 1. For, consistency of mixture and concentration, each reagent (here represented by the red and blue die) would have a similar concentration factor to the other reagent and be close to 1. As shown in FIG. 42 the forced air mixing in the input reservoir illustrates this result by having the concentration factors for the red and blue dye remain close to one another and reside between concentration factors of 0.75 and 1. Passive mixing tends to result in a 70% variance in the concentration of the extracted fluid compared to the target concentration. Forced air mixing reduces this variation by at least 10-15%.

As described herein, the apertures 3222 of the retention screen 3221 can be sized to permit the air to vent from the second portion A2. Moreover, as described above, the cover surface 3057 is spaced apart from the sample input reservoir 3211 thereby forming an air gap between the lid 3050 and the input reservoir 3211 through which the air can be vented. As discussed above, the lid 3050 does not seal closed the housing opening 3021 and/or the sample input opening 3212. This air gap can limit pressurization of the input reservoir when the device is active, thereby allowing flow of air into the sample input reservoir 3211 to enhance mixing.

In some embodiments, the biological sample can be heated within the input reservoir 3211 to lyse the cells within the biological sample S1 and further lyse (or release) the target RNA from any viruses contained with the biological sample S1. In other words, the biological sample S1 can be heated to both break apart the cells and also disrupt the viruses therein to release target RNA for detection. Specifically, the heater 3230 is coupled to the sample preparation housing and/or the bottom body 3202 such that a first portion of the heater 3230 can convey thermal energy into the input reservoir 3211. The first portion of the heater 3230 can maintain the biological sample S1 at any suitable temperature and for any of the time periods described herein. For example, in some embodiments, the biological solution can be maintained at a temperature within a lysing temperature range to release a ribonucleic acid (RNA) molecule. The lysing temperature range can be, for example, between about 25 C and about 70 C. In other embodiments, the lysing temperature range can be between about 25 C and about 50 C.

Referring to FIG. 27, which shows a top view cross-section of the sample preparation housing, the input reservoir 3211 is in fluid communication with the serpentine flow channel 3214, via the inlet opening 3213. In this manner, the lysed biological sample that is mixed with the RT enzyme (also referred to as a reverse transcription solution) can flow from the input reservoir 3211 through the serpentine flow channel 3214. More specifically, when a pressure gradient is applied across the inlet opening 3213 and the output opening 3215 (e.g., via the fluidic drive module 3400), the reverse transcription solution can flow from the input reservoir 3211 (first volume) through the serpentine flow channel 3214. The serpentine channel provides a high surface area to volume ratio, and thus allows for rapid RT-PCR and inactivation of the lysis and/or RT enzymes in the solution. The reverse transcription solution can flow in a direction opposite the direction that the mixing air (described above) is conveyed into the input reservoir.

In use, the reverse transcription solution can be heated as it flows through the serpentine flow channel 3214 to perform RT-PCR and to optionally further inactivate the enzymes. Specifically, the heater 3230 is coupled to the sample preparation housing and/or the bottom body 3202 such that a second portion of the heater 3230 can convey thermal energy into the serpentine flow channel 3214. The second portion of the heater 3230 can maintain the reverse transcription solution at any suitable temperature and for any of the time periods described herein. For example, in some embodiments, the reverse transcription solution can be maintained at a temperature within a reverse transcription temperature range to produce complementary deoxyribonucleic acid (cDNA) molecules. By rapidly progressing to the reverse transcription, the dwell time during which released RNA are present in the reverse transcription solution can be minimized. Reducing the dwell time can reduce the likelihood that the released RNA will be degraded by ribonuclease (RNase). Limiting such potential degradation by performing the lysing and RT-PCR in a single environment can reduce inconsistencies due to variation in the RNA degradation. Further, the rapid and single-environment methods enabled by the sample preparation module 3200 can allow the RT-PCR methods described herein to be completed without the use of a ribonuclease inhibitor and/or on an unfiltered sample. The reverse transcription temperature range can be, for example, between about 30 C and about 80 C. In other embodiments, the reverse transcription temperature range can be between about 50 C and about 60 C.

In addition to enabling a rapid RT-PCR, the sample preparation module 3200 can also heat the reverse transcription solution to a temperature sufficient to inactivate the one or more lysis or RT enzymes contained therein. For example, the heating element may heat the reverse transcription solution within the channel 3214 to about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C., about 80° C., about 81° C., about 82° C., about 83° C., about 84° C., about 85° C., about 86° C., about 87° C., about 88° C., about 89° C., about 90° C., about 91° C., about 92° C., about 93° C., about 94° C., about 95° C., about 96° C., about 97° C., about 98° C., about 99° C., about 100° C. or greater than 100° C. By heating the reverse transcription solution to a high temperature, the enzymes can be deactivated. In some embodiments, the sample can be heated to about 95 C for about 4 minutes.

As described above, the flow member is in contact with a heating element 3230, which can be, for example, a printed circuit board (PCB) heater. The heating element 3230 includes connectors 3231 and multiple, segmented portions, and thus can independently produce thermal energy into the input reservoir 3211 and the serpentine flow channel 3214. In some embodiments, the heating element 3230 is designed to heat the serpentine portion 3214 of the sample preparation module 3200 while not heating the input reservoir 3211, and vice-versa. Although the heater 3230 is described as including two distinct heating portions, in other embodiments, the heater has only one heating portion, which heats the solution as it flows through the serpentine flow channel 3214 (e.g., in some embodiments, the solution is not actively heated within the input reservoir 3211).

To minimize the heat energy that can inadvertently transfer between the input reservoir 3211 and various portions of the serpentine channel 3214, or even between different portions of the serpentine channel 3214, one or more slots 3232 can be cut in the PCB 3330 to isolate various portions of the heater 3230. For example, in some embodiments, the heater 3230 can include a series of slots and/or openings as described in U.S. Patent Publication No. 2017/0304829 entitled, entitled “Printed Circuit Board Heater for an Amplification Module,” which is incorporated herein by reference in its entirety. Moreover, in some embodiments, the heating element of the heater 3230 is located on an internal layer so the top copper pour (not shown) can be used as a heat spreader to minimize temperature variation along the serpentine path.

The reverse transcription solution, after being flowed through the inactivation process, may be flowed via the output port 3215 through the fluid control valve 3300 and into the inlet port 3217 of the mixing assembly 3250. The mixing assembly 3250 mixes the output from the serpentine flow channel 3214 with the reagents (identified as R3) to conduct a successful amplification reaction. Similarly stated, the mixing assembly 3250 is configured to reconstitute the reagent R3 in a predetermined input volume, while ensuring even local concentrations of reagents R3 in the entirety of the volume. In some embodiments, the mixing assembly 3250 is configured to produce and/or convey a sufficient volume of liquid for the amplification module 3600 to provide sufficient volume output to the detection module 3800. As an example, R3 can include a mixture containing precursors and enzymes used as an ingredient in PCR techniques in molecular biology (in some instances these PCR techniques are applied after the application of RT techniques.) Such mixtures contain a mixture dNTPs (required as a substrate for the building of new DNA strands), MgCl₂, Taq polymerase (an enzyme required to building new DNA strands), a pH buffer and come mixed in nuclease-free water Other compositions and make up can be included.

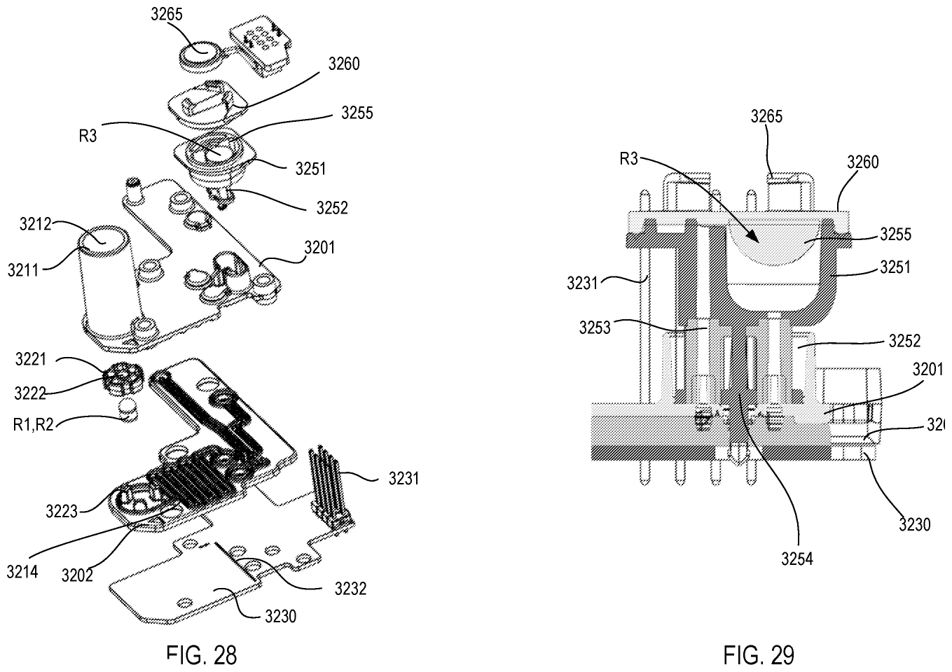

Referring to FIGS. 28 and 29, the mixing assembly 3250 is coupled to the top body 3201 and includes a bottom housing 3251, a top housing 3260, and a vibration motor 3265. The bottom housing 3251 defines a mixing reservoir 3255 and contains the amplification reagents R3 therein. The bottom housing 3251 includes an inlet coupling 3252 and an fluid drive coupling 3253, and is coupled to the top body 3201 by a support member 3254. The top housing 3260 encloses the mixing reservoir 3255 and provides a surface to which the vibration motor 3265 is mounted. The inlet coupling 3252, the fluid drive coupling 3253, and the support member 3254 can be constructed from any suitable material and can have any suitable size. For example, in some embodiments, the inlet coupling 3252, the fluid drive coupling 3253, and the support member 3254 are constructed to limit the amount of vibration energy from the motor 3265 that is transferred into the remaining portions of the sample preparation module 3200. For example, in some embodiments, the inlet coupling 3252, the fluid drive coupling 3253, and/or the support member 3254 can be constructed from a resilient or elastomeric material to allow vibratory movement of the bottom housing 3251 and the top housing 3260 while transferring such energy to the top body 3201.

Although the description above contemplates the reverse transcription reaction being performed within the serpentine channel 314, in other embodiments, the reverse transcription reaction can be performed at different points in the system. For example, in some embodiments, the reverse transcription reaction can be performed in the mixing assembly 3250. In this embodiment, the sample S1 undergoes heat lysis (e.g., within the serpentine channel 3214), then mixed with RT-PCR reagents at the same time in the mixing reservoir 3255. Residual heat of the liquid coming out of the lysis process allows for temperatures that promote the reverse transcription within the mixing assembly 3250. In some embodiments, additional heaters (not shown) can be coupled to the bottom housing 3251, the top housing 3260, or any other suitable structure to promote heating within the mixing reservoir 3255.

After being mixed within the mixing assembly 3250, the prepared sample is then conveyed to the amplification module 3600. The transfer of fluids, including the reverse transcription solution, the reagents or the like is caused by the fluidic drive (or transfer) module 3400. The fluidic drive (or transfer) module 3400 can be a pump or series of pumps configured to produce a pressure differential and/or flow of the solutions within the diagnostic test device 3000. Similarly stated, the fluid drive module 3400 is configured to generate fluid pressure, fluid flow and/or otherwise convey the biological sample and the reagents through the various modules of the device 3000. The fluid drive module 3400 is configured to contact and/or receive the sample flow therein. Thus, in some embodiments, the device 3000 is specifically configured for a single-use to eliminate the likelihood that contamination of the fluid drive module 3400 and/or the sample preparation module 3200 will become contaminated from previous runs, thereby negatively impacting the accuracy of the results. As shown, the fluid drive module 3400 can be a piston pump that is coupled to the reagent module 3700 by one of the clips 3790. The fluid drive module 3400 can be driven by and/or controlled by the electronic control module 3950. For example, in some embodiments, the fluid drive module 3400 can include a DC motors, the position of which can be controlled using rotary encoders (not shown). In other embodiments, the processor 3951 of the electronic control module 3950 can include code to and/or be configured to implement a closed loop method of tracking motor position by monitoring the current draw of motor, as described in International Patent Publication No. WO2016/109691, entitled “Devices and Methods for Molecular Diagnostic Testing.”

The amplification module 3600 includes a flow member 3610, a heater 3630, and a heat sink 3690. The flow member 3610 can be any suitable flow member that defines a volume or a series of volumes within which the that prepared solution S3 can flow and/or be maintained to amplify the target nucleic acid molecules within the solution S3. The heater 3630 can be any suitable heater or group of heaters coupled to the flow member 3610 that can heat the prepared solution within the flow member 3610 to perform any of the amplification operations as described herein. For example, in some embodiments, the amplification module 3600 (or any of the amplification modules described herein) can be similar to the amplification modules shown and described in U.S. Patent Publication No. 2017/0304829 entitled “Printed Circuit Board Heater for an Amplification Module,” which is incorporated herein by reference in its entirety.

In some embodiments, the flow member 3610 defines a single volume within which the prepared solution is maintained and heated to amplify the nucleic acid molecules within the prepared solution. In other embodiments, the flow member 3610 can define a “switchback” or serpentine flow path through which the prepared solution flows. Similarly stated, the flow member 3610 defines a flow path that is curved such that the flow path intersects the heater 3630 at multiple locations. In this manner, the amplification module 3600 can perform a “flow through” amplification reaction where the prepared solution flows through multiple different temperature regions.

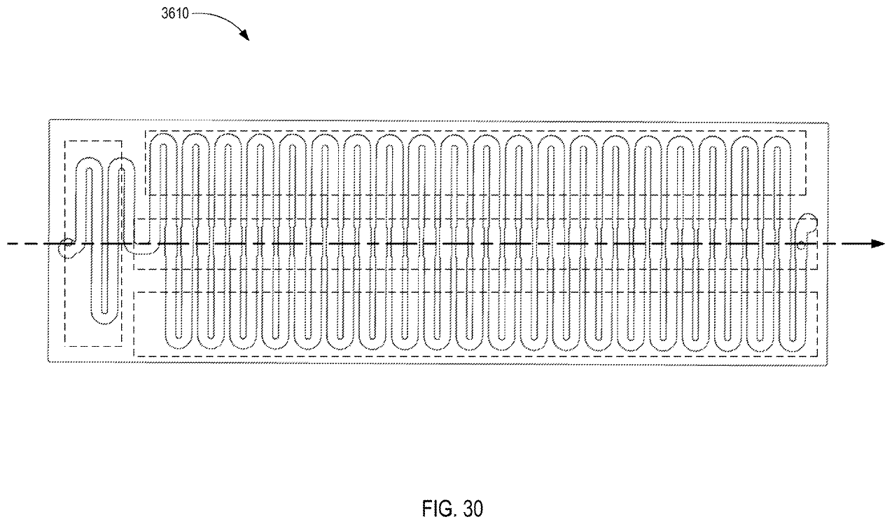

As illustrated in FIG. 30, the flow member 3610 (and any of the flow members described herein) can be constructed from any suitable material and can have any suitable dimensions to facilitate the desired amplification performance for the desired volume of sample. For example, in some embodiments, the amplification module 3600 (and any of the amplification modules described herein) can perform 3000X or greater amplification in a time of less than 15 minutes. For example, in some embodiments, the flow member 3610 (and any of the flow members described herein) is constructed from at least one of a cyclic olefin copolymer or a graphite-based material. Such materials facilitate the desired heat transfer properties into the flow path. Moreover, in some embodiments, the flow member 3610 (and any of the flow members described herein) can have a thickness of less than about 0.5 mm. In some embodiments, the flow member 3610 (and any of the flow members described herein) can have a volume about 150 microliters or greater, and the flow can be such that at least 10 microliters of sample is amplified. In other embodiments, at least 20 microliters of sample are amplified by the methods and devices described herein. In other embodiments, at least 30 microliters of sample are amplified by the methods and devices described herein. In yet other embodiments, at least 50 microliters of sample are amplified by the methods and devices described herein.

The heater 3630 can be any suitable heater or collection of heaters that can perform the functions described herein to amplify the prepared solution. In some embodiments, the heater 3630 can establish multiple temperature zones through which the prepared solution flows and/or can define a desired number of amplification cycles to ensure the desired test sensitivity (e.g., at least 30 cycles, at least 34 cycles, at least 36 cycles, at least 38 cycles, or at least 60 cycles). The heater 3630 (and any of the heaters described herein) can be of any suitable design. For example, in some embodiments, the heater 3630 can be a resistance heater, a thermoelectric device (e.g. a Peltier device), or the like. In some embodiments, the heater 3630 can be one or more linear “strip heaters” arranged such that the flow path crosses the heaters at multiple different points. In other embodiments, the heater 3630 can be one or more curved heaters having a geometry that corresponds to that of the flow member 3610 to produce multiple different temperature zones in the flow path.

Although the amplification module 3600 is generally described as performing a thermal cycling operation on the prepared solution, in other embodiment, the amplification module 3600 can perform any suitable thermal reaction to amplify nucleic acids within the solution. In some embodiments, the amplification module 3600 (and any of the amplification modules described herein) can perform any suitable type of isothermal amplification process, including, for example, Loop Mediated Isothermal Amplification (LAMP), Nucleic Acid Sequence Based Amplification (NASBA), which can be useful to detect target RNA molecules, Strand Displacement Amplification (SDA), Multiple Displacement Amplification (MDA), Ramification Amplification Method (RAM), or any other type of isothermal process.

The detection module 3800 is configured to receive output from the amplification module 3600 and reagents from the reagent module 3700 to produce a colorimetric change to indicate presence or absence of target organism in the initial input solution. The detection module 3800 also produces a colorimetric signal to indicate the general correct operation of the test (positive control and negative control). In some embodiments, color change induced by the reaction is easy to read and binary, with no requirement to interpret shade or hue. The detection module 3800 can be similar to the detection modules shown and described in International Patent Publication No. WO2016/109691, entitled “Devices and Methods for Molecular Diagnostic Testing.”

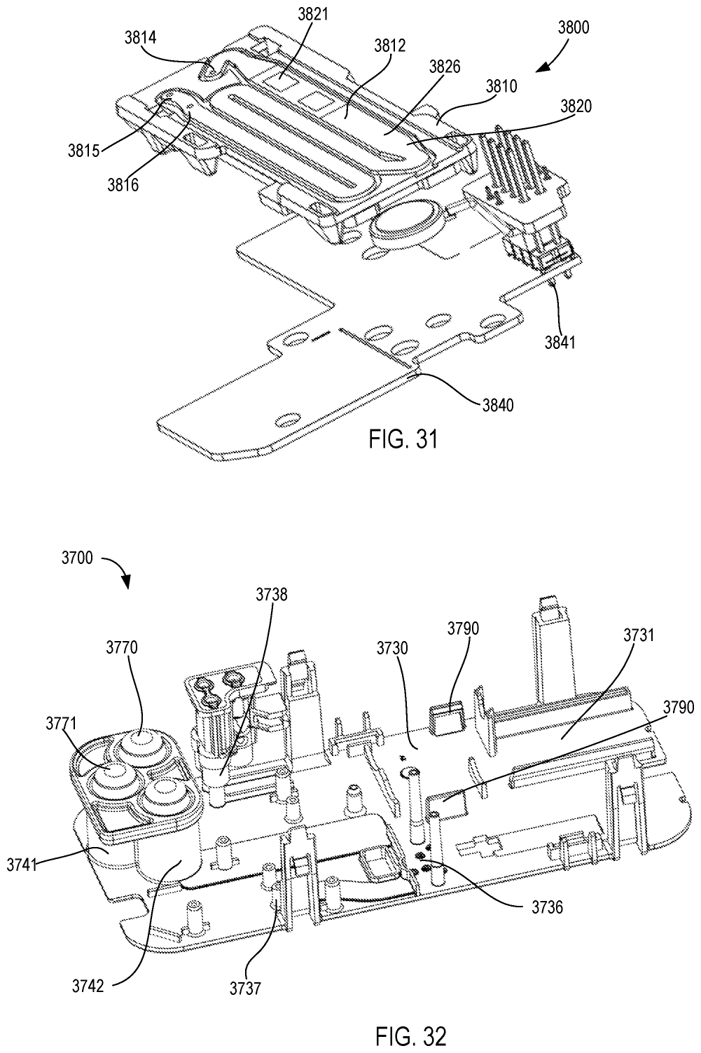

Referring to FIGS. 31 and 32, the detection module includes a lid, a detection housing 3810 and a heater 3840. The heater 3840 can be similar to any of the circuit board heaters described herein and also shown and described in International Patent Publication No. WO2016/109691, entitled “Devices and Methods for Molecular Diagnostic Testing.” The lid and the detection housing 3810 form a flow cell for detection. The housing 3810 defines a detection chamber/channel 3812 having a sample inlet port 3814, a first reagent inlet/outlet port 3815, a second reagent inlet/outlet port 3816. The sample inlet port 3814 is fluidically coupled to the outlet of the amplification module 3600 and receives the amplified sample. The first reagent port 3815 and the second reagent port are coupled to the reagent module 3700 via the fluid interconnect 3738. Thus, in use a wash/blocking reagent (e.g., previously identified as R4) can be conveyed into the detection channel 3812 via the first reagent port 3815 or the second reagent port 3816. Similarly, a detection enzyme (e.g., previously identified as R5) and a detection substrate (e.g., previously identified as R6) can be conveyed into the detection channel 3812 via the first reagent port 3815 or the second reagent port 3816. Additionally, the first reagent port 3815 or the second reagent port 3816 can also be used to receive waste or excess reagents or flows out of the first reagent port 3815 or the second reagent port 3816.

The detection channel 3812 is surrounded or defined by a surface 3820 that includes one or more detection surfaces 3821, as well as non-detection surfaces 3826. The detection surfaces 3821 include a series of capture probes to which the target amplicon can be bound when the detection solution flows across the detection surface 3821. The capture probes can be any suitable probes formulated to capture or bind to the target amplicon. Specifically, in some embodiments, the detection portion 3821 includes five detection surfaces. Each of the detection surfaces are chemically modified to contain a desired capture probe configuration. Specifically, in some embodiments, a first detection surface can include a hybridization probe specific to Neisseria gonorrhea (NG). A second detection surface can include a hybridization probe specific to Chlamydia trachomatis (CT). A third detection surface can include a hybridization probe specific to Trichomonas vaginalis (TV). A fourth detection surface can include non-target probe for a negative control. A fifth detection surface can include a hybridization probe for a positive control (A. fischeri. N. subflava, or the like). In some embodiments, the detection portion 3821 includes three detection surfaces. Each of the detection surfaces are chemically modified to contain a desired capture probe configuration. Specifically, in some embodiments, a first detection surface can include a hybridization probe specific to SARS-COV-2. A second detection surface can include non-target probe for a negative control. A third detection surface can include a hybridization probe for a positive control. The non-detection surfaces 3826 can be those surfaces surrounding the detection surfaces 3821.

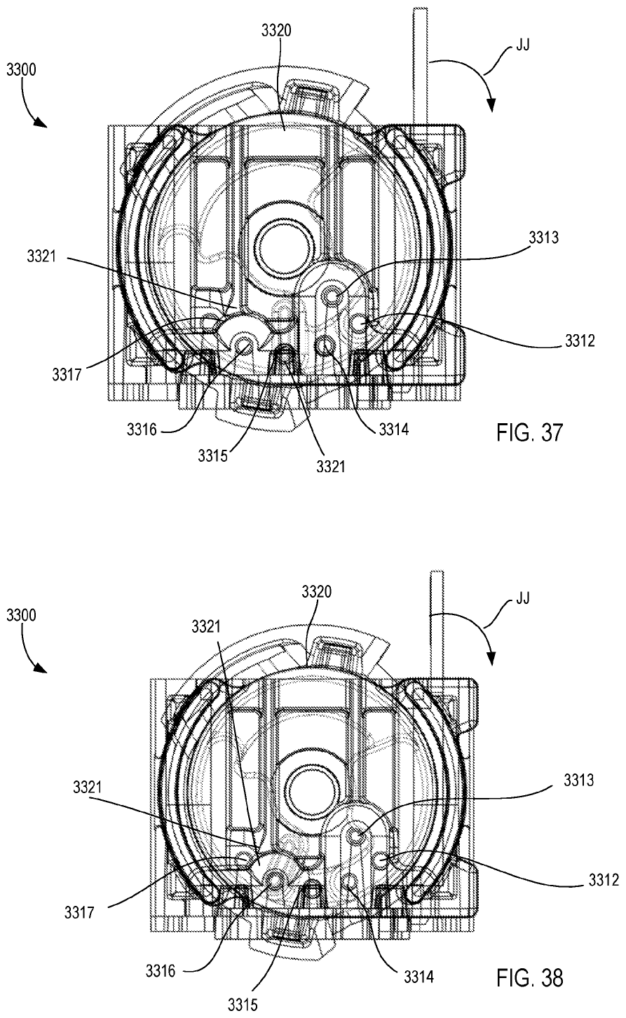

The fluid transfer valve 3300 is shown in FIGS. 7B (schematically) and 34. FIGS. 35-40 show the fluid transfer valve 3300 in several different operational configurations, with the flow (or vent) housing 3310 shown in transparent lines so that the position of the valve disk 3320 can be seen. The fluid transfer valve 3300 includes a flow housing 3310, a valve body (or disk) 3320, a main housing 3330, and a motor 3340. The flow housing 3310 defines a valve pocket within which the valve disk 3320 is rotatably disposed. The flow housing 3310 includes a flow structure that defines at least six transfer (or vent) flow paths, shown in FIGS. 35-40. Specifically, the flow paths include a sample inlet path 3312, a sample outlet path 3313, an amplification path 3314, a wash solution (reagent R4) vent path 3315, a detection enzyme (reagent R5) vent path 3316, and a detection substrate (reagent R6) vent path 3317. The flow housing 3310 includes connection portions where each of the transfer or vent paths can be coupled to the respective modules via the interconnects described herein. Each of the fluid connection/vent ports described above opens into the valve pocket. In this manner, when the valve body 3320 rotates around the center of the valve pocket (as shown by the arrow JJ), the slot channel 3321 of the valve body 3320 can connect various central ports to the other ports depending on their radial and angular position. The use of multiple radii allows not only a single port, but multiple ports at once to be fluidically coupled or vented depending on the configuration.

The valve assembly 3300 can be moved between various different configurations, depending on the angular position of the valve body 3320 within the valve pocket. FIGS. 35-40 show the assembly in various different configurations. FIG. 35 shows the valve assembly 3300 in the home (or initial position), in which the sample inlet path 3312 and the sample outlet path 3313, as well as the other fluid connection/vent ports, are closed. FIG. 36 shows the valve assembly 3300 in a first rotational position, in which the sample inlet path 3312 and the sample outlet path 3313 are opened. With the valve assembly 3300 in the first position, actuation of the fluidic drive module 3400 (in a first direction) can produce a flow of air in a first (or retrograde) direction through the serpentine channel 3214 and into the input reservoir 3211 to facilitate an initial mixing operation, as described above. With the valve assembly 3300 in the first position, actuation of the fluidic drive module 3400 (in a second direction) can produce a flow of the biological sample in a second direction from the input reservoir 3211 into and through the serpentine channel 3214 and then to the mixing assembly 3250. In this manner, the device 3000 can perform the RT-PCR methods as described herein (e.g., the method 50, or any of the other RT-PCR methods). Moreover, the timing of the valve actuation and the power supplied to the fluidic drive module 3400 (e.g., the pump) can be controlled by the electronic control module 3950 to maintain the flow rate through the sample preparation module 3200 (including the serpentine channel 3214) within a range that the desired performance for the RT-PCR can be achieved.

After completion of the mixing process within the mixing assembly 3250, the valve assembly 3300 can be further moved into the second position (not shown). When the valve is in the second position, the amplification path 3314 is opened (i.e., is aligned with the flow slot 3321), thus allowing transfer of the mixed solution (i.e., post RT) to be conveyed into the amplification module 3600. The timing of the valve actuation and the power supplied to the fluidic drive module 3400 (e.g., the pump) can be controlled by the electronic control module 3950 to maintain the flow rate through the amplification module 3600 within a range that the desired performance for the amplification can be achieved. Moreover, with the valve assembly 3300 in the second position, continued actuation of the fluidic drive module 3400 will convey the amplified solution into and through the detection module 3800.

As described herein, the detection operation is accomplished by conveying a series of reagents into the detection module at specific times. Although closing the lid 3050 actuates the reagent module 3700 to open (or release) the reagents from their respective sealed containers, the reagents remain in the reagent module 3700 until needed in the detection module 3800. When a particular reagent is needed, the rotary valve 3300 opens the appropriate vent path (i.e., the wash solution vent path 3315, the detection enzyme vent path 3316, and the detection substrate vent path 3317) to the reagent module 3700. Actuation of the fluidic drive module 3400 applies vacuum to the output port of the reagent module 3700 (via the detection module 3800), thus conveying the selected reagent from the reagent module 3700 into the detection module 3800. FIG. 37 shows the valve assembly 3300 in a third rotational position, in which the detection enzyme vent path 3316 is opened. With the valve assembly 3300 in the third position, actuation of the fluidic drive module 3400 can produce a flow of the detection enzyme (reagent R5) into the detection module 3800. FIG. 38 shows the valve assembly 3300 in a fourth rotational position, in which the wash solution (reagent R4) vent path 3315 is opened. With the valve assembly 3300 in the fourth position, actuation of the fluidic drive module 3400 can produce a flow of the wash (or multi-purpose wash/blocking) solution (reagent R4) into the detection module 3800. FIG. 39 shows the valve assembly 3300 in a fifth rotational position, in which the detection substrate (reagent R6) vent path 3317 is opened. With the valve assembly 3300 in the fourth position, actuation of the fluidic drive module 3400 can produce a flow of the substrate (reagent R6) into the detection module 3800. FIG. 40 shows the valve assembly 3300 in a final position, in which the vent paths are closed.

The device 3000 can be used to perform any of the methods described herein. Referring to FIGS. 41A-41C, to use the device, a biological sample S1 is first placed into the sample input opening 3021 (e.g., using a sample transfer pipette 110), as described above. The lid 3050 is then moved to the closed position, as shown by the arrow KK in FIG. 41B. As described above, closing the lid 3050 encloses the sample input reservoir 3211, actuates the electronic control module 3950 (and/or the processor 3951 included therein), and also actuates the reagent module 3700, as described above. The device 3000 is then plugged in via the power cord 3905 to couple the device 3000 to a power source. In this manner, the device 3000 can, in addition to disposing the sample S1 therein and plugging in the device, be actuated by a single action (i.e., the closing of the lid).

Although the device 3000 is shown and described as producing a visible (or colorimetric) signal that is directly viewed by a user to determine a test result, in other embodiments, a device can include an electronic detection system that produces an electronic signal (e.g., a light output, an audible output, a wireless RF signal, or the like) associated with the test result. For example, FIG. 43 is a schematic illustrations of a molecular diagnostic test device 5000 (also referred to as a “test device” or “device”), according to an embodiment. The test device 5000 is configured to manipulate biological sample to produce one or more output signals associated with a target cell, according to the various systems and the methods described herein. In some embodiments, the test device 5000 can be an integrated device that is suitable for use within a point-of-care setting (e.g., doctor's office, pharmacy or the like), decentralized test facility, or at the user's home. Similarly stated, in some embodiments, the modules of the device, described below, are contained within a single housing such that the test device can be fully operated without any additional instrument, docking station, or the like. Further, in some embodiments, the device 5000 can have a size, shape and/or weight such that the device 5000 can be carried, held, used and/or manipulated in a user's hands (i.e., it can be a “handheld” device). In some embodiments, the test device 5000 can be a self-contained, single-use device.

In some embodiments, the device 5000 (and any of the devices shown and described herein) can be a CLIA-waived device and/or can operate in accordance with methods that are CLIA waived. Similarly stated, in some embodiments, the device 5000 (and any of the other devices shown and described herein) is configured to be operated in a sufficiently simple manner and can produce results with sufficient accuracy to pose a limited likelihood of misuse and/or to pose a limited risk of harm if used improperly. In some embodiments, the device 5000 (and any of the other devices shown and described herein), can be operated by a user with minimal (or no) scientific training, in accordance with methods that require little judgment of the user, and/or in which certain operational steps are easily and/or automatically controlled. In some embodiments, the molecular diagnostic test device 5000 can be configured for long term storage in a manner that poses a limited likelihood of misuse (spoilage of the reagent(s), expiration of the reagents(s), leakage of the reagent(s), or the like). In some embodiments, the molecular diagnostic test device 5000 is configured to be stored for up to about 36 months, up to about 32 months, up to about 26 months, up to about 24 months, up to about 20 months, up to about 18 months, up to 12 months, up to 6 months, or any values there between.

The test device 5000 includes a housing 5001, an actuator 5050, a sample preparation module 5200, a reaction module 5600, a detection surface 5821, and an electronic detection system 5950. In some embodiments, the test device 5000 can include any other components or modules described herein, such as, for example, an amplification module, a detection module, a reagent module that contains on-board reagents (e.g., the reagent module 3700), a rotary valve (e.g., to control flow of reagents and/or sample, such as the valve 3300), or a fluid transfer module (e.g., the fluid drive module 3400). The housing 5001 can be any structure within which the sample preparation module 5200 or other components are contained (or partially contained) to form an integrated device for sample preparation and/or molecular testing. The housing 5001 can be a monolithically constructed housing or can include multiple separately constructed members that are later joined together to form the housing 5001 or be contained therein.

As shown in FIG. 43, the housing 5001 includes an external wall 5010 that defines one or more external features. For example, the housing 5001 defines an input opening 5021 through which a biological sample S1 can be conveyed into the sample preparation module 5200. The housing 5001 also defines a viewing area for seeing a visual display of test results (e.g., via a light output produced by the electronic detection system 5950) or operation. The viewing area can include an opening through the external wall 5010 of the housing 5001. In some embodiments, the viewing area can include a window or clear material through which test results can be viewed. The viewing area can include any suitable features to enhance viewing. For example, in some embodiments, the viewing area includes a beveled edge that surrounds (or partially surrounds) the opening.

The sample preparation module 5200 is configured to manipulate the biological sample S1 for further diagnostic testing. For example, in some embodiments, the sample preparation module 5200 can extract target molecules (e.g., nucleic acid) from the biological sample S1 and can produce an input solution that is conveyed into the reaction module 5600. The sample preparation module 5200 can include any components as described herein to manipulate the biological sample S1 for further diagnostic testing and/or to produce a solution for detection of a target molecule (e.g., nucleic acid). For example, in some embodiments, the sample preparation module 5200) can include one or more heaters, one or more chambers within which the biological sample S1 can be manipulated, one or more mixing reservoirs, and/or certain on-board reagents (e.g., a lysing buffer, an RT enzyme, a control substance, or the like). In some embodiments, the sample preparation module 5200 can function merely as a sample holding or mixing reservoir. For example, in some embodiments, the sample preparation module 5200 can contain the desired amplification reagents to facilitate a desired amplification according to any of the methods described herein. In other embodiments, the sample preparation module 5200 is configured to extract nucleic acid molecules from the biological sample S1 and can produce an input solution S2 (see FIG. 1) that is conveyed into the reaction module 5600.

The reaction module 5600 defines an internal reservoir (e.g., a reaction chamber or reaction volume) that receives the input solution from the sample preparation module 5200 and amplifies a target molecule therein to produce an output containing a target amplicon. The reaction volume can be formed from any suitable structure that defines a volume or a series of volumes within which the input solution S2 can flow and/or be reacted to produce a solution for subsequent detection. Thus, the reaction module 5600 can function as an amplification module, a lysis module, or any other module within which a reaction can occur to facilitate detection of the target polynucleotide sequence. In some embodiments, the reaction module 5600 can amplify the target nucleic acid molecules therein to produce an output detection solution that contains a target amplicon (or multiple target amplicons) to be detected. In accordance with some embodiments, the reaction module can include one or more components of an amplification modules and/or one or more components of a detection module. For example, the reaction module 5600 can include a heater that can heat the input solution to perform any of the amplification operations as described herein.

Although the amplification modules described herein are generally described as performing a thermal cycling operation on the input solution S2, in other embodiments, the reaction module 5600 (and any of the amplification modules described herein) can perform any suitable thermal reaction to amplify nucleic acids within the solution. In some embodiments, the reaction module 5600 (and any of the amplification modules described herein) can perform any suitable type of isothermal amplification process, including, for example, Loop Mediated Isothermal Amplification (LAMP), Nucleic Acid Sequence Based Amplification (NASBA), which can be useful to detect target RNA molecules, Strand Displacement Amplification (SDA), Multiple Displacement Amplification (MDA), Ramification Amplification Method (RAM), or any other type of isothermal process.

In accordance with some embodiments, the reaction module 5600 is configured to react the biological sample (identified as the processed solution S3 below) with one or more reagents to cause production of one or more assay signals to indicate presence of the target polynucleotide sequence. Although the biological sample is identified as a portion (i.e., S3) of the initial biological sample (i.e., S1) that has been processed, reacted or prepared within the sample preparation module 5200 and the reaction module 5600, in other embodiments, the portion of the biological sample that is further reacted within the reaction module 5600 can be any suitable portion of the initial biological sample S1. As described herein, the presence of the target polynucleotide sequence can indicate the presence of a target organism, whether the target organism is susceptible to a course of treatment, whether the target organism is resistant to a course of treatment, or other characteristics of the target organism. The reaction module 5600 defines a detection volume within which the biological sample and one or more reagents (see reagent R discussed below) are reacted. The reacting can be performed by combining (e.g., mixing) the reagent R and the biological sample S3 within the reaction module 5600, by introducing each of the reagent R and the biological sample S3 into the reaction module 5600 (either at the same time or in a sequential manner), by conveying the biological sample S3 into the reaction module 5600, within which the reagent R has been stored for use, or any other suitable method for producing the desired reaction. In some embodiments, the reaction module 5600 can include one or more detection surfaces to which one or more probes are attached. As described herein, such probes can be designed to permit annealing or hybridization of a target amplicon with sufficient specificity to permit detection of the presence (or absence) of a target amplicon indicating the presence of the target polynucleotide sequence. In other embodiments, the detection module can include one or more detection chambers in which different reagents or probes can be combined or reacted with the biological sample to produce a series of assay signals.

The detection surface 5821 is a surface from which the target amplicon is detectable by producing a visible signal. Specifically, as described herein, the output solution from (or within) the amplification module 5600 can be reacted with one or more reagents to produce a signal (or output) to indicate presence or absence of a target organism in the biological sample S1. In some embodiments, the detection surface 5821 can retain the target amplicon produced during amplification within a single region to produce a visible signal. In some embodiments, the detection surface 5821 can include a series of capture probes to which the target amplicon can be bound when the output solution is in contact with the detection surface 5821. The capture probes can be any suitable probe of the types described herein formulated to capture or bind to the target amplicon. In some embodiments, the detection surface 5821 is within the reaction module 5600. For example, in some embodiments, a portion of a wall that defines the reaction volume can also include the detection surface 5821. In this manner, the amplification reaction and reaction to produce the signal can occur within the same module (i.e., the reaction module 5600. In other embodiments, the detection surface 5821 can be spaced apart from (or outside of) the reaction module 5600. For example, in some embodiments the device 5000 can include a separate detection module (not shown) that defines a detection channel and includes the detection surface 5821 within the detection channel. The detection channel is in (or can be placed in) fluid communication with the reaction module 5600. In this manner, the output solution containing the target amplicon can be conveyed into the detection channel and across the detection surface 5821.

The electronic detection system 5950 includes a digital read module 5951 that is configured to receive a signal (and determine, based on the signal, whether a color signal from the corresponding detection surface is present). This system allows for reading of the detection surface internal to the device 5000 without the need for an external window or direct viewing of the detection surface 5821. Further examples of an electronic detection system are disclosed in International Patent Publication No. WO2020/223257A1 entitled “Molecular Diagnostic Devices with Digital Detection Capability and Wireless Connectivity,” herein incorporated by reference in its entirety

As shown in FIG. 43, one or more of the modules or components of the test device 5000 are biased against one or more other surfaces. In some embodiments, the other surfaces can be components other than the housing. For example, the detection surface 5821 is biased against an electronic detection system 5950. In this manner, the distance between the detection surface 5821 and any sensors (e.g., photodiodes) of the electronic detection system 5950 can be maintained at a substantially constant value regardless of part-to-part variations and manufacturing tolerance. The detection surface 5821 (or a detection module) can also be maintained in contact the electronic detection system 5950 to minimize light leakage that can cause undesirable variation in the output of the electronic detection system 5950. For example, in some embodiments, the device can include a mask or light-sealing gasket that surrounds the detection surface 5821 and is between the detection surface 5821 and the electronic detection system 5950. Such a gasket can limit the entry of stray light into and to contain any light produced by the electronic detection system 5950 within the desired detection region. Biasing the detection surface 5821 against the electronic detection system 5950 can provide a seal region to limit light transmission into or out of the detection region. In some embodiments, the device can operate without a gasket. The biasing force can be sufficient to limit light leakage in the absence of a gasket or other sealing device. By limiting light leakage, the test results can be more easily determined by the detection system. In some embodiments, the biasing force is sufficient to create a liquid seal between the detection surface 5821 and the electronic detection system 5950. Such a liquid seal can protect components from outside liquids or contain liquids within the reaction module 5600. In some embodiments, the biasing force can supplement the operation of a gasket between the detection surface 5821 and the detection system 5950.

While the electronic detection system 5950 can be biased against the external wall 5010 of the housing in some embodiments, in other embodiments, the electronic detection system 5950 is set apart from the housing with the detection surface 5821 biased against the electronic detection system 5950 and not the housing. In some embodiments, one of the modules within the housing 5001 includes (or be supported by) a support substrate 5100. The support substrate 5100 engages one or more biasing members 5110 such that the support substrate 5100 is biased in one direction in the housing. In this example, the support substrate 5100 is biased towards the electronic detection system 5950. Thus, the support substrate 5100 can apply a biasing force F, via the biasing members 5110, such that components of one or more modules are pressed against the electronic detection system 5950. Specially, the biasing force causes the detection surface to be biased against the electronic detection system 5950. In this manner, the biasing force (or preload) maximizes the engagement between the detection surface 5821 and the electronic detection system 5950 by ensuring that the respective surfaces are as close to one another as possible. In situations in which the detection surface is spaced apart from of the electronic detection system 5950, the ability of the electronic detection system 5950 to read the detection surface 5821 may be affected.

The support substrate 5100 can be any suitable structure within the housing 5001. In some examples, the support substrate 5100 can form a component of any of the modules described herein (e.g., a flow manifold 3035 discussed below). In other examples, the support substrate 5100 can be a separate component (e.g., a supporting structural component) that extends between different modules or supports a single module. As an example, FIG. 43 illustrates the support substrate 5100 supporting both the 5200 and the reaction module 5600 (as well as the detection surface 5821). In other examples, however, the support substrate 5100 can support just the sample preparation module 5200. Alternatively, the support substrate 5100 can support just the reaction module 5600. Additionally or alternately, the support substrate 5100 can support other modules.

In some examples, the biasing members can include one or more springs (e.g., compression, extension, torsion, constant force, Belleville, drawbar, volute, flat, gas, etc.). In some examples, the biasing members 5110 can be integral to one or more components of the device 5000. The biasing members 5110 includes any suitable structure that applies a biasing force between two components such as, the housing, a module, and/or the support substrate. In such examples, the biasing members 5110 can be a molded flexible protrusion from the support substrate such as tabs. Moreover, although the device 5000 is shown as including multiple biasing member, in other embodiments, a device can include only a single biasing member.

In some embodiments, the housing 5001 (or any other suitable structure within the device 5000) can include any suitable structure (e.g., shoulders, mounts, etc.) to apply a counterforce against the biasing members 5110. In such embodiments, the mounts can form an attachment point for the biasing members 5110 or the mounts can function as ground point for the biasing members. The mounts can be any suitable feature for locating, retaining, anchoring, or otherwise supporting feature for the biasing members.

In accordance with some embodiments, the biasing members 5110 produce a biasing range or distance (T1 minus T2). The biasing range is the unencumbered travel of the biasing mechanism in relationship to the stack up height T1 of the supported modules and/or components. Thus, the biasing range is the range of travel for the biasing mechanism that allows the support substrate to move to a position, when not restricted by supported modules or components, that is at a substrate distance T2 from the opposing interior housing surface. In some embodiments, the stack up height T1 is greater than the substrate distance T2. In this way, the biasing members 5110 are able to maintain a bias of the highest component in the module/component stack against the electronic detection system 5950 creating a component preload. In the example illustrated in FIG. 43, the detection surface 5821 is preloaded against the electronic detection system 5950 forming a contact region between the two.

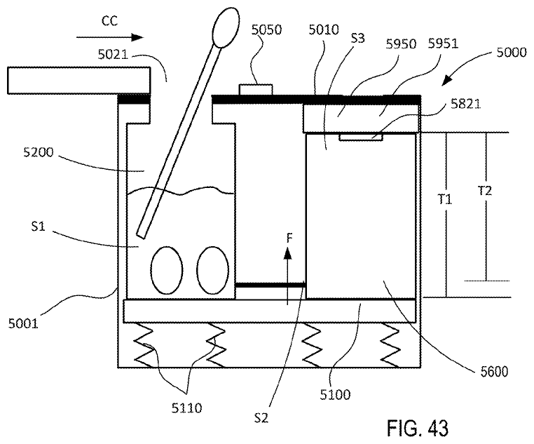

FIGS. 44-50 are ornamental views of the molecular diagnostic test device in accordance with one embodiment.

In some embodiments, a molecular diagnostic test device can be configured to use a reduced amount of PCR reagent than would conventionally be required for the amount of biological sample input into the device. In this manner, the cost of the device can be reduced and valuable resources can be conserved. Specifically, in some embodiments, a molecular diagnostic test device includes a housing, a sample preparation module within the housing, and a reaction module disposed within the housing. The sample preparation module defines an input reservoir configured to receive a sample quantity of a biological sample containing a target molecule. The input reservoir is in fluid communication with a mixing reservoir containing a quantity of a PCR reagent. The ratio of the sample quantity to the reagent quantity is between a 3:1 ratio and a 7:1 ratio. In some embodiments, the ratio of sample quantity to reagent quantity can be 5:1. For example, there can be 500-700 μL of sample quantity and 100-200 μL of reagent quantity. The sample preparation module is configured to produce an amplification solution containing a portion of the biological sample and the PCR reagent. The reaction module is configured to receive the amplification solution containing the target molecule and the PCR reagent and amplify the target molecule to produce an output containing a target amplicon. The molecular diagnostic test device is configured to produce a signal in response to a presence of the target amplicon being detected within the amplification solution.

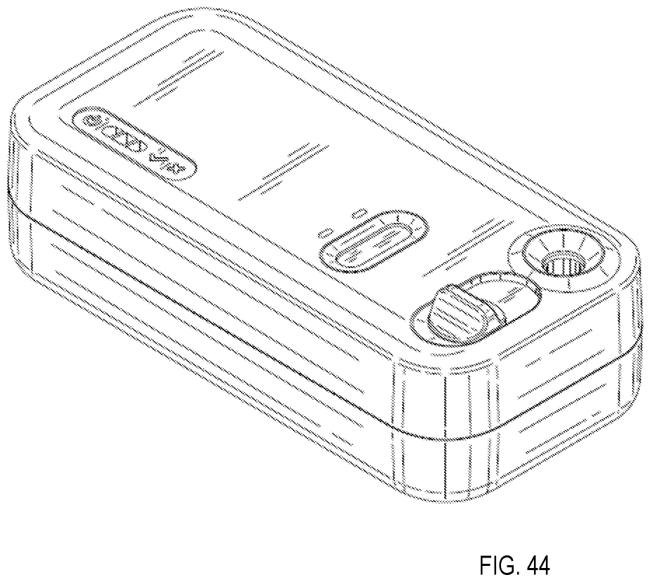

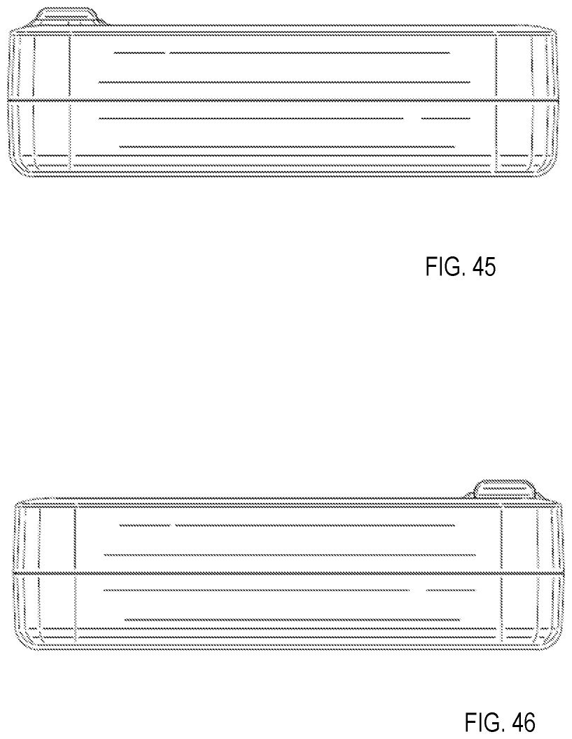

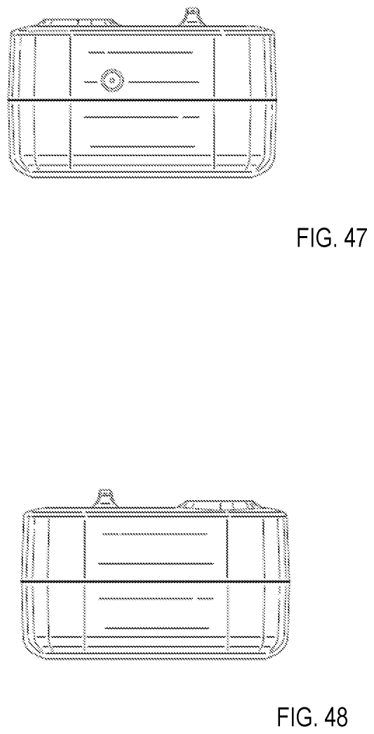

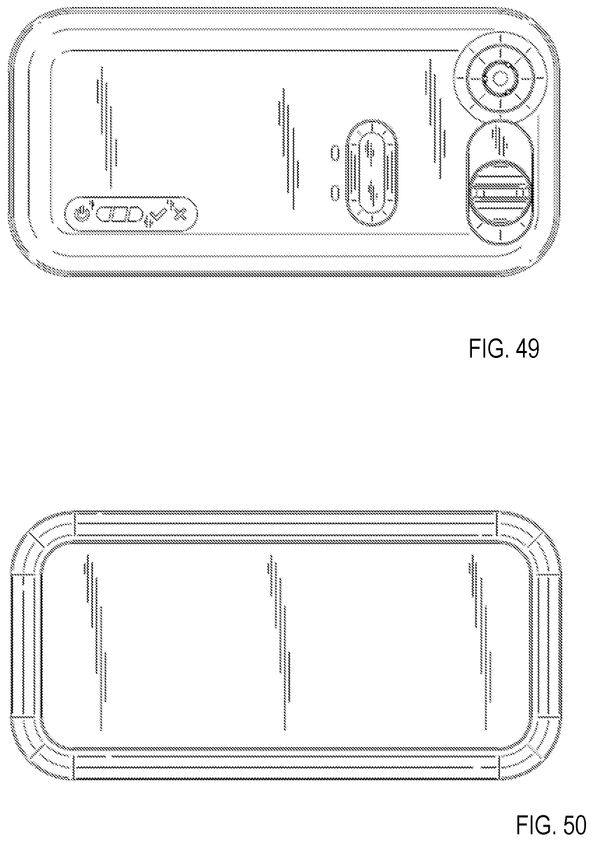

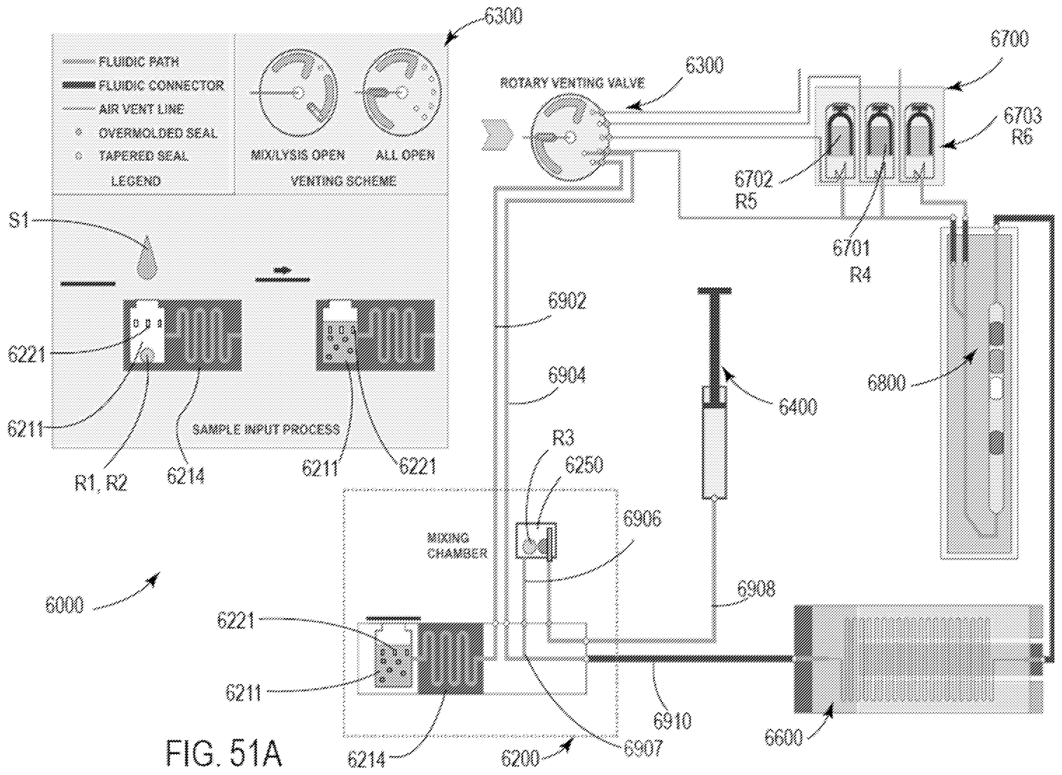

In some embodiments, any of the molecular diagnostic test devices described herein can be configured to include the ratio of sample quantity to reagent quantity in a manner that reduces reagent use. For example, FIGS. 51A-51E are schematic illustrations of various portions the processes operating within a molecular diagnostic test device 6000 in accordance with some embodiments. Like the other embodiments discussed herein, the test device 6000 is configured to manipulate a biological sample S1 to produce one or more output signals associated with a target cell. Specifically, the device 6000 includes a sample preparation module 6200, a fluidic drive (or fluid transfer) module 6400, a valve 6300, an amplification module 6600, and detection module 6800. While the amplification module and the detection module are shown by way of example as two different modules, it is also appreciated that both operations can be accomplished in a reaction module as discussed above, which can provide amplification of the amplicon and detect the target molecule. Reagents in 6700 can be used for the detection. The molecular diagnostic test device 6000 can also include a control module (not shown). Although the schematic illustrations in FIGS. 51A-51E show the device 6000 as including a separate amplification module and detection module, in other embodiments, amplification and detection can be performed in the same module such as the reaction module discussed above. Further, in some embodiments, the device 6000 need not include the flow channel 6214 or perform any reverse transcription reactions.

Each of the FIGS. 51A-51E illustrated different portions of a process for circulating one or more solutions through the molecular diagnostic test device 6000. The embodiments discussed herein are applicable to the other devices, systems and methods discussed above with reference to the other embodiments, such as molecular diagnostic test device 1000, 2000, 3000 etc. In particular, the system and processes described with reference to the molecular diagnostic test device 6000 illustrated in FIGS. 51A-51E reduce costs by reducing the volume of PCR reagents including enzymes, primers, dNTPs, salts, and other suitable components used in the molecular diagnostic test device 6000, while still producing a sufficient volume of the biological sample to provide sufficient volume to the detection module 6800 to facilitate the desired multiplex assay. The molecular diagnostic test device 6000 reduces the enzyme use without introducing measurement error or decreased sensitivity or specificity due to low concentrations in the reaction or inconsistent concentrations. Specifically, the molecular diagnostic test device 6000 is configured to receive a sufficient volume of the biological sample to provide sufficient volume to the detection module 6800 to facilitate the desired multiplex assay. But, because of the system volumes within the flow paths of the device, only a portion of the sample volume is output to the detection module. Accordingly, in some embodiments, a device can be configured to only mix the portion of the biological sample that will be conveyed into the detection module (also referred to as the amplification solution or detection solution). The remaining portion (also referred to as the drive solution) can remain unmixed (or mixed with a lower amount of the PCR reagent) and can be used to drive (i.e., form a hydraulic link with) the amplification solution.

For example, in some embodiments, the molecular diagnostic test device 6000 can receive a volume of 500 μL-700 μL of the biological sample S1 solution. Less than that amount is used to mix with PCR reagent and even less is used for the detection of pathogens within the detection module 6800. For example, 250-350 μL of the of the sample is mixed with the PCR reagents (e.g., R3). In this example, 70-300 μL of the mixed sample and reagent is then used in the detection process. The remaining solution (e.g., 250-350 μL of solution) is used to maintain primed fluidic pathways. Stated another way the remaining solution (e.g., the 250-350 μL of solution that is not mixed with the reagent) drives the solution that used for detection. Said yet another way, the remaining solution forms a hydraulic link between the pump and the portion of the solution used for detection. In some embodiments, the biological sample S1 solution is about 650 μL: half of that is mixed with PCR reagent; and then the portion of the mixed solution that moves on to detection is about 100 μL. The remaining solution (e.g., about 350 μL) can be utilized as the drive solution suited for its bulk volume to keep fluidic pathways primed (i.e., substantially devoid of gas). The limits of detection (LoD) of molecular diagnostic test device 6000 based on the volume of amplicon driven into detection module 6800. The remaining solution (drive solution) can account for using between 25 percent and 75 percent of the PCR enzyme, thus increasing the cost. However, limiting the enzymes to reacting with the amplification solution but not the drive solution limits these costs. The drive solution, however, allows the pathways (e.g., 6902, 6904, 6906, 6908, 6910) to remain primed (i.e., substantially devoid of gas) for fluidic control while minimizing or eliminating reacting the drive solution with the enzyme. Having a compressible gas within the flow paths can disrupt the flow of the amplification solution within the amplification module and can otherwise produce inconsistent amplification performance. Moreover, gas bubbles within the detection module 6800 can reduce detection accuracy. Thus, the drive solution can maintain a hydraulic link to limit (or substantially eliminate) having gas (e.g., air) within the flow paths.

Similar to other embodiments described herein, the sample preparation module 6200 includes a top body, a bottom body, a heater, and a mixing assembly 6250. The top body and the bottom body can be referred to collectively as a sample preparation housing, a flow member or a reverse transcription chamber. The flow member can be constructed from multiple pieces that are coupled together or, in other embodiments, the flow member can be monolithically constructed. The sample preparation housing defines a sample input opening, a input reservoir 6211, and a serpentine flow channel 6214. In some embodiments, the top body and/or the bottom body can define one or more vents. Such vents can allow air to flow into or out of the sample preparation module 6200 (e.g., the input reservoir 6211, mixing assembly 6250, the serpentine flow channel 6214, etc.) as sample is conveyed into and/or out of the sample preparation module 6200. Additionally, the sample preparation module 6200 can include a set of fluid interconnects that allow for fluidic coupling of the sample preparation module 6200 to the fluid transfer valve 6300 and other components within the device 6000. Similar to embodiments discussed above, the sample preparation module 6200 can include the input reservoir 6211 which can include one or more retention screen 6221 is positioned to separate the input reservoir 6211 into a first portion and a second portion.

The solution from the input reservoir 6211 can be flowed through the fluid control valve 6300 and into an inlet of the mixing assembly 6250. The mixing assembly 6250 mixes the output conveyed from the input reservoir and, in some embodiments, the serpentine flow channel 6214, with the reagents (identified as R3) to conduct a successful amplification reaction. Similarly stated, the mixing assembly 6250 is configured to reconstitute the reagent R3 in a predetermined volume of the biological sample, while ensuring even local concentrations of reagents R3 in the entirety of the volume. In some embodiments, the mixing assembly 6250 is configured to produce and/or convey a sufficient volume of liquid for amplification in the reaction module 6600 to provide sufficient volume output for detection module 6800.

In accordance with some embodiments, the molecular diagnostic test device 6000 includes one or more of flow channels or interconnects 6902, 6904, 6906, 6908, 6910. Via interaction between the fluidic drive (or fluid transfer) module 6400, valve 6300 and one or more vents (e.g., such as those illustrated with valve 6300 in FIGS. 51A-51E) the one or more of flow channels 6902, 6904, 6906, 6908, 6910 route the solutions throughout the device 6000. The flow channels or interconnects 6902, 6904, 6906, 6908, 6910 can be separate tubes or alternatively can be bores within various components within the device. The flow channels can include the flow members discussed above with regard to other embodiments and FIGS. In some embodiments, flow channel 6902 can fluidically connect the input reservoir 6211 and/or the serpentine flow channel 6214 with the valve 6300. Referring to FIG. 51A, in some embodiments, the flow channel 6904 can fluidically connect the valve 6300 with one or more of the mixing reservoir 6250, the reaction module 6600, or a junction 6907 between a flow channel 6906 to the mixing reservoir and flow channel 6910 to the reaction module 6600. In some embodiments, the flow channel 6906 can fluidically connect the junction 6907 to the mixing reservoir 6250. In some embodiments, the flow channel 6910 can fluidically connect the junction 6907 to the reaction module 6600. In some embodiments, the flow channel 6908 can fluidically connect the junction 6907 to the reaction module 6600 can fluidically connect the mixing reservoir 6250 to the fluidic drive (or fluid transfer module) 6400.

In some embodiments, the control system for the device 6000 is configured to operate the valve 6300 such that less than all of the biological sample S1 (and in some embodiments any reagents R1, R2 mixed therewith in an input reservoir 6211 forming the input solution) is directed to the mixing reservoir 6250 and mixed with the reagent R3. Specifically, the input reservoir 6211 can be sized to receive a sufficient volume of the biological sample to provide sufficient volume to the detection module 6800 to facilitate the desired multiplex assay. For example, in some embodiments, the input reservoir 6211 can receive between about 500-700 μL of the biological sample. In some examples, the input reservoir 6211 is sized to receive about 650 μL of biological sample S1. The valve can be configured to allow the fluidic drive 6400 to draw an input solution quantity into the mixing reservoir 6250. In some examples, the input solution quantity that is drawn into the mixing reservoir 6250 is approximately equal to the volume of the fluid pathways used during detection (e.g., the volume of the detection module 6800 plus any additional solution volume downstream of the detection module 6800). In some examples, as illustrated in FIG. 51B, the fluidic drive 6400 draws in 50-150 μL. In some examples, as illustrated in FIG. 51B, the fluidic drive 6400 draws in about 100 μL of the input solution into the mixing reservoir. As shown in FIG. 51B, the channels 6902, 6904, and 6906 are primed with fluid as shown by greyscale blocking over these channels in FIG. 51B. The input solution is mixed with reagent R3 in the mixing reservoir 6250 forming the amplification solution. The quantity of the reagent R3 stored within the mixing reservoir is such that most or all of the reagent is combined with the input solution to form the amplification solution during this step. For example, 30-100 units of the reagent R3 are stored in the mixing reservoir. In a more specific example, about 40 units are stored in the mixing reservoir. Units are the metric used in the industrial standard for quantity comparisons of a specific reagent. This reagent is then mixed substantially fully with the first batch of input solution, forming what is referred to herein as the amplification solution. The mixing can be performed by any suitable methods as described herein, such as, for example by vibration of the mixing reservoir 6250, by injecting air into the mixing reservoir, or any other methods as described herein. In some embodiments, the device 6000 or more particularly the sample preparation module is filterless. Both filtered and filterless systems are contemplated herein although the figures illustrate filterless systems.

In FIG. 51C, the amplification solution is then driven out of the mixing reservoir 6250 filling flow channel 6910 and the junction 6907. Some of or all of flow channel 6906 can also be filled during this operation. Additionally, as shown, the input solution is maintained in flow channels 6904 and 6902 during this operation. For example, a vent at the valve 6300 can be closed. This vent is fluidically in line with one or both of flow channels 6902 and 6904. Additionally, a vent that is fluidically in line with flow channels 6906 and 6910 (e.g., a vent that is connected with the valve 6300 passed the detection module 6800 or reagents 6700) can be opened. This operation allows for the amplification solution to be driven into flow channels 6906 and 6910 without moving input solution in flow channels 6902 and 6904.

In FIG. 51D, the input solution is driven into the mixing reservoir 6250 filling flow channel 6906 and the junction 6907. Enough input solution is driven into the mixing reservoir 6250 to drive the amplification solution through the reaction module 6600 and to detection 6800. In some embodiments, flow channel 6906 can also be filled. Input solution is held in flow channels 6904 and 6902. Opening a vent at the valve 6300 that is fluidically in line with one or both of flow channels 6902 and 6904 while also closing a vent that is fluidically in line with flow channel 6910 (e.g., a vent that is connected with the valve 6300 passed the detection 6800 or reagents 6700) allows for the input solution to be driven into flow channel 6906 (e.g., with the fluid drive 6400) and the mixing reservoir 6250 without moving the amplification solution in flow channel 6910.

In FIG. 51E, the input solution is driven out of the mixing reservoir 6250 thereby forming a drive solution that drives the amplification solution. The drive solution continues to drive the amplification solution into the reaction module 6600 allowing amplification of the target amplicon to occur. The drive solution then drives the amplification solution into the fluid pathways used for detection within the detection module 6800. In some examples, the amplification solution and the fluid pathways used for detection are approximately the same volume. Additional amplification solution can be produced to account for tolerances and/or clearing bubbles.

While described above in the examples in terms of ratios, concentrations, units etc., it should be appreciated that in some embodiments, a drive solution (e.g., input solution with less reagent than the amplification solution) drives the amplification solution (e.g., input solution with sufficient reagent for amplification of the amplicon and detection of the same). Efficiencies are found in the system when a portion of the input solution is used to drive amplification solution without mixing it with reagent, thereby conserving the reagent.

In some embodiments, the flow channel 6910 (the portion of flow channel extending between junction 6907 and the reaction module 6600) is equal to or greater than the volume of flow channel used for detection such as the flow member within the detection module 6800 (see FIG. 51E with the flow channel used for detection blocked out in grey scale blocks indicated presence of the amplification solution). Preferably, any excess in volume of the flow channel 6910 is minimized to accommodate tolerances, bubble clearance, or other incidental waste, while still providing sufficient amplification solution for operable detection.

In some embodiments, the amplification solution can be conveyed back into the flow channel 6906 and into the mixing reservoir 6250 to clear any bubbles that become trapped at junction 6907. In such embodiments, the solution that clears the bubbles between junction 6907 and flow channel 6906 is withdrawn back into the mixing reservoir.

The reagent module 6700 includes a reagent manifold (or housing) 6730, three reagent containers 6701, 6702, 6703, and a deformable support member 6770 (see FIGS. 51A-51E). The reagent module 6700 provides mechanisms for long-term storage of reagents within the sealed reagent containers, actuation of the reagent containers to release the reagents from the reagent containers for use during the methods described herein. In addition to providing storage and actuating functions, the reagent module 6700 also provides fluid interconnections to allow the reagents and/or other fluids to be conveyed within the device 6000. Specifically, as described herein, the reagent module 6700 is fluidically coupled to the fluid transfer valve 6300 in a manner that allows selective venting, fluid coupling, and/or conveyance of the reagents and substances within the device 6000. The reagent module 6700 stores packaged reagents. The reagents can include any one or more of reagent R4 (a dual-purpose blocking and wash solution), reagent R5 (an enzyme reagent), and reagent R6 (a substrate), and allows for easy un-packaging and use of these reagents during detection.

While some embodiments of device 6000 are discussed herein, some portions of the device discussion are omitted for clarity. It will be appreciated that the various components discussed above with regards devices 1000, 2000, and 3000 are applicable to the device 6000 and contemplated herein. For example, components such as housings, structural features, modules, fluid sources, reagents, detection components, reservoirs and other suitable components are applicable to device 6000.

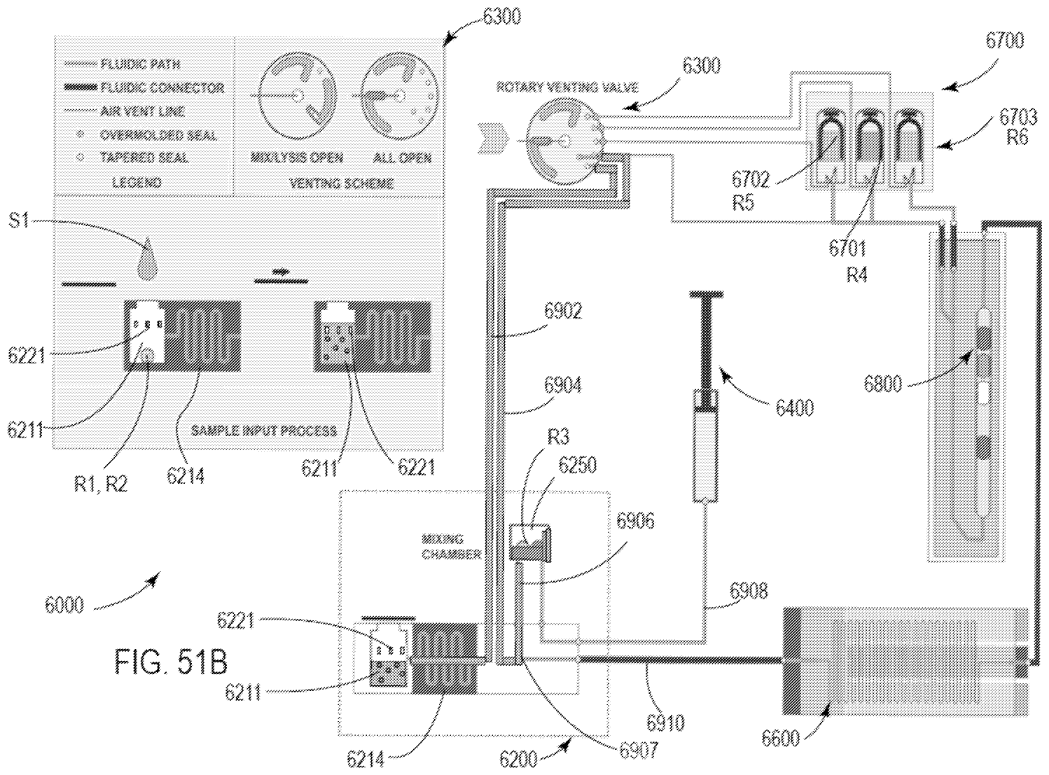

FIG. 52 is a flow chart illustrating a method 7000 for detecting a nucleic acid using a molecular diagnostic test device. The method 7000 can be performed on any of the devices described herein, included the device 6000. The method includes conveying a biological sample into a sample preparation module within the molecular diagnostic test device via an input opening at 7002. The method also includes actuating the molecular diagnostic test device at 7004. The actuation causes the molecular diagnostic test device to mix, within the sample preparation module, the biological sample with a first reagent to produce an input solution at 7006. The actuation causes the molecular diagnostic test device to convey, via a pump, a first portion of the input solution into a reservoir that contains a PCR reagent to form a amplification solution at 7008. The actuation causes the molecular diagnostic test device to convey, via the pump, the amplification solution out of the reservoir and into a flow path between the sample preparation module and a reaction module within the molecular diagnostic test device at 7010. The actuation causes the molecular diagnostic test device to convey, via the pump, a second portion of the input solution into the reservoir at 7012. The actuation causes the molecular diagnostic test device to convey the amplification solution into the reaction module via the pump by driving the second portion of the input solution with the pump, the second portion being positioned between pump and the amplification solution such that the second portion of the input solution exerts a drive force on the amplification solution at 7014. The actuation can cause the molecular diagnostic test device to heat the amplification solution within the reaction volume thereby producing an output solution containing a target amplicon. The actuation causes the molecular diagnostic test device to react within the molecular diagnostic test device each of (i) the output solution and (ii) a reagent formulated to produce a signal that indicates a presence of the target amplicon within the output solution at 7018. The method also includes reading a result associated with the signal at 7020.

As discussed above, the constituents within the input reservoir (e.g., the input reservoir 6211) can be mixed via a forced fluid into the reservoir. Additionally, or alternatively, the mixing reservoir (e.g., 6250) can likewise be mixed via a force fluid into the reservoir. Accordingly, the mixing reservoir can operate without another mixing apparatus (e.g., a mixing motor, vibrator motor, etc.) For example, in some embodiments, the valve assembly 6300 can be moved between various different configurations to allow flow of fluids within the mixing reservoir 6250. In some embodiments, the valve assembly 6300 can direct a stored air supply (e.g., stored in the fluid transfer module 6400) into the mixing reservoir 6250 to cause bubbling in the mixing reservoir 6250 to mix the input solution and the solid reagent R3. In accordance with some embodiments, the fluid source is the fluidic drive 6400. In one example, the fluidic drive includes a fluid pump with an air reservoir within the fluid pump. The fluid pump is in fluid communication with the mixing reservoir 6250 such that air from the air reservoir can be pumped to the reservoir. When the air forms bubbles in the reservoir the rising bubbles agitate the input solution and the reagent. As shown in FIGS. 51A-E, the plunger on the pump can be drawn back pulling fluid in towards the pump. A vent is opened via valve 6300 allowing air to be pulled up and through the bottom of reservoir 6250.

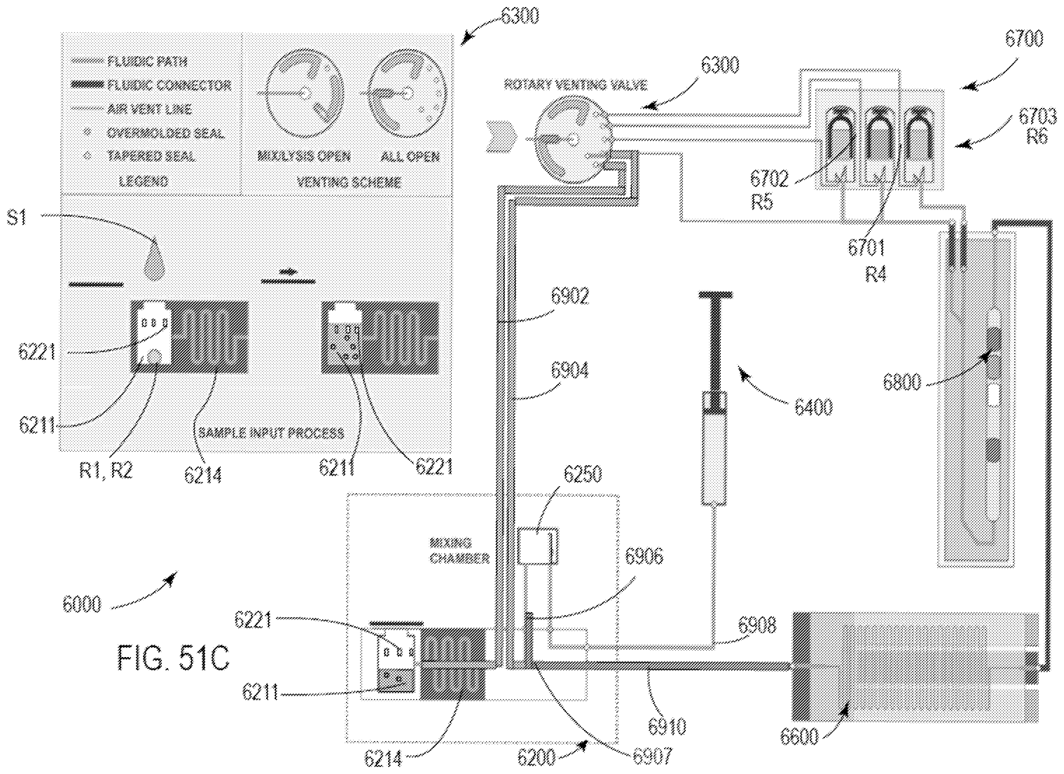

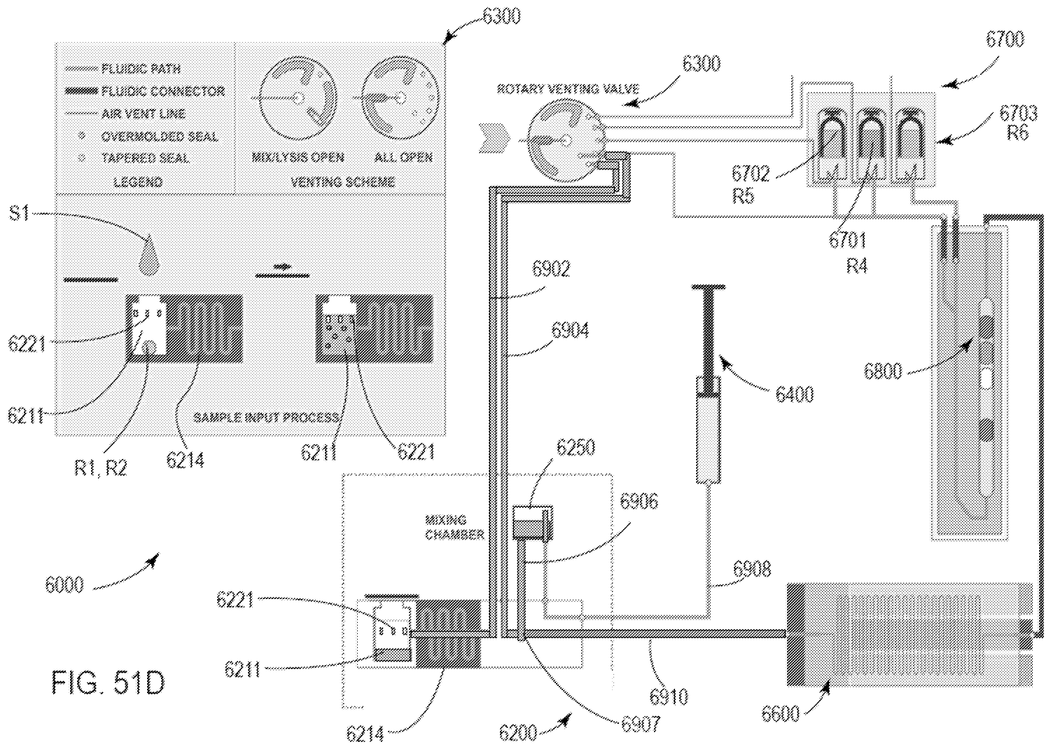

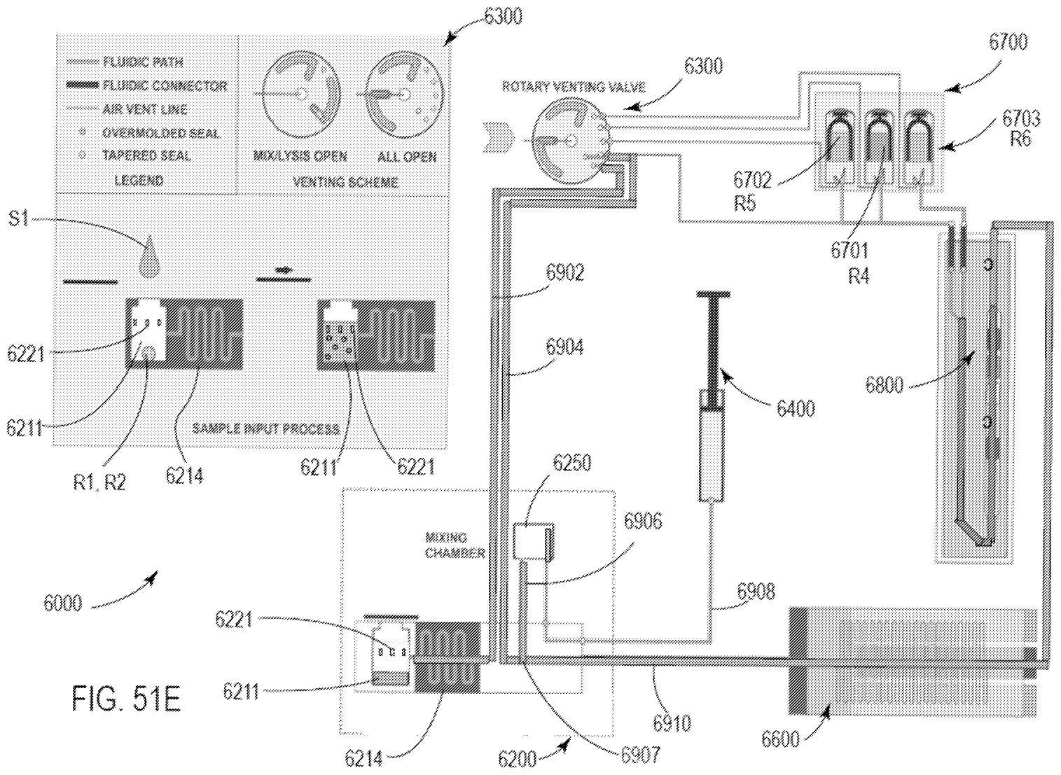

An example of the air flow paths and venting configuration is shown in FIGS. 53A-C, which show a mixing reservoir 7250 according to an embodiment. The mixing reservoir 7250 can be used in any of the devices described herein, including the device 6000 described above. The reservoir 7250 is defined by a mixing assembly housing 7251. The mixing assembly housing 7251 includes a first end portion 7253 and a second end portion 7255. The mixing assembly housing 7251 can be coupled within the housing of the device (e.g., the device 6000) such that when a base of the device housing is on a testing surface, the second end portion 7255 of the mixing housing 7251 is above the first end portion 7253 of the mixing housing. The first end portion 7253 of the mixing housing defines a mixing input opening 7252 and the second end portion 7255 of the mixing housing defining a mixing fluid drive internal opening 7269. Notably, the mixing reservoir of FIG. 29 includes a similar structure and is also applicable to the embodiments of device 6000.

Referring to FIGS. 53A-53C, the mixing assembly 7250 includes a bottom housing 7251, a top housing 7260, and operates without a vibration motor. The bottom housing 7251 defines a reservoir 7255 and contains the amplification reagents R3 therein. The bottom housing 7251 includes an inlet coupling 7252 and an fluid drive coupling 7253. The top housing 7260 encloses the mixing reservoir 7255. The inlet coupling 7252 and the fluid drive coupling 7253 can be constructed from any suitable material and can have any suitable size. For example, in some embodiments, the inlet coupling 7252 and the fluid drive coupling 7253 are constructed to limit the amount of vibration energy from the internal bubbling. For example, in some embodiments, the inlet coupling 7252 and the fluid drive coupling 7253 can be constructed from a resilient or elastomeric material.

In some embodiments, the mixing reservoir is in communication with an input reservoir such that the same fluidic drive (e.g., the fluidic drive 6400) can force air through both reservoirs. In some embodiments, the fluidic drive 6400 is in fluid communication with the mixing reservoir 6250 such that the fluidic drive 6400 forces air through the mixing reservoir 6250 in response to the molecular diagnostic test device 6000 being in a first configuration (e.g., first valve position). In some embodiments, the fluidic drive 6400 forces air through the input reservoir 6211 in response to the molecular diagnostic test device 6000 being in a second configuration (e.g., second valve position). In some embodiments, as illustrated in FIGS. 51A-E, the fluidic drive 6400 forces air through the mixing reservoir 6250 and the input reservoir 6211 in response to the molecular diagnostic test device 6000 being in a second configuration (e.g., second valve position). In some embodiments, the mixing reservoir 6250 is fluidically coupled to the vent via the mixing output opening (e.g., along flow channel 6906 to flow channel 6904) when the valve is in a first valve configuration. The mixing reservoir 6250 is fluidically coupled to the reaction module 6600 via the mixing output opening (e.g., along flow channel 6906 to flow channel 6910) when the valve is in a second valve configuration. As shown in FIGS. 51A-E, the fluid pump can be in serial fluid communication with the mixing reservoir and the input reservoir with the mixing reservoir positioned between the fluid pump and the input reservoir.

In some embodiments, the fluidic drive 6400 pulls air through the reservoirs. However, it is contemplated that with changes in venting and plumbing, the fluidic drive 6400 pushes air through the reservoirs. As shown in FIGS. 51A-E, air is pulled up through the mixing reservoir 6250. To do this air is pulled through flow channel 6908. Also shown in FIGS. 51A-51E is that air is pushed out though the input reservoir 6211. While, these configurations shown in the figures, it is understood that based on the disclosure herein, a person of skill in the art can vent and plumb the various reservoirs for the opposite effect.

Additionally or alternatively, the fluid transfer drive 6400 can then be operated in the opposite direction to pull the mixed biological sample out of the input reservoir 6211 and into the serpentine flow channel 6214. Additionally or alternatively, the fluid transfer drive 6400 can then be operated in the opposite direction to push the amplification solution out of the mixing reservoir 6250 and into the reaction module.

The fluid transfer drive 6400 is a piston pump including a piston that is an extended position within a syringe body to define the air reservoir prior to the biological sample being loaded into the reservoir, and the piston is configured to move in a first direction to force the air out of the syringe body and into the sample preparation module, the piston configured to move in a second direction opposite the first direction to pull the biological sample and reagent into the reaction module. The fluid transfer drive 6400 produces a force sufficient to convey a mixture of the biological sample and the reagent (e.g., the amplification solution) from the reservoir 6250 to the reaction module 6600 when the valve 6300 is in the second valve configuration.

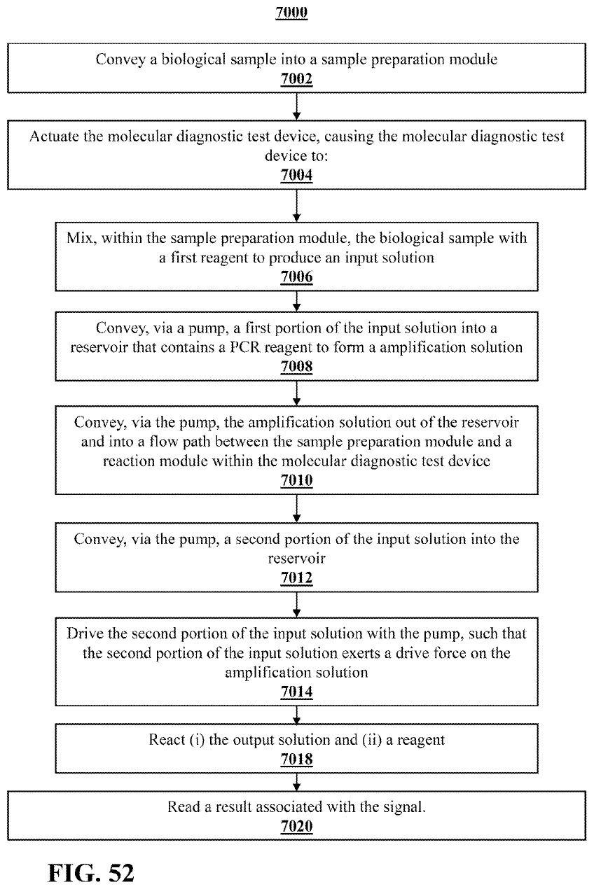

FIG. 54 is a flow chart illustrating a method 8000 for detecting a nucleic acid using a molecular diagnostic test device. The method includes conveying a biological sample into a sample preparation module within the molecular diagnostic test device via an input opening at 8002. The method also includes actuating the molecular diagnostic test device at 8004. The actuating also causes the molecular diagnostic test device to convey the biological sample into a reverse transcription flow path at 8006. The actuating also causes the molecular diagnostic test device to heat the biological sample via a heater of the sample preparation module within the reverse transcription flow path to produce an input solution at 8008. The actuating also causes the molecular diagnostic test device to convey the input solution into a reservoir that contains a reagent at 8010. The actuating also causes the molecular diagnostic test device to drive a fluid into the reservoir to mix the input solution and the reagent within the reservoir to produce an amplification solution at 8012. The actuating also causes the molecular diagnostic test device to heat the amplification solution within a reaction volume within the molecular diagnostic test device thereby producing an output solution containing a target amplicon at 8014. The actuating also causes the molecular diagnostic test device to react within the molecular diagnostic test device each of (i) the output solution and (ii) a reagent formulated to produce a signal that indicates a presence of the target amplicon within the output solution at 8016. The method also includes reading a result associated with the signal at 8018.

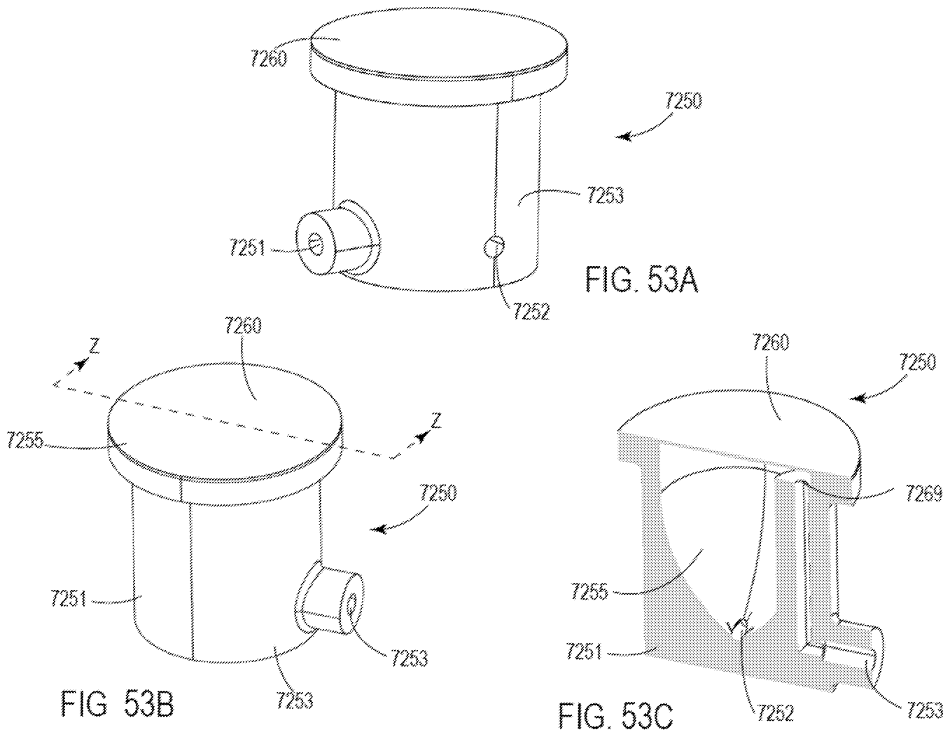

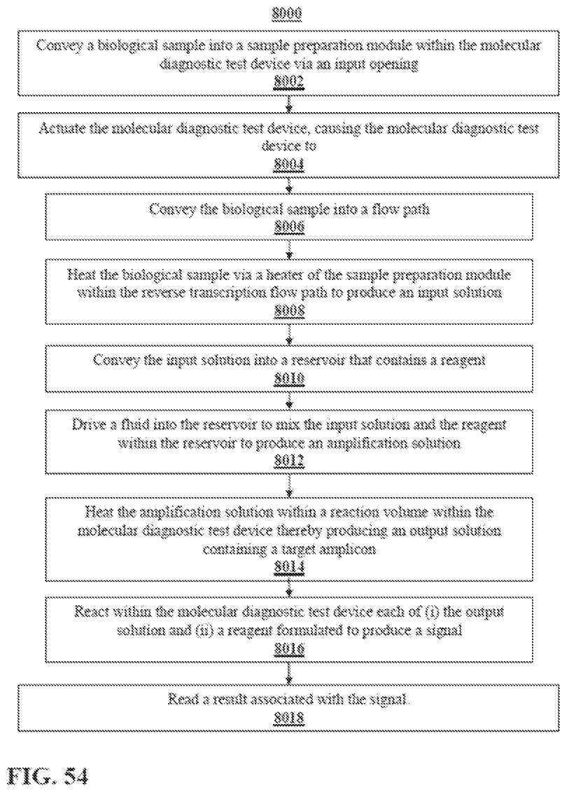

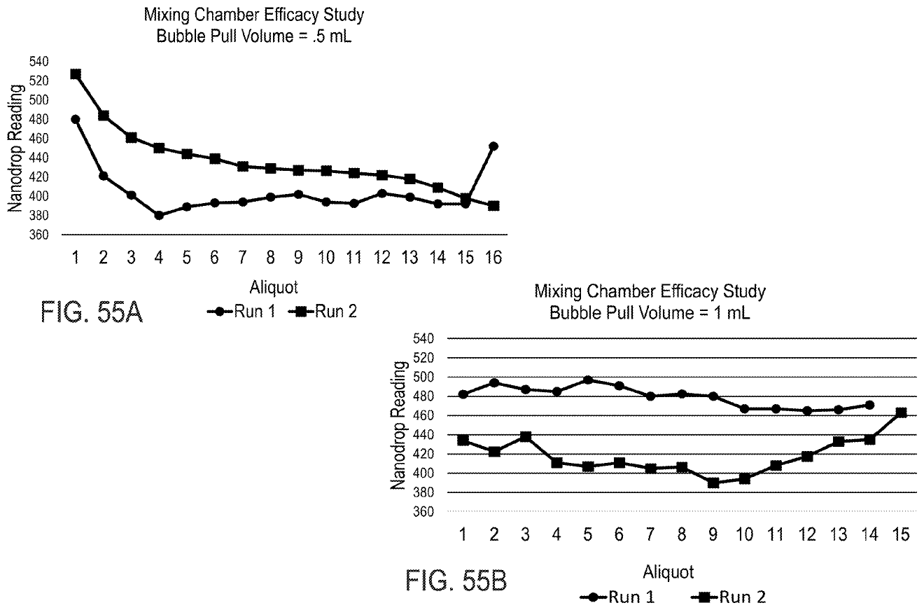

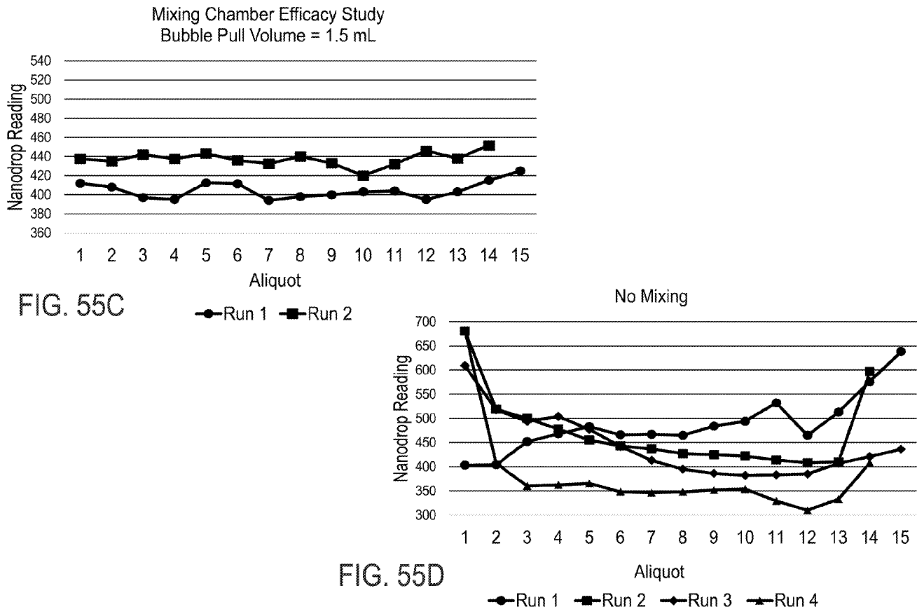

FIG. 55A shows the concentration of a solution mixed under a forced air in a mixing reservoir across multiple aliquots. More specifically, this is an example of limited mixing with only 0.5 mL of air from the fluid pump being pulled through the mixing reservoir. FIG. 55B shows the concentration of a solution mixed under a forced air in a mixing reservoir across multiple aliquots. More specifically, this is an example of mixing with 1.0 mL of air from the fluid pump being pulled through the mixing reservoir. FIG. 55C shows the concentration of a solution mixed under a forced air in a mixing reservoir across multiple aliquots. More specifically, this is an example of mixing with 1.5 mL of air from the fluid pump being pulled through the mixing reservoir. As can be seen from these example results consistent concentrations are improved based on additional mixing via air. The forced air mixing was performed using methods similar to those described herein. FIG. 55D shows the concentration of a solution when unmixed in a mixing reservoir across multiple aliquots for a controlled comparison to FIGS. 55A-C. In these examples the air is moved a about less than 15 μL/s In accordance with various embodiments, the air can be pushed through a mixing reservoir (e.g., reservoir 3250 or 7250) at between 10-20 μL/s. In some embodiments, the air can be pushed through the mixing reservoir at less than 15 μL/s. As shown in FIGS. 55A-C, the forced air mixing in the mixing reservoir results in the concentration of the reagent remaining closer to one another than what is illustrated in FIG. 55D with no mixing. FIGS. 55A-C provide more consistent samples than nonmixing. As shown in FIG. 55D, non-mixing tends to result in a in the concentration of the aliquots to be significantly different than one another. As can be seen in the figures, forced air mixing reduces this variation significantly.

While various embodiments have been described above, it should be understood that they have been presented by way of example only, and not limitation. Where methods and/or schematics described above indicate certain events and/or flow patterns occurring in certain order, the ordering of certain events and/or flow patterns may be modified. While the embodiments have been particularly shown and described, it will be understood that various changes in form and details may be made.

For example, any of the sample input modules, sample preparation modules, amplification modules, heater assemblies, and detection modules shown and described herein can be used in any suitable diagnostic device. Such devices can include, for example, a single-use device that can be used in a point-of-care setting and/or in a user's home. Similarly stated, in some embodiments, the device (and any of the other devices shown and described herein) can be configured for use in a decentralized test facility. Further, in some embodiments, any of the sample input modules, sample preparation modules, amplification modules, heater assemblies, and detection modules shown and described herein can be included within a CLIA-waived device and/or can facilitate the operation of a device in accordance with methods that are CLIA waived. Similarly stated, in some embodiments, the sample input modules, the sample preparation modules, the amplification modules, and the detection modules shown and described herein can facilitate operation of a device in a sufficiently simple manner that can produce results with sufficient accuracy to pose a limited likelihood of misuse and/or to pose a limited risk of harm if used improperly. In some embodiments, the sample input modules, the sample preparation modules, the amplification modules, and the detection modules shown and described herein can be used in any of the diagnostic devices shown and described in International Patent Publication No. WO2016/109691, entitled “Devices and Methods for Molecular Diagnostic Testing.”

Some embodiments described herein relate to a computer storage product with a non-transitory computer-readable medium (also can be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also can be referred to as code) may be those designed and constructed for the specific purpose or purposes. Examples of non-transitory computer-readable media include, but are not limited to: magnetic storage media such as hard disks, floppy disks, and magnetic tape: optical storage media such as Compact Disc/Digital Video Discs (CD/DVDs), Compact Disc-Read Only Memories (CD-ROMs), and holographic devices: magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices.

Examples of computer code include, but are not limited to, micro-code or microinstructions, machine instructions, such as produced by a compiler, code used to produce a web service, and files containing higher-level instructions that are executed by a computer using an interpreter. For example, embodiments may be implemented using imperative programming languages (e.g., C, Fortran, etc.), functional programming languages (Haskell, Erlang, etc.), logical programming languages (e.g., Prolog), object-oriented programming languages (e.g., Java, C++, etc.) or other suitable programming languages and/or development tools. Additional examples of computer code include, but are not limited to, control signals, encrypted code, and compressed code.

The processor included within a control module (and any of the processors and/or controllers described herein) can be any processor configured to, for example, write data into and read data from the memory of the controller, and execute the instructions and/or methods stored within the memory. Furthermore, the processor can be configured to control operation of the other modules within the controller (e.g., the temperature feedback module and the flow module). Specifically, the processor can receive a signal including temperature data, current measurements or the like and determine an amount of power and/or current to be supplied to each heater assembly, the desired timing and sequence of the piston pulses and the like. For example, in some embodiments, the controller can be an 8-bit PIC microcontroller, an ARM Cortex M0+ or other suitable controllers, which will control the power delivered to various heating assemblies and components within the amplification module. This microcontroller can also contain code for and/or be configured to minimize the instantaneous power requirements on the power source.

In other embodiments, any of the processors described herein can be, for example, an application-specific integrated circuit (ASIC) or a combination of ASICs, which are designed to perform one or more specific functions. In yet other embodiments, the microprocessor can be an analog or digital circuit, or a combination of multiple circuits.

Any of the memory devices described herein can be any suitable device such as, for example, a read only memory (ROM) component, a random access memory (RAM) component, electronically programmable read only memory (EPROM), erasable electronically programmable read only memory (EEPROM), registers, cache memory, and/or flash memory. Any of the modules (the pressure feedback module and the position feedback module) can be implemented by the processor and/or stored within the memory.

Although various embodiments have been described as having particular features and/or combinations of components, other embodiments are possible having a combination of any features and/or components from any of embodiments as discussed above.

1. A method of detecting a nucleic acid using a molecular diagnostic test device, comprising:
conveying a biological sample into a sample preparation module within the molecular diagnostic test device via an input opening;
actuating the molecular diagnostic test device, causing the molecular diagnostic test device to:
convey the biological sample into a reverse transcription flow path;
heat the biological sample via a heater of the sample preparation module within the reverse transcription flow path to produce an input solution containing cDNA;
convey the input solution containing cDNA into a reservoir that contains a reagent;
drive a fluid into the reservoir to mix the input solution and the reagent within the reservoir to produce an amplification solution;
heat the amplification solution within a reaction volume within the molecular diagnostic test device to amplify the cDNA within the amplification solution thereby producing an output solution containing a target amplicon; and
react within the molecular diagnostic test device each of (i) the output solution and (ii) a reagent formulated to produce a signal that indicates a presence of the target amplicon within the output solution; and
reading a result associated with the signal.

2. The method of claim 1, wherein:
the reagent is a first reagent, the reservoir is a mixing reservoir and the biological sample is received into an input reservoir that contains a second reagent; and
the actuating the molecular diagnostic test device further causes the molecular diagnostic test device to:
mix the second reagent with the biological sample; and
convey the biological sample out of the input reservoir and into the reverse transcription flow path.

3. The method of claim 2, wherein the fluid is air, and the air is driven into the mixing reservoir by a fluid pump thereby using air bubbles within the biological sample to agitate the biological sample and the reagent.

4. The method of claim 3, wherein the actuating the molecular diagnostic test device further causes the molecular diagnostic test device to:
drive, via the fluid pump, the fluid into the input reservoir to mix the biological sample and the reagent within the reservoir.

5. The method of claim 4, wherein the biological sample is loaded in a first direction within a flow path into the input reservoir and the fluid is driven into the input reservoir from a second direction along the flow path.

6. The method of claim 5, wherein the fluid pump is in fluid communication with the mixing reservoir and the input reservoir such that the fluid pump forces the air through the mixing reservoir in response to the molecular diagnostic test device being in a first configuration and the fluid pump forces air through the input reservoir in response to the molecular diagnostic test device being in a second configuration.

7. The method of claim 6, wherein the fluid pump is in serial fluid communication with the mixing reservoir and the input reservoir with the mixing reservoir positioned between the fluid pump and the input reservoir.

8. The method of claim 6, wherein:
the pump includes a piston and a syringe body, the pump contains a volume of the air prior to conveying the biological sample;
the piston being moved in a first piston direction in response to actuation of the molecular diagnostic test device to drive the air into the input reservoir along the reverse transcription flow path causing bubbles in the input reservoir; and
the piston being moved in a second piston direction to convey the biological sample into the reverse transcription flow path.

9. The method of claim 8, wherein the molecular diagnostic test device further comprises:
a valve in fluid communication with the mixing reservoir, the valve being configured to vent an inlet into the mixing reservoir while the piston is moved causing air to be pulled through the vent into the reservoir.

10. The method of claim 9, wherein the molecular diagnostic test device includes a control module, the control module including a switch and a processor, the processor configured to control transmission of power to the pump of the molecular diagnostic test device for driving air to at least one of the input reservoir or the mixing reservoir.

11. A molecular diagnostic test device, comprising:
a housing;
a sample preparation module within the housing, the sample preparation module defining a sample input opening through which a biological sample can be loaded and a reservoir containing a reagent;
a fluid source within the housing, the fluid source being in fluid communication with the reservoir such that during operation of the molecular diagnostic test device, fluid from the fluid source is injected into the reservoir to mix the biological sample and the reagent within the reservoir to form a solution containing a target molecule for amplification; and
a reaction module disposed within the housing, the reaction module including a reaction volume, the reaction module configured to receive the solution containing the target molecule and amplify the target molecule to produce an output containing a target amplicon.

12. The molecular diagnostic test device of claim 11, wherein the fluid source includes a fluid pump and an air reservoir within the fluid pump in fluid communication with the reservoir such that air from the air reservoir is pumpable into the reservoir.

13. The molecular diagnostic test device of claim 12, wherein the air forms bubbles in the reservoir agitating the biological sample and the reagent.

14. The molecular diagnostic test device of claim 12, wherein the fluid pump is a piston pump including a piston that is an extended position within a syringe body to define the air reservoir prior to the biological sample being loaded into the reservoir, and the piston is configured to move in a first direction to force the air out of the syringe body and into the sample preparation module, the piston configured to move in a second direction opposite the first direction to pull the biological sample and reagent into the reaction module.

15. The molecular diagnostic test device of claim 14, wherein:
the reagent is a second reagent;
the reservoir is a mixing reservoir that is in communication with an input reservoir; and
the input reservoir contains a first reagent.

16. The molecular diagnostic test device of claim 15, wherein the fluid pump is in fluid communication with the mixing reservoir such that the fluid pump forces air through the mixing reservoir in response to the molecular diagnostic test device being in a first configuration and the fluid pump forces air through the input reservoir in response to the molecular diagnostic test device being in a second configuration.

17. The molecular diagnostic test device of claim 16, wherein the fluid pump is in serial fluid communication with the mixing reservoir and the input reservoir with the mixing reservoir positioned between the fluid pump and the input reservoir.

18. The molecular diagnostic test device of claim 16, wherein the reservoir is defined by a mixing assembly housing, the mixing assembly housing having a first end portion and a second end portion, the mixing assembly housing coupled within the housing such that when a base of the housing is on a testing surface, the second end portion of the mixing housing is above the first end portion of the mixing housing, the first end portion of the mixing housing defining a mixing input opening and the second end portion of the mixing housing defining a fluid drive opening, the molecular diagnostic test device further comprising:
a valve configured to selectively fluidically couple the air reservoir to the mixing reservoir via the mixing input opening, the valve configured to selectively fluidically couple the mixing reservoir to a vent via the mixing output opening.

19. The molecular diagnostic test device of claim 18, wherein:
the mixing reservoir is fluidically coupled to the vent via the mixing output opening when the valve is in a first valve configuration;
the mixing reservoir is fluidically coupled to the reaction volume via the mixing output opening when the valve is in a second valve configuration; and
the fluid pump is configured to produce a force to convey a mixture of the biological sample and the reagent from the reservoir to the reaction module when the valve is in the second valve configuration.

20-33. (canceled)

34. An apparatus, comprising:
a housing having an opening;
a sample preparation module within the housing, the sample preparation module defining a sample input opening aligned with the housing opening and an input reservoir, the sample preparation module including a retention screen positioned to separate the input reservoir into a first portion and a second portion, the sample input opening being within the first portion, the second portion containing a solid reagent, the sample preparation module configured to receive a biological sample and to mix the biological sample with the solid reagent to form an input solution containing a target molecule; and
a reaction module disposed within the housing, the reaction module configured to receive the input solution from the second portion of the input reservoir and amplify the target molecule to produce an output containing a target amplicon.

35. The apparatus of claim 34, wherein the retention screen defines a plurality of apertures that are sized to allow the biological sample to flow through the retention screen from the first portion of the input reservoir to the second portion of the input reservoir, while also maintaining the solid reagent within the second portion of the input reservoir.

36. (canceled)

37. The apparatus of claim 34, wherein:
the input reservoir has a height; and
the retention screen is positioned within the input reservoir at a screen distance below the sample input opening, a ratio of the screen distance to the height being between 0.3 and 0.6.

38-79. (canceled)
